(19) United States
(12) Patent Application Publication
(10) Pub. No.: US 2023/0087321 A1
(43) Pub. Date: Mar. 23, 2023

(54) ODORLESS CANNABIS PLANT

(71) Applicant: BETTERSEEDS LTD., Givat Chen (IL)

(72) Inventors: Ido MARGALIT, Gan Yavne (IL); Tal SHERMAN, Rehovot (IL)

(73) Assignee: BETTERSEEDS LTD., Givat Chen (IL)

(21) Appl. No.: 17/905,346

(22) PCT Filed: Mar. 2, 2021
(86) PCT No.: PCT/IL2021/050234

§ 371 Date: Aug. 31, 2022

Related U.S. Application Data
(60) Provisional application No. 62/984,346, filed on Mar. 3, 2020.

(51) Int. Cl. A01H 6/28; A01H 5/10; C12N 9/22; C12N 15/82

(52) U.S. Cl. CPC A01H 6/28; A01H 5/10; C12N 9/22; C12N 15/8243

(57) ABSTRACT

Provided is a modified Cannabis plant exhibiting reduced volatile organic compounds (VOCs) emission. The modified Cannabis plant includes at least one targeted gene modification conferring reduced expression or silencing of at least one gene involved in a terpene biosynthesis pathway. Further provided are methods and uses concerning the aforementioned modified Cannabis plant.

### FIELD OF THE INVENTION

The present disclosure relates to a method of silencing terpene synthesis genes. The present disclosure further concerns the generation of odor free Cannabis plants using genome-editing techniques.

### BACKGROUND OF THE INVENTION

The Cannabis market is enjoying an unprecedented spike in activity following the wide spread legalization trend across the world. The American market alone is estimated to reach an exceptional growth rate of 30% per annum. This has led to an increase in demand not only for Cannabis products in general but in particular for products with specific traits, for medicinal or recreational use.

It is well known that the Cannabis plant emits a very strong odor, mainly due to the release of chemical compounds into the air known as volatile organic compounds (VOCs). A study by Rice et al. identified over 200 different VOCs from packaged Cannabis samples. Odor emissions are a nuisance and complaints from nearby residents are harming the industry. The strong odors produced by growing cannabis can be difficult to manage. Described as pungent, skunky, floral, fruity or even “sewer-like,” these odors are labeled a nuisance. Some odors from Cannabis farms have been detected more than a mile from their source. Moreover, complaints of Cannabis odors have increased in some areas by as much as 87% since growing marijuana became legal. Thus reducing Cannabis odors is a growing concern.

Current practices recommend the use of appropriate ventilation and filtration systems at Cannabis production/cultivation facilities to mitigate the release of substances that may result in odors. Systems to report and track odors could help inform on timing and extent of the occurrence of odor to assist local authorities to remedy potential problems. No studies on health effects associated with exposure to Cannabis odors were identified in the literature. An important consideration when sampling for odorous compounds is the possibility that compounds emitted at higher concentrations may not necessarily be responsible for the overall characteristic of the odor. In addition, the overall odor of Cannabis can be time dependent as chemical volatilization occurs at different rates for different compounds. While both fresh and dry Cannabis can be associated with odors it is possible that the VOCs responsible for the aroma profiles may be different due to different rates of chemical volatilization. As a result, it is difficult to identify one or a selected number of chemicals to measure from a facility to potentially monitor odor on a continuous basis (Public Health Ontario, 2018).

In lieu of the above, there is still a long felt need to provide novel methods of effectively and consistently eliminating volatile compounds such as terpenes in a Cannabis plant.

### SUMMARY OF THE INVENTION

It is therefore one object of the present invention to disclose a modified Cannabis plant exhibiting reduced volatile organic compounds (VOCs) emission, wherein the modified plant comprises at least one targeted gene modification conferring reduced expression or silencing of at least one gene involved in a terpene biosynthesis pathway.

It is a further object of the present invention to disclose the modified Cannabis plant as defined above, wherein the at least one targeted gene modification confers reduced expression or silencing of at least one gene involved in a terpene biosynthesis pathway as compared to a Cannabis plant lacking the targeted gene modification.

It is a further object of the present invention to disclose the modified Cannabis plant as defined in any of the above, wherein the terpene biosynthesis pathway is selected from methylerythritol phosphate (MEP) pathway, mevalonic acid or mevalonate (MEV) pathway, isoprenoid biosynthetic pathway, formation of GPP, FPP and GGPP pathways, formation of squalene pathway, formation of Mono-, Sesqui-und Di-Terpenes pathways, formation of triterpenes from squalene pathway and any combination thereof.

It is a further object of the present invention to disclose the modified Cannabis plant as defined in any of the above, wherein one gene involved in a terpene biosynthesis pathway is selected from CsTPS1PK, CsTPS4PK, CsTPS5PK, CsTPS6PK, CsTPS7PK, CsTPS8PK, CsTPS9PK, CsTPS10PK, CsTPS11PK, CsTPS12PK, CsTPS13PK, CsTPS14PK, CsTPS15PK, CsTPS16PK, CsTPS17PK, CsTPS18PK, CsTPS19PK, CsTPS20PK, CsTPS21PK, CsTPS22PK, CsTPS23PK, CsTPS24PK, CsTPS25PK, CsTPS26PK, CsTPS27PK, CsTPS30PK, CsTPS31PK, CsTPS32PK, CsTPS33PK, CsTPS34PK, CsTPS35PK, CsTPS12PK, CsTPS13PK, CsTPS1FN, CsTPS2FN, CsTPS3FN, CsTPS4FN, CsTPS5FN, CsTPS6FN, CsTPS7FN, CsTPS8FN, CsTPS9FN, CsTPS1 FN, CsDXS1, CsDXS2, CsDXR, CsMCT, CsCMK, CsHDS, CsHDR, CsHMGS, CsHMGR1, CsHMGR2, CsMK, CsPMK, CsMPDC, CsIDI, CsFPPS1, CsFPPS2, CsGPPS1, CsGPPS2 and any combination thereof.

It is a further object of the present invention to disclose the modified Cannabis plant as defined in any of the above, wherein the gene involved in a terpene biosynthesis pathway is selected from (a) a gene encoding CsFPPS1 characterized by a sequence selected from SEQ ID NO: 1-3 or a functional variant thereof, (b) a gene encoding CsFPPS2 characterized by a sequence selected from SEQ ID NO: 4-6 or a functional variant thereof, (c) a gene encoding CsGPPS1 characterized by a sequence selected from SEQ ID NO: 7-9 or a functional variant thereof, (d) a gene encoding CsGPPS2 characterized by a sequence selected from SEQ ID NO: 10-12 or a functional variant thereof, and (e) any combination thereof.

It is a further object of the present invention to disclose the modified Cannabis plant as defined in any of the above, wherein the functional variant has at least 75% sequence identity to the gene sequence.

It is a further object of the present invention to disclose the modified Cannabis plant as defined in any of the above, wherein the gene modification is introduced using mutagenesis, small interfering RNA (siRNA), microRNA (miRNA), artificial miRNA (amiRNA), DNA introgression, endonucleases or any combination thereof.

It is a further object of the present invention to disclose the modified Cannabis plant as defined in any of the above, wherein the gene modification is introduced using CRISPR (Clustered Regularly Interspaced Short Palindromic Repeats) and CRISPR-associated (Cas) gene (CRISPR/Cas) system, Transcription activator-like effector nuclease (TALEN), Zinc Finger Nuclease (ZFN), meganuclease or any combination thereof.

It is a further object of the present invention to disclose the modified Cannabis plant as defined in any of the above, wherein the CRISPR/Cas system is delivered to the Cannabis plant or cell thereof by a plant virus vector.

It is a further object of the present invention to disclose the modified Cannabis plant as defined in any of the above, wherein the Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi) and any combination thereof.

It is a further object of the present invention to disclose the modified Cannabis plant as defined in any of the above, wherein the targeted gene modification is introduced using (i) at least one RNA-guided endonuclease, or a nucleic acid encoding at least one RNA-guided endonuclease, and (ii) at least one guide RNA (gRNA) or DNA encoding at least one gRNA which directs the endonuclease to a corresponding target sequence within the gene involved in terpene biosynthesis pathway.

It is a further object of the present invention to disclose the modified Cannabis plant as defined in any of the above, wherein the targeted gene modification is performed by introducing into a Cannabis plant or a cell thereof a nucleic acid composition comprising: a) a first nucleotide sequence encoding the targeted gRNA molecule and b) a second nucleotide sequence encoding the Cas molecule, or a Cas protein.

It is a further object of the present invention to disclose the modified Cannabis plant as defined in any of the above, wherein the gRNA comprises a sequence selected from SEQ ID NO:13-646 and any combination thereof.

It is a further object of the present invention to disclose the modified Cannabis plant as defined in any of the above, wherein the gRNA targeted for CsFPPS1, CsFPPS2, CsGPPS1 and/or CsGPPS2 comprises a nucleic acid sequence as set forth in SEQ ID NO: 13-237, SEQ ID NO: 238-390, SEQ ID NO: 391-530 and SEQ ID NO: 531-646, respectively.

It is a further object of the present invention to disclose the modified Cannabis plant as defined in any of the above, wherein the targeted gene modification is introduced into the Cannabis plant or a cell thereof using an expression cassette or construct comprising (a) Cas DNA and gRNA sequence selected from the group consisting of SEQ ID NO:13-646 and any combination thereof, or (b) a ribonucleoprotein (RNP) complex comprising Cas protein and gRNA sequence selected from the group consisting of SEQ ID NO:13-646 and any combination thereof.

It is a further object of the present invention to disclose the modified Cannabis plant as defined in any of the above, wherein the gene modification is introduced using an expression cassette comprising a) a nucleotide sequence encoding one or more gRNA molecules comprising a DNA sequence which is complementary with a target domain sequence within a gene selected from CsFPPS1, CsFPPS2, CsGPPS1 and CsGPPS2, and b) a nucleotide sequence encoding a Cas molecule, or a Cas protein.

It is a further object of the present invention to disclose the modified Cannabis plant as defined in any of the above, wherein, the target domain sequence within the Cannabis genome is selected from the group comprising of 1) a nucleic acid sequence encoding the polypeptide of CsFPPS1, the nucleic acid having a sequence as set forth in SEQ ID NO: 1 (2) a nucleic acid sequence encoding the polypeptide of CsFPPS2, the nucleic acid having a sequence as set forth in SEQ ID NO: 4 (3) a nucleic acid sequence encoding the polypeptide of CsGPPS1, the nucleic acid having a sequence as set forth in SEQ ID NO: 7 (4) a nucleic acid sequence encoding the polypeptide of CsGPPS2, the nucleic acid having a sequence as set forth in SEQ ID NO: 10 (5) a nucleic acid sequence having at least 80% sequence identity to at least 200 contiguous nucleotides of the nucleic acid sequence of CsFPPS1, (6) a nucleic acid sequence having at least 80% sequence identity to at least 200 contiguous nucleotides of the nucleic acid sequence of CsFPPS2, (7) a nucleic acid sequence having at least 80% sequence identity to at least 200 contiguous nucleotides of the nucleic acid sequence of CsGPPS1, (8) a nucleic acid sequence having at least 80% sequence identity to at least 200 contiguous nucleotides of the nucleic acid sequence of CsGPPS2.

It is a further object of the present invention to disclose the modified Cannabis plant as defined in any of the above, wherein the gRNA sequence comprises a 3′ Protospacer Adjacent Motif (PAM) selected from the group consisting of NGG (SpCas), NNNNGATT (NmeCas9), NNAGAAW, (StCas9), NAAAAC (TdCas9), NNGRRT (SaCas9) and TBN (Cas-phi).

It is a further object of the present invention to disclose the modified Cannabis plant as defined in any of the above, wherein the targeted gene modification is a CRISPR/Cas9-induced heritable mutated allele of at least one of CsFPPS1, CsFPPS2, CsGPPS1 and/or CsGPPS2 encoding gene.

It is a further object of the present invention to disclose the modified Cannabis plant as defined in any of the above, wherein the targeted gene modification is a missense mutation, nonsense mutation, insertion, deletion, indel, substitution or duplication.

It is a further object of the present invention to disclose the modified Cannabis plant as defined in any of the above, wherein the insertion, deletion or indel produces a gene comprising a frameshift.

It is a further object of the present invention to disclose the modified Cannabis plant as defined in any of the above, wherein the targeted gene modification is in the coding region of the gene, in the regulatory region of the gene, in a gene downstream or upstream of the corresponding gene in the terpene biosynthesis pathway and/or an epigenetic factor.

It is a further object of the present invention to disclose the modified Cannabis plant as defined in any of the above, wherein the targeted gene modification is a silencing mutation, a knockdown mutation, a knockout mutation, a loss of function mutation or any combination thereof.

It is a further object of the present invention to disclose the modified Cannabis plant as defined in any of the above, wherein the Cannabis plant is selected from the group of species that includes, but is not limited to, Cannabis sativa (C. sativa), C. indica, C. ruderalis and any hybrid or cultivated variety of the genus Cannabis.

It is a further object of the present invention to disclose the modified Cannabis plant as defined in any of the above, wherein the expression of the at least one gene involved in a terpene biosynthesis pathway is eliminated.

It is a further object of the present invention to disclose the modified Cannabis plant as defined in any of the above, wherein the modified plant has reduced odor resulting from volatile compounds emission or is odor free or odorless Cannabis plant.

It is a further object of the present invention to disclose the modified Cannabis plant as defined in any of the above, wherein the VOCs are selected from essential oils, secondary metabolites, terpenoids, terpenes, oxygenated and any combination thereof.

It is a further object of the present invention to disclose the modified Cannabis plant as defined in any of the above, wherein VOCs comprise at least one of hemiterpenes, monoterpenes, sesquiterpenes, diterpenes, sesterterpenes, triterpenes, tetraterpenes and polyterpenes.

It is a further object of the present invention to disclose the modified Cannabis plant as defined in any of the above, wherein the VOCs are selected from pinene, alpha-pinene, beta-pinene, cis-pinane, trans-pinane, cis-pinanol, trans-pinanol, limonene; linalool; myrcene; eucalyptol; a-phellandrene; b-phellandrene; a-ocimene; b-ocimene, cis-ocimene, ocimene, delta-3-carene; fenchol; sabinene, bomeol, isobomeol, camphene, camphor, phellandrene, a-phellandrene, a-terpinene, geraniol, linalool, nerol, menthol, terpinolene, a-terpinolene, b-terpinolene, g-terpinolene, delta-terpinolene, a-terpineol, trans-2-pinanol, caryophyllene, caryophyllene oxide, humulene, a-humulene, a-bisabolene; b-bisabolene; santalol; selinene; nerolidol, bisabolol; a-cedrene, b-cedrene, b-eudesmol, eudesm-7(11)-en-4-ol, selina-3,7(11)-diene, guaiol, valencene, a-guaiene, beta-guaiene, delta-guaiene, guaiene, famesene, a-famesene, b-famesene, elemene, a-elemene, b-elemene, gamma-elemene, delta-elemene, germacrene, germacrene A, germacrene B, germacrene C, germacrene D, germacrene E, oridonin, phytol, isophytol, ursolic acid, oleanolic acid, and/or 1.5 ene compounds, including guaia-1(10),11-diene, and 1.5 ene. Guaia-1(10), 11-diene.isoprene, α-pinene, β-pinene, d-limonene, β-phellandrene, α-terpinene, α-thujene, γ-terpinene, β-myrcene, (E)-β-ocimene, (−)-limonene, (+)-α-pinene, β-caryophyllene, and α-humulene and any combination thereof.

It is a further object of the present invention to disclose the modified Cannabis plant as defined in any of the above, wherein the Cannabis plant does not comprise a transgene within its genome.

It is a further object of the present invention to disclose the modified Cannabis plant as defined in any of the above, wherein the VOCs in the modified Cannabis plant is measured using gas chromatography-mass spectrometry (GCMS) terpene profiling and quantitation techniques or by any other method for quantifying VOCs.

It is a further object of the present invention to disclose a progeny plant, plant part, plant cell or plant seed of a modified plant as defined in any of the above.

It is a further object of the present invention to disclose a tissue culture of regenerable cells, protoplasts or callus obtained from the modified Cannabis plant as defined in any of the above.

It is a further object of the present invention to disclose the modified Cannabis plant as defined in any of the above, wherein the plant genotype is obtainable by deposit under accession number with NCIMB Aberdeen AB21 9YA, Scotland, UK.

It is a further object of the present invention to disclose the modified Cannabis plant as defined in any of the above, wherein the gene modification of CsFPPS1, CsFPPS2, CsGPPS1 and/or CsGPPS2 genes does not involve insertion of exogenous genetic material and produces a non-naturally occurring Cannabis plant or cell thereof.

It is a further object of the present invention to disclose a medical Cannabis product comprising the modified Cannabis plant as defined in any of the above or a part or extract thereof.

It is a further object of the present invention to disclose a method for producing a modified Cannabis plant as defined in any of the above, the method comprises introducing using targeted genome modification, at least one genomic modification conferring reduced expression or silencing of at least one gene involved in a terpene biosynthesis pathway.

It is a further object of the present invention to disclose a method for producing a modified Cannabis plant exhibiting reduced volatile organic compounds (VOCs) emission, the method comprises introducing into Cannabis plant cell, using targeted genome modification, at least one genomic modification conferring reduced expression or silencing of at least one gene involved in a terpene biosynthesis pathway.

It is a further object of the present invention to disclose the method as defined in any of the above, comprising steps of introducing using genome editing a loss of function mutation in at least one gene involved in a terpene biosynthesis pathway.

It is a further object of the present invention to disclose the method as defined in any of the above, wherein the method comprises steps of: (a) identifying at least one Cannabis gene involved in a terpene biosynthesis pathway; (b) designing and/or synthetizing at least one guide RNA (gRNA) comprising a nucleotide sequence corresponding or complementary to a target sequence is the at least one identified Cannabis gene involved in a terpene biosynthesis pathway; (c) transforming a Cannabis plant cells with endonuclease or nucleic acid encoding endonuclease, together with the at least one gRNA or a DNA encoding the gRNA; (d) optionally, culturing the transformed Cannabis cells; (e) selecting Cannabis plant or plant cells thereof carrying induced targeted loss of function mutation in the at least one gene involved in a terpene biosynthesis pathway; and (f) optionally, regenerating a modified Cannabis plant from the transformed plant cell, plant cell nucleus, or plant tissue.

It is a further object of the present invention to disclose the method as defined in any of the above, further comprises steps of screening the genome of the transformed Cannabis plant or plant cells thereof for induced targeted loss of function mutation in the at least one gene involved in a terpene biosynthesis pathway.

It is a further object of the present invention to disclose the method as defined in any of the above, further comprises steps of screening the regenerated plants for a Cannabis plant with reduced volatile organic compounds (VOCs) emission.

It is a further object of the present invention to disclose the method as defined in any of the above, comprising steps of introducing into a Cannabis plant or plant cells thereof a construct or expression cassette comprising (a) Cas nucleotide sequence operably linked to the at least one gRNA, or (b) a ribonucleoprotein (RNP) complex comprising Cas protein and the at least one gRNA.

It is a further object of the present invention to disclose the method as defined in any of the above, wherein the step of screening the genome of the transformed plant cells for induced targeted loss of function mutation further comprises steps of obtaining a nucleic acid sample of the transformed plant and performing a nucleic acid amplification and optionally restriction enzyme digestion to detect a mutation in the at least one gene involved in a terpene biosynthesis pathway.

It is a further object of the present invention to disclose the method as defined in any of the above, wherein the terpene biosynthesis pathway is selected from methylerythritol phosphate (MEP) pathway, mevalonic acid or mevalonate (MEV) pathway, isoprenoid biosynthetic pathway, formation of GPP, FPP and GGPP pathways, formation of squalene pathway, formation of Mono, Sesqui-und Di-Terpenes pathways, formation of triterpenes from squalene pathway and any combination thereof.

It is a further object of the present invention to disclose the method as defined in any of the above, wherein one gene involved in a terpene biosynthesis pathway is selected from CsTPS1PK, CsTPS4PK, CsTPS5PK, CsTPS6PK, CsTPS7PK, CsTPS8PK, CsTPS9PK, CsTPS10PK, CsTPS11PK, CsTPS12PK, CsTPS13PK, CsTPS14PK, CsTPS15PK, CsTPS16PK, CsTPS17PK, CsTPS18PK, CsTPS19PK, CsTPS20PK, CsTPS21PK, CsTPS22PK, CsTPS23PK, CsTPS24PK, CsTPS25PK, CsTPS26PK, CsTPS27PK, CsTPS30PK, CsTPS31PK, CsTPS32PK, CsTPS33PK, CsTPS34PK, CsTPS35PK, CsTPS12PK, CsTPS13PK, CsTPS1FN, CsTPS2FN, CsTPS3FN, CsTPS4FN, CsTPS5FN, CsTPS6FN, CsTPS7FN, CsTPS8FN, CsTPS9FN, CsTPS11FN, CsDXS1, CsDXS2, CsDXR, CsMCT, CsCMK, CsHDS, CsHDR, CsHMGS, CsHMGR1, CsHMGR2, CsMK, CsPMK, CsMPDC, CsIDI, CsFPPS1, CsFPPS2, CsGPPS1, CsGPPS2 and any combination thereof.

It is a further object of the present invention to disclose the method as defined in any of the above, wherein the gene involved in a terpene biosynthesis pathway is selected from (a) a gene encoding CsFPPS1 characterized by a sequence selected from SEQ ID NO: 1-3 or a functional variant thereof, (b) a gene encoding CsFPPS2 characterized by a sequence selected from SEQ ID NO: 4-6 or a functional variant thereof, (c) a gene encoding CsGPPS1 characterized by a sequence selected from SEQ ID NO: 7-9 or a functional variant thereof, (d) a gene encoding CsGPPS2 characterized by a sequence selected from SEQ ID NO: 10-12 or a functional variant thereof, and (e) any combination thereof.

It is a further object of the present invention to disclose the method as defined in any of the above, wherein the functional variant has at least 75% sequence identity to the gene sequence.

It is a further object of the present invention to disclose the method as defined in any of the above, wherein the gRNAs targeted for CsFPPS1, CsFPPS2, CsGPPS1 and CsGPPS2 comprising a SEQ ID NO: 13-237, SEQ ID NO: 238-390, SEQ ID NO: 391-530 and SEQ ID NO: 531-646, respectively.

It is a further object of the present invention to disclose the method as defined in any of the above, wherein the transformation into Cannabis plant or plant cells thereof is carried out using Agrobacterium or biolistics to deliver an expression cassette comprising a) a selection marker, b) a nucleotide sequence encoding one or more gRNA molecules comprising a DNA sequence complementary to a target domain sequence within a gene selected from CsFPPS1, CsFPPS2, CsGPPS1 and CsGPPS2, c) a nucleotide sequence encoding a Cas molecule.

It is a further object of the present invention to disclose the method as defined in any of the above, further comprises introduction into a Cannabis plant cell a construct or expression cassette comprising (a) Cas DNA and gRNA sequence selected from the group consisting of SEQ ID NO:13-646 and any combination thereof, or (b) a ribonucleoprotein (RNP) complex comprising Cas protein and gRNA sequence selected from the group consisting of SEQ ID NO:13-646 and any combination thereof.

It is a further object of the present invention to disclose the method as defined in any of the above, wherein the RNA-guided endonuclease is derived from a clustered regularly interspersed short palindromic repeats (CRISPR)/CRISPR-associated (Cas) system.

It is a further object of the present invention to disclose the method as defined in any of the above, wherein the Cas encoding gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi) and any combination thereof.

It is a further object of the present invention to disclose the method as defined in any of the above, wherein editing of CsFPPS1, CsFPPS2, CsGPPS1 and/or CsGPPS2 genes does not involve insertion of exogenous genetic material and produces a non-naturally occurring Cannabis plant or cell thereof.

It is a further object of the present invention to disclose the method as defined in any of the above, comprises silencing or eliminating Cannabis terpene synthesis gene expression comprising steps of: (a) identifying at least one gene locus within a DNA sequence in a Cannabis plant or a cell thereof for CsFPPS1, CsFPPS2, CsGPPS1 and/or CsGPPS2 having a genomic sequence as set for in SEQ ID NO:1, 4, 7 and 10, respectively; (b) identifying at least one custom endonuclease recognition sequence within the at least one locus of CsFPPS1, CsFPPS2, CsGPPS1 and/or CsGPPS2 genes; (c) introducing into the Cannabis plant or a cell thereof at least a first custom gRNA directed endonuclease, wherein the Cannabis plant or a cell thereof comprises the recognition sequence for the custom gRNA directed endonuclease in or proximal to the loci of any one of SEQ ID NO:13-646, and the custom endonuclease is expressed transiently or stably; (d) assaying the Cannabis plant or a cell thereof for a custom endonuclease-mediated modification in the DNA comprising or corresponding to or flanking the loci of any one of SEQ ID NO:13-646; and (e) identifying the Cannabis plant, a cell thereof, or a progeny cell thereof as comprising a modification in the loci of CsFPPS1, CsFPPS2, CsGPPS1 and/or CsGPPS2 genes.

It is a further object of the present invention to disclose the method as defined in any of the above, wherein the modified plant has reduced odor resulting from volatile organic compounds emission or is odor free or odorless Cannabis plant.

It is a further object of the present invention to disclose the method as defined in any of the above, further comprises steps of measuring or assaying the VOCs in the modified Cannabis plant using gas chromatography-mass spectrometry (GCMS) terpene profiling and quantitation techniques or by any other method for quantifying VOCs.

It is a further object of the present invention to disclose the method as defined in any of the above, wherein the VOCs are selected from essential oils, secondary metabolites, terpenoids, terpenes, oxygenated and any combination thereof.

It is a further object of the present invention to disclose the method as defined in any of the above, wherein the VOCs comprise at least one of hemiterpenes, monoterpenes, sesquiterpenes, diterpenes, sesterterpenes, triterpenes, tetraterpenes and polyterpenes.

It is a further object of the present invention to disclose the method as defined in any of the above, wherein the VOCs are selected from pinene, alpha-pinene, beta-pinene, cis-pinane, trans-pinane, cis-pinanol, trans-pinanol, limonene; linalool; myrcene; eucalyptol; a-phellandrene; b-phellandrene; a-ocimene; b-ocimene, cis-ocimene, ocimene, delta-3-carene; fenchol; sabinene, bomeol, isobomeol, camphene, camphor, phellandrene, a-phellandrene, a-terpinene, geraniol, linalool, nerol, menthol, terpinolene, a-terpinolene, b-terpinolene, g-terpinolene, delta-terpinolene, a-terpineol, trans-2-pinanol, caryophyllene, caryophyllene oxide, humulene, a-humulene, a-bisabolene; b-bisabolene; santalol; selinene; nerolidol, bisabolol; a-cedrene, b-cedrene, b-eudesmol, eudesm-7(11)-en-4-ol, selina-3,7(11)-diene, guaiol, valencene, a-guaiene, beta-guaiene, delta-guaiene, guaiene, famesene, a-famesene, b-famesene, elemene, a-elemene, b-elemene, gamma-elemene, delta-elemene, germacrene, germacrene A, germacrene B, germacrene C, germacrene D, germacrene E, oridonin, phytol, isophytol, ursolic acid, oleanolic acid, and/or 1.5 ene compounds, including guaia-1(10),11-diene, and 1.5 ene. Guaia-1(10), 11-diene.isoprene, α-pinene, β-pinene, d-limonene, β-phellandrene, α-terpinene, α-thujene, γ-terpinene, β-myrcene, (E)-β-ocimene, (−)-limonene, (+)-α-pinene, β-caryophyllene, and α-humulene and any combination thereof.

It is a further object of the present invention to disclose a modified Cannabis plant produced by the method as defined in any of the above.

It is a further object of the present invention to disclose a method for reducing or eliminating odor resulting from VOCs emission from a Cannabis plant, comprising steps of introducing into Cannabis plant cell, using targeted genome modification, at least one genomic modification conferring reduced expression or silencing of at least one gene involved in a terpene biosynthesis pathway.

It is a further object of the present invention to disclose the method as defined in any of the above, comprising steps of introducing using genome editing a loss of function mutation in at least one gene involved in a terpene biosynthesis pathway.

It is a further object of the present invention to disclose the method as defined in any of the above, wherein the method comprises steps of: (a) identifying at least one Cannabis gene involved in a terpene biosynthesis pathway; (b) designing and/or synthetizing at least one guide RNA (gRNA) comprising a nucleotide sequence corresponding or complementary to a target sequence is the at least one identified Cannabis gene involved in a terpene biosynthesis pathway; (c) transforming a Cannabis plant cells with endonuclease or nucleic acid encoding endonuclease, together with the at least one gRNA or a DNA encoding the gRNA; (d) optionally, culturing the transformed Cannabis cells; (e) selecting Cannabis plant or plant cells thereof carrying induced targeted loss of function mutation in the at least one gene involved in a terpene biosynthesis pathway; and (f) optionally, regenerating a modified Cannabis plant from the transformed plant cell, plant cell nucleus, or plant tissue.

It is a further object of the present invention to disclose the method as defined in any of the above, wherein the terpene biosynthesis pathway is selected from methylerythritol phosphate (MEP) pathway, mevalonic acid or mevalonate (MEV) pathway, isoprenoid biosynthetic pathway, formation of GPP, FPP and GGPP pathways, formation of squalene pathway, formation of Mono, Sesqui-und Di-Terpenes pathways, formation of triterpenes from squalene pathway and any combination thereof.

It is a further object of the present invention to disclose the method as defined in any of the above, wherein one gene involved in a terpene biosynthesis pathway is selected from CsTPS1PK, CsTPS4PK, CsTPS5PK, CsTPS6PK, CsTPS7PK, CsTPS8PK, CsTPS9PK, CsTPS10PK, CsTPS11PK, CsTPS12PK, CsTPS13PK, CsTPS14PK, CsTPS15PK, CsTPS16PK, CsTPS17PK, CsTPS18PK, CsTPS19PK, CsTPS20PK, CsTPS21PK, CsTPS22PK, CsTPS23PK, CsTPS24PK, CsTPS25PK, CsTPS26PK, CsTPS27PK, CsTPS30PK, CsTPS31PK, CsTPS32PK, CsTPS33PK, CsTPS34PK, CsTPS35PK, CsTPS12PK, CsTPS13PK, CsTPS1FN, CsTPS2FN, CsTPS3FN, CsTPS4FN, CsTPS5FN, CsTPS6FN, CsTPS7FN, CsTPS8FN, CsTPS9FN, CsTPS11FN, CsDXS1, CsDXS2, CsDXR, CsMCT, CsCMK, CsHDS, CsHDR, CsHMGS, CsHMGR1, CsHMGR2, CsMK, CsPMK, CsMPDC, CsIDI, CsFPPS1, CsFPPS2, CsGPPS1, CsGPPS2 and any combination thereof.

It is a further object of the present invention to disclose the method as defined in any of the above, wherein the gene involved in a terpene biosynthesis pathway is selected from (a) a gene encoding CsFPPS1 characterized by a sequence selected from SEQ ID NO: 1-3 or a functional variant thereof, (b) a gene encoding CsFPPS2 characterized by a sequence selected from SEQ ID NO: 4-6 or a functional variant thereof, (c) a gene encoding CsGPPS1 characterized by a sequence selected from SEQ ID NO: 7-9 or a functional variant thereof, (d) a gene encoding CsGPPS2 characterized by a sequence selected from SEQ ID NO: 10-12 or a functional variant thereof, and (e) any combination thereof.

It is a further object of the present invention to disclose the method as defined in any of the above, wherein the functional variant has at least 75% sequence identity to the gene sequence.

It is a further object of the present invention to disclose the method as defined in any of the above, wherein the gRNAs targeted for CsFPPS1, CsFPPS2, CsGPPS1 and CsGPPS2 comprising a SEQ ID NO: 13-237, SEQ ID NO: 238-390, SEQ ID NO: 391-530 and SEQ ID NO: 531-646, respectively.

It is a further object of the present invention to disclose a method for down regulation or silencing of Cannabis gene involved in a terpene biosynthesis pathway, which comprises utilizing the nucleotide sequence as set forth in at least one of SEQ ID NO:13-646 or a complementary sequence thereof, and any combination thereof, for introducing a targeted loss of function mutation into at least one of CsFPPS1, CsFPPS2, CsGPPS1 or CsGPPS2 gene, having genomic sequence comprising at least 80% identity to the sequence as set forth in SEQ ID NO:1, 4. 7 and 10 respectively using gene editing.

It is a further object of the present invention to disclose an isolated nucleic acid sequence having at least 75% sequence identity to a genomic sequence selected from the group consisting of SEQ ID NO:1, SEQ ID NO:4, SEQ ID NO:7 and SEQ ID NO:10.

It is a further object of the present invention to disclose an isolated nucleic acid sequence having at least 75% sequence identity to a coding sequence selected from the group consisting of SEQ ID NO:2, SEQ ID NO:5, SEQ ID NO:8 and SEQ ID NO:11.

It is a further object of the present invention to disclose an isolated amino acid sequence having at least 75% sequence similarity to amino acid sequence selected from the group consisting of SEQ ID NO:3, SEQ ID NO:6, SEQ ID NO:9 and SEQ ID NO:12.

It is a further object of the present invention to disclose an isolated nucleotide sequence having at least 75% sequence identity to a gRNA nucleotide sequence as set forth in SEQ ID NO:13-646.

It is a further object of the present invention to disclose a use of a nucleotide sequence as set forth in at least one of SEQ ID NO:13-646 and any combination thereof for silencing at least one gene involved in terpene biosynthesis pathway, by targeted gene editing of Cannabis CsFPPS1, CsFPPS2, CsGPPS1 or CsGPPS2 encoding genes.

### BRIEF DESCRIPTION OF THE FIGURES

Exemplary non-limited embodiments of the disclosed subject matter will be described, with reference to the following description of the embodiments, in conjunction with the figures. The figures are generally not shown to scale and any sizes are only meant to be exemplary and not necessarily limiting. Corresponding or like elements are optionally designated by the same numerals or letters.

FIG. 1A-D is photographically presenting various Cannabis tissues transformed with GUS reporter gene, where FIG. 4A shows axillary buds, FIG. 4B mature leaf, FIG. 4C calli, and FIG. 4D cotyledons;

FIG. 2 is photographically presenting PCR detection of transformed leaf tissue screened for the presence of the Cas9 gene two weeks post transformation; and

FIG. 3 is illustrating in vivo specific DNA cleavage by Cas9+gRNA (RNP) complex, as an embodiment of the present invention.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The present invention concerns a method of elimination of expression of terpene synthesis genes and thus creating odor free Cannabis plants.

It is an aim of the present invention to provide a novel method of effectively and consistently eliminating volatile compounds such as terpenes in a Cannabis plant. The method is based on gene editing of the Cannabis plant genome at specific nucleic acid sequences, which results in a set of desired traits such as odorless Cannabis plants.

The challenge here is to efficiently induce precise and predictable targeted point mutations pivotal to the terpene synthesis pathways in the Cannabis plant using the CRISPR/Cas9 system.

Without wishing to be bound by theory, it is acknowledged that a significant added value of gene editing is that it does not qualify as genetic modification so the resultant transgene-free plant will not be considered a GMO plant/product, at least in the USA (USDA, Washington, D.C., Mar. 28, 2018). While the exact and operational definition of genetically modified is debated and contested, it is generally agreed upon and accepted that genetic modification refers to plants and animals that have been altered in a way that wouldn't have arisen naturally through evolution. The clearest and most obvious example is a transgenic organism whose genome now incorporates a gene from another species inserted to confer a novel trait to that organism, such as pest resistance. The situation is different with genome editing, as the CRISPR machinery is not necessarily integrated into the plant genome, it is used transiently to create the desired mutation and only the editing event is inherited to the next generation.

Cannabis (Cannabis sativa) plants produce and accumulate a terpene-rich resin in glandular trichomes, which are abundant on the surface of the female inflorescence. Bouquets of different monoterpenes and sesquiterpenes are important components of Cannabis resin as they define some of the unique organoleptic properties and may also influence medicinal qualities of different Cannabis strains and varieties. Transcripts associated with terpene biosynthesis are highly expressed in trichomes compared to non-resin producing tissues.

The present invention disclosed herein provides a method for producing a plant with decreased organic volatile compounds (VOCs) and more specifically terpene molecules when compared to a corresponding wild type, non-edited Cannabis plant. According to some aspects, the present invention provides plant, plant cell or its derivatives exhibiting decreased levels of terpene synthesis genes achieved by gene-editing, and plants comprised of such cells, progeny, seed and pollen derived from such plants, and methods of making and methods of using such plant cell(s) or plant(s), progeny, seed(s) or pollen. Particularly, said improved trait(s) are manifested by decreased expression of terpene synthesis genes resulting in lower volatile molecules such as terpenes. Preferably, the desirable trait(s) are achieved via knocking out by genome editing the Geranyl diphosphate synthase (GPPS) and Farnesyl diphosphate synthase (FPPS) genes, whereby the decreased expression of terpene synthesis genes reduces and/or eliminates the odor emitted by the Cannabis plant.

According to one embodiment, the present invention provides a modified Cannabis plant exhibiting reduced volatile organic compounds (VOCs) emission, wherein said modified plant comprises at least one targeted gene modification conferring reduced expression or silencing of at least one gene involved in a terpene biosynthesis pathway.

The present invention further provides a method for producing a modified Cannabis plant exhibiting reduced volatile organic compounds (VOCs) emission, said method comprises introducing into Cannabis plant cell, using targeted genome modification, at least one genomic modification conferring reduced expression or silencing of at least one gene involved in a terpene biosynthesis pathway.

It is further within the scope to provide a method for reducing or eliminating odor resulting from VOCs emission from a Cannabis plant, comprising steps of introducing into Cannabis plant cell, using targeted genome modification, at least one genomic modification conferring reduced expression or silencing of at least one gene involved in a terpene biosynthesis pathway.

Other main aspects of the present invention include a method for down regulation or silencing of Cannabis gene involved in a terpene biosynthesis pathway, which comprises utilizing the nucleotide sequence as set forth in at least one of SEQ ID NO:13-646 or a complementary sequence thereof, and any combination thereof, for introducing a targeted loss of function mutation into at least one of CsFPPS1, CsFPPS2, CsGPPS1 or CsGPPS2 gene, having genomic sequence comprising at least 80% identity to the sequence as set forth in SEQ ID NO:1, 4. 7 and 10 respectively using gene editing.

The present invention further provides an isolated nucleic acid and/or amino acid sequence having at least 75% sequence identity to a sequence selected from the group consisting of SEQ ID NO:1-SEQ ID NO:646 and any combination thereof.

It is also within the scope to provide use of a nucleotide sequence as set forth in at least one of SEQ ID NO:13-646 and any combination thereof for silencing at least one gene involved in terpene biosynthesis pathway, by targeted gene editing of Cannabis CsFPPS1, CsFPPS2, CsGPPS1 or CsGPPS2 encoding genes.

### Reference is Now Made to Volatile Organic Compounds Definitions

It is commonly known that the characteristic smell of Cannabis is primarily the result of a class of small volatile organic molecules known as terpenes. Terpenes are a primary constituent of the essential oil extract of Cannabis. Therefore, the disclosed embodiments provide a Cannabis plant and any product thereof that is produced by removing or reducing the naturally occurring compliment of volatile organic molecules from Cannabis by gene editing of terpene biosynthesis genes. At least 200 terpenes are found in the Cannabis plant but 14 are commonly found in significant quantities, which vary in quantity depending on the strain of the Cannabis plant. These common terpenes may include, isoprene, α-pinene, β-pinene, Δ3-carene, d-limonene, camphene, myrcene, β-phellandrene, sabinene, α-terpinene, ocimene, α-thujene, terpinolene and γ-terpinene.

It is acknowledged that terpenes are synthesized by the enzyme terpene synthase.

As used herein, the term “terpene” refers to a class of compounds that consist of one or more isoprene units. Terpenes may be linear (acyclic) or contain rings. A terpene containing oxygen functionality or missing a methyl group is referred to herein as a terpenoid. Terpenoids fall under the class of terpenes as used herein.

Terpenes are biosynthetically produced from units of isoprene, which has the basic molecular formula C5H8. The molecular formula of terpenes is a multiple of that molecular formula, (C5H8)n where n is the number of linked isoprene residues. The resulting terpenes are classified consecutively according to their size as hemiterpenes, monoterpenes, sesquiterpenes, diterpenes, sesterterpenes, triterpenes, tetraterpenes and polyterpenes.

Depending on the number of C5 units and possible substitutions, they are further classified based on number of units (e.g., C10 monoterpenes, two subunits, C15, sesquiterpenes, and three subunits) or functional groups (terpenoids and oxygenated). It is noted that mono- and sesquiterpenes are classified as volatile and semi-volatile compounds, respectively, and higher order terpenes (e.g., C20 diterpenes and C30 triterpenes) exist as steroids, waxes, and resins.

According to an embodiment of the present invention, Cannabis mono- and sesquiterpenes are responsible for the characteristic smell of the plant and its products.

The methods described herein are useful in reducing odor produced by a terpene by silencing using genome editing a gene involved in the terpene synthesis pathway.

As used herein, the term “reduce” is defined as the ability to reduce the likelihood of detecting the odor produced by the terpene (or VOCs emission) up to about 50%, up to about 60%, up to about 70%, up to about 80%, up to about 90%, up to about 95%, or up to about 99% when compared to not using the methods as described herein. As used herein, the term “reduce” is also defined as the ability to completely eliminate the likelihood of detecting the odor produced by the terpene when compared to not using the methods as described herein. The methods described herein are useful in reducing the odor produced by hemiterpenes, monoterpenes, sesquiterpenes, diterpenes, sesterpenes, triterpenes, tetraterpenes, or polyterpenes.

The methods described herein reduce the odor produced by a plurality of (i.e., two or more) of terpenes. It is understood that each terpene produces a distinct odor. The methods described herein reduce the odor produced collectively by the plurality of terpenes.

Non limiting examples of terpene biosynthetic pathway enzyme is limonene synthase, squalene synthase, phytoene synthase, myrcene synthase, germacrene D synthase, a-farnesene synthase, or geranyllinalool synthase.

According to some aspects of the present invention, the gene involved in a terpene biosynthesis pathway is selected from a gene encoding Cannabis farnesyl diphosphate (FPP) synthase1 (CsFPPS1), Cannabis farnesyl diphosphate (FPP) synthase2 (CsFPPS2), Cannabis Geranyl diphosphate (GPP) synthase1 (CsGPPS1), Cannabis Geranyl diphosphate (GPP) synthase2 (CsGPPS2) and any combination thereof.

Cannabis terpene synthase (TPS) promoters or biologically active fragments thereof that may be used to genetically manipulate the synthesis of terpenes (e.g. monoterpenes such as a-pinene, b-pinene, myrcene, limonene, b-ocimene, and terpinolene, and sesquiterpenes such as b-caryophyllene, bergamotene, famesene, a-humulene, alloaromadendrene, and d-selinene) may be further used to eliminate gene involved in a terpene biosynthesis pathway using gene editing.

This can for example be accomplished by:

a) deletion of the entire gene encoding the gene involved in a terpene biosynthesis pathway; or

b) deletion of the entire coding region encoding the gene involved in a terpene biosynthesis pathway; or

c) deletion of part of the gene encoding the gene involved in a terpene biosynthesis pathway leading to a total loss of the endogenous activity of the enzyme.

Reference is now made to gene editing techniques used in the present invention.

Mutation breeding refers to a host of techniques designed to rapidly and effectively induce desired or remove unwanted/undesirable traits via artificial mutations in a target organism. Gene editing is such a mutation breeding tool which offers significant advantages over genetic modification. Genetic modification is a molecular technology involving inserting a DNA sequence of interest, coding for a desirable trait, into an organism's genome. Gene editing is a mutation breeding tool which allows precise modification of the genome. In this tool/mechanism, a DNA nuclease (a protein complex from the Cas family) is precisely directed toward an exact (target) genome locus using a guide RNA, and then it cleaves the genome at that target site.

One advantage of using the CRISPR/Cas system over other genetic modification approaches is that Cas family proteins are easily programmed to make a DNA double strand break (DSB) at any desired loci. The initial cleavage is followed by repairing chromosomal DSBs. Without wishing to be bound by theory, there are two major cellular repair pathways in that respect: Non-homologous end joining (NHEJ) and Homology directed repair (HDR). According to one embodiment, the present invention concerns usage of NHEJ, which is active throughout the cell cycle and has a higher capacity for repair, as there is no requirement for a repair template (e.g. sister chromatid or homologue) or extensive DNA synthesis. NHEJ also capable of completing repair of most types of breaks in tens of minutes—an order of magnitude faster than HDR. It is further acknowledged that NHEJ-mediated repair of DSBs is useful in cases where making a null allele (knockout) in a gene of interest is desirable, as it is prone to generating indel errors. It is noted that indel errors generated in the course of repair by NHEJ are typically small (1-10 bp) but are heterogeneous. There is consequently a relatively high chance of causing a frameshift mutation by using this pathway. The deletion can be less heterogeneous when constrained by sequence identities in flanking sequence (microhomologies).

Additionally, there is no foreign DNA left over in the plant after selection for plants, which contain the desired editing event and do not carry the CRISPR/Cas machinery. This significant advantage has allowed gene editing to be viewed by many legal systems around the world as GMO-free.

Advances made recently in an attempt to more efficiently target and cleave genomic DNA by site specific nucleases [e.g. zinc finger nucleases (ZFNs), meganucleases, transcription activator-like effector nucleases (TALENS)] are also encompassed within the scope of the present invention. For example, it is acknowledged that RNA-guided endonucleases (RGENs) have been introduced, and they are directed to their target sites by a complementary RNA molecule. These systems, included within the scope of the present invention, have a DNA-binding domain that localizes the nuclease to a target site. The site is then cut by the nuclease. According to aspects of the present invention, these systems are used to induce targeted mutagenesis, induce targeted deletions of cellular DNA sequences, and facilitate targeted recombination of an exogenous donor DNA polynucleotide within a predetermined genomic locus.

According to one embodiment, RGEN used in the present invention is Clustered Regularly Interspaced Short Palindromic Repeats/CRISPR-associated nuclease (CRISPR/Cas) with an engineered crRNA/tracr RNA. CRISPR/Cas9 are cognates that find each other on the target DNA. The CRISPR-Cas9 system is a tool of choice in gene editing because it is faster, cheaper, more accurate, and more efficient than other available RGENs. A small fragment of RNA with a short “guide” sequence (gRNA) is created that binds to a specific target sequence of DNA in a genome. The RNA also binds to the Cas9 enzyme. The modified RNA is used to recognize the DNA sequence, and the Cas9 enzyme cuts the DNA at the targeted location. Although Cas9 is the enzyme that is used most often, other enzymes (for example Cpf1) can also be used. Once the DNA is cut, the cell's own DNA repair machinery add or delete pieces or fragments of genetic material resulting in mutation.

According to further embodiments of the present invention, ribonucleoprotein protein complex (RNP) is used. Ribonucleoprotein protein complex is formed when a Cas protein is incubated with gRNA molecules and then transformed into cells for inducing editing events in the cell. According to one embodiment of the present invention, RNP's can be delivered using biolistics.

Reference is now made to the biolistics method for transforming Cannabis plants and cells thereof.

Biolistics is a method for the delivery of nucleic acid and or proteins to cells by high-speed particle bombardment. The technique uses a pressurized gun (gene gun) to forcibly propel a payload comprised of an elemental particle of a heavy metal coated with plasmid DNA to transform plant cellular organelles. After the DNA-carrying vector has been delivered, the DNA is used as a template for transcription and sometimes it integrates into a plant chromosome (“stable” transformation). If the vector also delivered a selectable marker, then stably transformed cells can be selected and cultured. Transformed plants can become totipotent and even display novel and heritable phenotypes.

According to further aspects of the present invention, the skeletal biolistic vector design includes not only the desired gene to be inserted into the cell, but also promoter and terminator sequences as well as a reporter gene used to enable the ensuing detection and removal cells which failed to incorporate the exogenous DNA.

It is this herein noted that in addition to DNA, the use of a Cas9 protein and a gRNA molecule is used for biolistic delivery. The advantage of using a protein and a RNA molecule is that the complex initiates editing upon reaching the cell nucleus. Without wishing to be bound by theory, when using DNA for editing, the DNA first has to be transcribed in the nucleus; but when using RNA for editing, RNA is translated already in the cytoplasm. This forces the Cas protein to shuttle back to the nucleus, find the relevant guides and only then can editing be achieved.

As used herein, the term “CRISPR” refers to an acronym that means Clustered Regularly Interspaced Short Palindromic Repeats of DNA sequences. CRISPR is a series of repeated DNA sequences with unique DNA sequences in between the repeats. RNA transcribed from the unique strands of DNA serves as guides for directing cleaving. CRISPR is used as a gene editing tool. In one embodiment, CRISPR is used in conjunction with (but not limited to) Cpf1, Cas9, Cas12, Cas13, Cas14, CasX or CasY.

As used herein, the term “transformation” refers to the deliberate insertion of genetic material into plant cells. In one embodiment transformation is executed using, but not limited to, bacteria and/or virus. In another embodiment, transformation is executed via biolistics using, but not limited to, DNA or RNPs.

As used herein, the term “Cas” refers to CRISPR associated proteins that act as enzymes cutting the genome at specific sequences. Cas9 refers to a specific group of proteins known in the art. RNA molecules direct various classes of Cas enzymes to cut a certain sequence found in the genome. In one embodiment, the CRISPR/Cas9 system cleaves one or two chromosomal strands at known DNA sequence. In a further embodiment, one of the two chromosomal strands is mutated. In one embodiment, two of the two chromosomal strands are mutated.

As used herein, the term “chromosomal strand” refers to a sequence of DNA within the chromosome. When the CRISPR/Cas9 system cleaves the chromosomal strands, the strands are cut leaving the possibility of one or two strands being mutated, either the template strand or coding strand.

As used herein, the term “PAM” (protospacer adjacent motif) refers to a targeting component of the transformation expression cassette which is a very short (2-6 base pair) DNA sequence immediately following the DNA sequence targeted by the Cas9 nuclease in the CRISPR system.

Within the context of this disclosure, other examples of endonuclease enzymes include, but are not limited to, Cpf1, Cas9, Cas12, Cas13, Cas14, CasX or CasY.

According to some aspects, the entire invention is relevant to any of the terpene synthesis genes in the Cannabis plant, and not limited only to the genes listed in Tables 5 and 6. The method of identifying the specific gRNA sequences for each terpene gene paired with a specific complementary PAMs, and/or characterization of a plurality of gRNAs directing the CRISPR/Cas system to cleave chromosomal strands coding for those various genes is similar or identical to the method described in the current disclosure for the CsGPPS1, CsGPPS2, CsFPPS1 & CsFPPS2 genes. Non-limiting examples of terpene genes relevant to this invention are listed in Tables 5 and 6.

Reference is now made to analysis of terpene and terpenoid content in Cannabis biomass.

It is included within the scope that an exemplified, not limiting method that may be used by the present invention, amongst other methods known to the skilled person is the method described in Krill et al, 2020, incorporated herein by its entirety by reference. In summary, the method is based on hexane extract from Cannabis biomass, with dodecane as internal standard, and a gradient. The method can detect about 50 individual terpenes and terpenoids. The validation parameters of the method are comparable to other commonly known studies. This high-throughput gas chromatography-mass spectrometry (GCMS) terpene profiling method enable to quantify terpenes in medicinal cannabis biomass, such as the modified Cannabis plant of the present invention.

According to one embodiment, for sampling, dried samples of Cannabis inflorescence may be used.

The method enable accurately measuring the non-cannabinoid content in cannabis, particularly terpenes and terpenoids, in large scale.

According to one embodiment, the present invention provides a modified Cannabis plant exhibiting reduced volatile organic compounds (VOCs) emission, wherein said modified plant comprises at least one targeted gene modification conferring reduced expression or silencing of at least one gene involved in a terpene biosynthesis pathway.

According to a further embodiment, the present invention provides a method for reducing or eliminating odor resulting from volatile compounds, more specifically terpenes, in Cannabis plants (e.g. C. sativa, C. indica, C. ruderlis). The method comprises steps of;

a) selecting and identifying a gene involved in a terpene synthesis pathway of a Cannabis species;

b) synthesizing or designing a gRNA corresponding to a targeted cleavage region in the identified gene locus within the Cannabis genome;

c) transforming into the Cannabis plant or a cell thereof endonuclease or nucleic acid encoding endonuclease (e.g. CRISPR/Cas9 system), together with the synthesized gRNA or a DNA encoding the gRNA;

d) culturing the transformed Cannabis plant cells;

e) selecting the Cannabis cells which express desired mutations in the editing target region, and

f) regenerating a plant from said transformed plant cell, plant cell nucleus, or plant tissue.

It is further within the scope that the identified gene is a gene involved in the terpene biosynthesis pathways of Cannabis, such a gene may be selected from the group comprising CsFPPS1, CsFPPS2, CsGPPS1 and CsGPPS2, characterized by a sequence as set forth in any of SEQ ID NO: 1-12.

According to a further embodiment the gRNAs targeted for CsFPPS1, CsFPPS2, CsGPPS1 and CsGPPS2 comprising a SEQ ID NO: 13-237, SEQ ID NO: 238-390, SEQ ID NO: 391-530 and SEQ ID NO: 531-646, respectively.

According to further aspects of the present invention, the target domain sequence within the Cannabis genome is selected from the group comprising of 1) a nucleic acid sequence encoding the polypeptide of CsFPPS1, the nucleic acid having a sequence as set forth in SEQ ID NO: 1 (2) a nucleic acid sequence encoding the polypeptide of CsFPPS2, the nucleic acid having a sequence as set forth in SEQ ID NO: 4 (3) a nucleic acid sequence encoding the polypeptide of CsGPPS1, the nucleic acid having a sequence as set forth in SEQ ID NO: 7 (4) a nucleic acid sequence encoding the polypeptide of CsGPPS2, the nucleic acid having a sequence as set forth in SEQ ID NO: 10 (5) a nucleic acid sequence having at least 80% sequence identity to at least 200 contiguous nucleotides of the nucleic acid sequence of CsFPPS1, (6) a nucleic acid sequence having at least 80% sequence identity to at least 200 contiguous nucleotides of the nucleic acid sequence of CsFPPS2, (7) a nucleic acid sequence having at least 80% sequence identity to at least 200 contiguous nucleotides of the nucleic acid sequence of CsGPPS1, (8) a nucleic acid sequence having at least 80% sequence identity to at least 200 contiguous nucleotides of the nucleic acid sequence of CsGPPS2,

It is further within the scope of the current invention that the transformation into Cannabis plant cell is carried out using Agrobacterium to deliver an expression cassette comprising a) a selection marker, b) a nucleotide sequence encoding one or more gRNA molecules comprising a DNA sequence which is complementary with a target domain sequence within a gene selected from CsFPPS1, CsFPPS2, CsGPPS1 and CsGPPS2, c) a nucleotide sequence encoding a Cas molecule from, but not limited to, Streptococcus pyogenes and/or Staphylococcus aureus (PAM sequences NGG and NNGRRT respectively). Other optional PAM include, NNNNGATT (NmeCas9), NNAGAAW (StCas9), NAAAAC (TdCas9), NNGRRT (SaCas9) and TBN (Cas-phi).

The method of the present invention further comprises introducing into a Cannabis plant cell a nucleic acid composition comprising: a) a first nucleotide sequence encoding the targeted gRNA molecule and b) a second nucleotide sequence encoding the Cas molecule.

According to other aspects the method of the present invention comprises introduction into a Cannabis plant cell a construct comprising (a) Cas DNA and gRNA sequence selected from the group consisting of SEQ ID NO:13-646 and any combination thereof, or (b) a ribonucleoprotein (RNP) complex comprising Cas protein and gRNA sequence selected from the group consisting of SEQ ID NO:13-646 and any combination thereof.

It is further within the scope of the current invention that the CRISPR/Cas system is delivered to the Cannabis cell by a plant virus.

According to a further embodiment of the present invention, the Cas protein is selected from the group comprising but not limited to Cpf1, Cas9, Cas12, Cas13, Cas14, CasX or CasY.

It is also within the scope to provide a method for increasing Cannabis yield comprising steps of:

(a) introducing into a Cannabis plant or a cell thereof (i) at least one RNA-guided endonuclease comprising at least one nuclear localization signal, or a nucleic acid encoding at least one RNA-guided endonuclease comprising at least one nuclear localization signal, (ii) at least one guide RNA or DNA encoding at least one guide RNA, and, optionally, (iii) at least one donor polynucleotide; and

(b) culturing the Cannabis plant or cell thereof such that each guide RNA directs an RNA-guided endonuclease to a targeted site in the chromosomal sequence where the RNA-guided endonuclease introduces a double-stranded break in the targeted site, and the double-stranded break is repaired by a DNA repair process such that the chromosomal sequence is modified, wherein the targeted site is located in the CsFPPS1, CsFPPS2, CsGPPS1 and/or CsGPPS2 genes and the chromosomal modification interrupts or interferes with transcription and/or translation of the CsFPPS1, CsFPPS2, CsGPPS1 and/or CsGPPS2 genes.

It is also within the scope of the current invention that the RNA-guided endonuclease is derived from a clustered regularly interspersed short palindromic repeats (CRISPR)/CRISPR-associated (Cas) system.

According to a further embodiment of the present invention, the editing of CsFPPS1, CsFPPS2, CsGPPS1 and/or CsGPPS2 genes does not insert exogenous genetic material and produces a non-naturally occurring Cannabis plant or cell thereof.

According to further aspects, the method of silencing Cannabis terpene synthesis of the present invention comprises steps of:

(a) identifying at least one locus within a DNA sequence in a Cannabis plant or a cell thereof for CsFPPS1, CsFPPS2, CsGPPS1 and/or CsGPPS2 genes;

(b) identifying at least one custom endonuclease recognition sequence within the at least one locus of CsFPPS1, CsFPPS2, CsGPPS1 and/or CsGPPS2 genes; and

(e) identifying the Cannabis plant, a cell thereof, or a progeny cell thereof as comprising a modification in the loci of CsFPPS1, CsFPPS2, CsGPPS1 and/or CsGPPS2 genes.

It is further within the scope of the present invention to provide a transgenic Cannabis plant produced by the method as defined in any of the above.

According to a further aspect, the method of the present invention further comprises editing of genes involved in the terpene synthesis pathway listed in Table 6.

The present invention further provides a method of editing the genes listed in Table 6, e.g. in the same manner described for the genes encoding CsFPPS1, CsFPPS2, CsGPPS1 and/or CsGPPS2, namely, but not limited to, identifying specific gRNA sequences for each of the genes of Table 6, and constructing specific gRNAs for targeting regions in each of the genes to thereby silence each of the individual genes by using gene editing technology as described above.

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects, it means having or participating in the same relationship (such as type or species, kind, degree, position, correspondence, or function). It further means related or accompanying. In some embodiments of the present invention, it refers to plants of the same Cannabis species, strain, or variety or to sibling plant, or one or more individuals having one or both parents in common.

A “plant” as used herein refers to any plant at any stage of development, particularly a seed plant. The term “plant” includes the whole plant or any parts or derivatives thereof, such as plant cells, seeds, plant protoplasts, plant cell tissue culture from which tomato plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

It is further within the scope that the term “plant” includes a whole plant and any descendant, cell, tissue, or part of a plant. The term “plant parts” include any part (s) of a plant, including, for example and without limitation: seed; a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. It is noted that some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

According to further aspects of the present invention, plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root.

The term “plant cell” used herein refers to a structural and physiological unit of a plant, comprising a protoplast and a cell wall. The plant cell may be in a form of an isolated single cell or an aggregate of cells (e.g., a friable callus and a cultured cell), or as a part of higher organized unit such as, for example, plant tissue, a plant organ, or a whole plant. Thus, a plant cell may be a protoplast, a gamete-producing cell, or a cell or collection of cells that can regenerate into a whole plant. As such, a seed, which comprises multiple plant cells and is capable of regenerating into a whole plant, is considered a “plant cell” in embodiments herein.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as a root, stem, leaf, flower, flower bud, or embryo.

The term “Plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

The term “protoplast” as used herein, refers to a plant cell that had its cell wall completely or partially removed, with the lipid bilayer membrane thereof naked, and thus includes protoplasts, which have their cell wall entirely removed, and spheroplasts, which have their cell wall only partially removed, but is not limited thereto. Typically, a protoplast is an isolated plant cell without cell walls, which has the potency for regeneration into cell culture or a whole plant.

As used herein, the term “progeny” or “progenies” refers in a non-limiting manner to any subsequent generation of the plant, including offspring or descendant plants. According to certain embodiments, the term “progeny” or “progenies” refers to plants developed, grown, or produced from the disclosed or deposited seeds as detailed inter alia. The grown plants preferably have the desired traits of the disclosed or deposited seeds, i.e. eliminated expression of at least one terpene synthesis gene, e.g. encoding CsFPPS1, CsFPPS2, CsGPPS1 and/or CsGPPS2 producing odorless Cannabis plant.

The term “Cannabis” refers hereinafter to a genus of flowering plants in the family Cannabaceae. Cannabis is an annual, dioecious, flowering herb that includes, but is not limited to three different species, Cannabis sativa, Cannabis indica and Cannabis ruderalis. The term also refers to hemp. Cannabis plants produce a group of chemicals called cannabinoids. Cannabinoids, terpenoids, and other compounds are secreted by glandular trichomes that occur most abundantly on the floral calyxes and bracts of female Cannabis plants.

As used herein, Cannabis includes any plant or plant material derived from a Cannabis plant (i.e., Cannabis sativa, Cannabis indica and Cannabis ruderalis), naturally or through selective breeding or genetic engineering. The Cannabis may be used for therapeutic, medicinal, research, recreational purposes or any yet unforeseen purpose. Ways for consuming the Cannabis plant of the present invention or products thereof according to embodiments may include, but are not limited to, inhalation by smoking dried Cannabis plant material, inhalation by smoking Cannabis plant extracts or by ingesting Cannabis plant material or plant extracts such as, for example, in the form of edible Cannabis products that incorporate raw plant material, where potentially undesirable odor has been removed by the method of the present invention. For purposes of this disclosure, the disclosed embodiments will be described with respect to the production of a modified form of Cannabis plant material It will be understood that the disclosed products and methods may apply to all types, forms and uses of Cannabis.

According to some aspects, Marijuana includes all varieties of the Cannabis genus that contain substantial amounts of THC. As used herein, Hemp includes all varieties of the Cannabis genus that contain negligible amounts of THC. Hemp specifically includes the plant Cannabis sativa L. and any part of that plant, including the seeds thereof and all derivatives with a THC concentration defined according to relevant regulations.

The term “odor” as used herein encompass an odor (American English) or odour (British English) and generally refers to a quality of something that stimulates the olfactory organ, e.g. scent or a sensation resulting from adequate stimulation of the olfactory organ, e.g. smell. It is caused by one or more volatilized chemical compounds that are generally found in low concentrations that humans and animals can perceive by their sense of smell. An odor is also called a “smell” or a “scent”, which can refer to either a pleasant or an unpleasant odor. In the context of the present invention, it means odor-producing emissions associated with Cannabis production facilities. The characteristic odor associated with Cannabis is attributed to the release of chemical compounds into the air known as volatile organic compounds (VOCs). Over 200 different VOCs have been identified from packaged cannabis samples. VOCs responsible for the aroma profiles may be different due to different rates of chemical volatilization. One approach used for characterizing odor mixtures is the use of the odor unit, which is the ratio between the amount of odorant present in a volume of a neutral (odorless) gas at the odor detection threshold of the odor evaluation panelists. For example, the odor unit is used by the Ontario Ministry of Agriculture, Food and Rural Affairs to categorize odors under the Nutrient Management Act and by the Ontario Ministry of the Environment and Climate Change to determine the compliance of industrial facilities with regulations under the Environmental Protection Act. Exposure to unpleasant odors may affect an individual's quality of life and sense of well-being. Exposure to odorous compounds can potentially trigger physical symptoms, depending on the type of substance responsible for the odor, the intensity of the odor, the frequency of the odor, the duration of the exposure, and the sensitivity of the individual detecting the odor.

The term “genome” as applies to plant cells, encompasses chromosomal DNA found within the nucleus, and organelle DNA found within subcellular components (e.g., mitochondrial, plastid) of the cell.

A “genetically modified plant” includes, in the context of the present invention, a plant which comprises within its genome an exogenous polynucleotide. For example, the exogenous polynucleotide is stably integrated within the genome such that the polynucleotide is passed on to successive generations. The exogenous polynucleotide may be integrated into the genome alone or as part of a recombinant DNA construct. The modified gene or expression regulatory sequence means that, in the plant genome, comprises one or more nucleotide substitution, deletion, or addition. For example, a genetically modified plant obtained by the present invention may contain an insertion, deletion or nucleotide substitution relative to the wild type plant (corresponding plant that is not genetically modified).

As used herein, the term “exogenous” with respect to sequence, means a sequence that originates from a foreign species, or, if from the same species, is substantially modified from its native form in composition and/or genomic locus by deliberate human intervention.

As used herein the term “genetic modification” refers hereinafter to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified Cannabis plants with improved domestication traits are generated using genome editing mechanism. This technique enables to achieve in planta modification of specific genes that relate to and/or control the terpene biosynthesis in the Cannabis plant.

The term “genome editing”, or “gene editing”, or “genome/genetic modification”, or “genome engineering” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations.

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

Reference is now made to exemplary genome editing terms used by the current disclosure:

### Genome Editing Glossary

| | |
|---|---|
| Cas = CRISPR-associated genes | Indel = insertion and/or deletion |
| Cas9, Csn1 = a CRISPR-associated protein | NHEJ = Non-Homologous End Joining |
| containing two nuclease domains, that is | PAM = Protospacer-Adjacent Motif |
| programmed by small RNAs to cleave DNA | RuvC = an endonuclease domain named for |
| crRNA = CRISPR RNA | an E. coli protein involved in DNA repair |
| dCAS9 = nuclease-deficient Cas9 | sgRNA = single guide RNA |
| DSB = Double-Stranded Break | tracrRNA, trRNA = trans-activating crRNA |
| gRNA = guide RNA | TALEN = Transcription-Activator Like |
| HDR = Homology-Directed Repair | Effector Nuclease |
| HNH = an endonuclease domain named | ZFN - Zinc-Finger Nuclease |
| for characteristic histidine and asparagine | |
| residues | |

According to specific aspects of the present invention, the CRISPR (Clustered Regularly Interspaced Short Palindromic Repeats) and CRISPR-associated (Cas) genes are used for the first time for generating genome modification in targeted genes in the Cannabis plant. It is herein acknowledged that the functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

The terms “Cas9 nuclease” and “Cas9” or CRISPR/Cas can be used interchangeably herein, and refer to a RNA directed nuclease, including the Cas9 protein or fragments thereof (such as a protein comprising an active DNA cleavage domain of Cas9 and/or a gRNA binding domain of Cas9). Cas9 is a component of the CRISPR/Cas (clustered regularly interspaced short palindromic repeats and its associated system) genome editing system, which targets and cleaves a DNA target sequence to form a DNA double strand breaks (DSB) under the guidance of a guide RNA.

According to further aspects of the invention, Cas protein, such as Cas9 (also known as Csn1) participates in the processing of crRNAs, and is responsible for the destruction of the target DNA. Cas9's function in both of these steps relies on the presence of two nuclease domains, a RuvC-like nuclease domain located at the amino terminus and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand to gRNA, while the RuvC domain cleaves the non-complementary strand.

It is further noted that the double-stranded endonuclease activity of Cas9 also requires that a short-conserved sequence, (2-5 nts) known as protospacer-associated motif (PAM), follows immediately 3′-of the crRNA complementary sequence.

According to further aspects of the invention, a two-component system may be used by the current invention, combining trRNA and crRNA into a single synthetic single guide RNA (sgRNA) for guiding targeted gene alterations.

A general exemplified CRISPR/Cas9 mechanism of action is depicted by Xie, Kabin, and Yinong Yang. “RNA-guided genome editing in plants using a CRISPR-Cas system.” Molecular plant 6.6 (2013): 1975-1983. As shown in this publication, which is incorporated herein by reference, the Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and an NGG motif (called protospacer-adjacent motif or PAM) that follows the base-pairing region in the complementary strand of the targeted DNA.

As the DNA-cutting such as CRISPR-Cas9 and related genome-editing tools mainly originate from bacteria, Cas proteins apparently evolving in viruses that infect bacteria, are also within the scope of the present invention. For example, the most compact Cas variants were found in bacteriophages (bacteria-infecting viruses) and they herein referred to as CasΦ (Cas-phi).

It is therefore within the scope of the present invention that the nuclease used for base-editing of a predetermined Cannabis HR-related gene may be selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi) and any combination thereof.

The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “guide RNA” or “gRNA” can be used interchangeably herein, and are composed of crRNA and tracrRNA molecules forming complexes through partial complement, wherein crRNA comprises a sequence that is sufficiently complementary to a target sequence for hybridization and directs the CRISPR complex (Cas9+crRNA+tracrRNA) to specifically bind to the target sequence. It is herein acknowledged and within the scope, that single guide RNA (sgRNA) can be designed, which comprises the characteristics of both crRNA and tracrRNA.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component, which distinguishes bacterial self from non-self DNA, thereby preventing the CRISPR locus from being targeted and destroyed by nuclease.

The term “deaminase” as used herein refers to an enzyme that catalyzes the deamination reaction. In some embodiments of the present invention, the deaminase refers to a cytidine deaminase, which catalyzes the deamination of a cytidine or a deoxycytidine to a uracil or a deoxyuridine, respectively. In some other embodiments of the present invention, it refers to adenine deaminase. This enzyme catalyzes the hydrolytic deamination of adenosine to form inosine and deoxyadenosine to deoxyinosine.

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

The term “microRNAs” or “miRNAs” refers hereinafter to small non-coding RNAs that have been found in most of the eukaryotic organisms. They are involved in the regulation of gene expression at the post-transcriptional level in a sequence specific manner. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “in planta” means in the context of the present invention within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

The term “introduction” or “introduced” referring to a nucleic acid molecule (such as a plasmid, a linear nucleic acid fragment, RNA etc.) or protein into a plant means transforming the plant cell with the nucleic acid or protein so that the nucleic acid or protein can function in the plant cell.

As used herein, the term “transformation” includes stable transformation and transient transformation.

“Stable transformation” refers to introducing an exogenous nucleotide sequence into a plant genome, resulting in genetically stable inheritance. Once stably transformed, the exogenous nucleic acid sequence is stably integrated into the genome of the plant and any successive generations thereof.

“Transient transformation” refers to introducing a nucleic acid molecule or protein into a plant cell, performing its function without stable inheritance. In transient transformation, the exogenous nucleic acid sequence is not integrated into the plant genome.

The term “orthologue” as used herein refers hereinafter to one of two or more homologous gene sequences found in different species.

The term “functional variant” or “functional variant of a nucleic acid or amino acid sequence” as used herein, for example with reference to SEQ ID NOs: 1-12 refers to a variant of a sequence or part of a sequence which retains the biological function of the full non-variant allele (e.g. CsFPPS1, CsFPPS2, CsGPPS1 & CsGPPS2 wild type allele) and hence has the activity of CsFPPS1, CsFPPS2, CsGPPS1 & CsGPPS2 expressed gene or protein. A functional variant also comprises a variant of the gene of interest encoding a polypeptide, which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mat are identical or associated with a single gene, or multiple genes, or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele. In the context of the current invention, the term allele refers to the herein identified gene sequences in Cannabis encoding terpene synthesis proteins, namely CsFPPS1, CsFPPS2, CsGPPS1 & CsGPPS2 having the genomic nucleotide sequence as set forth in SEQ ID NOs: 1, 4, 7 and 10 respectively; coding sequence (CDS) as set forth in SEQ ID NOs: 2, 5, 8 and 11 respectively; and amino acid sequence as set forth in SEQ ID NOs: 3, 6, 9 and 12 respectively.

As used herein, the term “locus” (loci plural) means a specific place or places or region or a site on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

In specific embodiments, the Cannabis plants of the present invention comprise homozygous configuration of at least one of the mutated genes encoding CsFPPS1, CsFPPS2, CsGPPS1 & CsGPPS2, said mutated genes or variants eliminate odor emission from the Cannabis plant.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters, which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments, which identify the most similar region between two sequences. For domains that are found in different sequence contexts in different proteins, the alignment should be limited to the homologous domain, since the domain homology is providing the sequence similarity captured in the score. According to some aspects, the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids, which may not be adjacent.

As used herein, the terms “nucleic acid”, “nucleic acid sequence”, “nucleotide”, “nucleic acid molecule” “nucleic acid fragment” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene.

The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA. According to some further aspects of the present invention, these terms encompass a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, an “expression construct” or “expression cassette” or “construct” or “cassette” refers to a vector suitable for expression of a nucleotide sequence of interest in a plant, such as a recombinant vector. “Expression” refers to the production of a functional product. For example, the expression of a nucleotide sequence may refer to transcription of the nucleotide sequence (such as transcribe to produce an mRNA or a functional RNA) and/or translation of RNA into a protein precursor or a mature protein. “Expression construct” of the invention may be a linear nucleic acid fragment, a circular plasmid, a viral vector, or, in some embodiments, an RNA that can be translated (such as an mRNA. According to further embodiments of the present invention, “expression construct” of the invention may comprise regulatory sequences and nucleotide sequences of interest that are derived from different sources, or regulatory sequences and nucleotide sequences of interest derived from the same source, but arranged in a manner different than that normally found in nature.

The term “regulatory sequence” or “regulatory element” are refer herein to nucleotide sequences located upstream (5′ non-coding sequences), within, or downstream (3′ non-coding sequences) of a coding sequence, and which influence or modulate or control the transcription, RNA processing or stability, or translation of the associated coding sequence. A plant expression regulatory element refers to a nucleotide sequence capable of controlling the transcription, RNA processing or stability or translation of a nucleotide sequence of interest in a plant. Regulatory sequences may include, but are not limited to, promoters, translation leader sequences, terminators, introns, and polyadenylation recognition sequences.

The term “promoter” refers to a nucleic acid fragment capable of controlling transcription of another nucleic acid fragment. In some embodiments of the invention, the promoter is a promoter capable of controlling gene transcription in a plant cell whether or not its origin is from a plant cell. The promoter may be a constitutive promoter or a tissue-specific promoter or a developmentally regulated promoter or an inducible promoter.

“Constitutive promoter” refers to a promoter that generally causes gene expression in most cell types in most circumstances. “Tissue-specific promoter” and “tissue-preferred promoter” are used interchangeably, and refer to a promoter that is expressed predominantly but not necessarily exclusively in one tissue or organ, but that may also be expressed in one specific cell or cell type. “Developmentally regulated promoter” refers to a promoter whose activity is determined by developmental events. “Inducible promoter” selectively expresses a DNA sequence operably linked to it in response to an endogenous or exogenous stimulus (such as environment, hormones, or chemical signals).

As used herein, the term “operably linked” means that a regulatory element (for example but not limited to, a promoter sequence, a transcription termination sequence etc.) is associated to a nucleic acid sequence (such as a coding sequence or an open reading frame), such that the transcription of the nucleotide sequence is controlled and regulated by the transcriptional regulatory element. Techniques for operably linking a regulatory element region to a nucleic acid molecule are known in the art.

The terms “peptide”, “polypeptide”, “protein” and “amino acid sequence” are used interchangeably herein and refer to amino acids in a polymeric form of any length, linked together by peptide bonds. In other words, it encompass a polymer of amino acid residues. The terms apply also to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

According to other aspects of the invention, a “modified” or a “mutant” plant is a plant that has been altered compared to the naturally occurring wild type (WT) plant. Specifically, the endogenous nucleic acid sequences of terpene synthesis gene homologs in Cannabis (nucleic acid sequences encoding CsFPPS1, CsFPPS2, CsGPPS1 and/or CsGPPS2) have been silenced or downregulated or knocked down compared to wild type sequences using gene editing methods as described herein. This causes elimination of expression of endogenous terpene synthesis genes and thus generation of Cannabis plant with significantly less volatile compounds emission, particularly odorless Cannabis or odor free Cannabis.

It should be noted that Cannabis plants of the invention are modified plants compared to wild type plants, which comprise and express mutant alleles, genes or variants of at least one gene encoding CsFPPS1, CsFPPS2, CsGPPS1 and/or CsGPPS2.

It is further noted that a wild type Cannabis plant is a plant that does not have any mutant CsFPPS1, CsFPPS2, CsGPPS1 and/or CsGPPS2-encoding alleles.

In some embodiments of the invention, the guide RNA is a single guide RNA (sgRNA). Methods of constructing suitable sgRNAs according to a given target sequence are known in the art.

It is further within the scope that variants of a particular CsFPPS1, CsFPPS2, CsGPPS1 and/or CsGPPS2 nucleotide or amino acid sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular non-variant CsFPPS1, CsFPPS2, CsGPPS1 and/or CsGPPS2 nucleotide sequence allele as shown in SEQ ID NO 1, 4, 7 and 10; and/or SEQ ID NO 2, 5, 8 and 11; and/or SEQ ID NO 3, 6, 9 and 12 respectively. Sequence alignment programs to determine sequence identity are well known in the art.

Also, the various aspects of the invention encompass not only a CsFPPS1, CsFPPS2, CsGPPS1 and/or CsGPPS2 nucleic acid sequence or amino acid sequence, but also any terpene synthesis gene (e.g. see Table 6) or fragments thereof. By “fragment” is intended a portion of the nucleotide sequence or a portion of the amino acid sequence and hence of the protein encoded thereby. Fragments of a nucleotide sequence may encode protein fragments that retain or not retain the biological activity of the native protein, e.g., enzymatic activity and/or trait.

According to further embodiments of the present invention, DNA introduction into the plant cells can be done by Agrobacterium infiltration, virus based plasmids for delivery of the genome editing molecules and mechanical insertion of DNA (PEG mediated DNA transformation, biolistics, etc.).

In addition, it is within the scope of the present invention that the Cas9 protein is directly inserted together with a gRNA (ribonucleoprotein-RNP's) in order to bypass the need for in vivo transcription and translation of the Cas9+gRNA plasmid in planta to achieve gene editing.

It is also possible to create a genome edited plant and use it as a rootstock. Then, the Cas protein and gRNA can be transported via the vasculature system to the top of the plant and create the genome editing event in the scion part.

It is further within the scope that traits (reduced volatile compounds or odor emission) in Cannabis plants are herein produced by generating gRNA with homology to a specific site or region or domain of predetermined genes in the Cannabis genome i.e. genes encoding CsFPPS1, CsFPPS2, CsGPPS1 and/or CsGPPS2, sub cloning this gRNA into a plasmid containing the Cas9 gene, and insertion of the plasmid into the Cannabis plant cells. In this way insertion, deletion, frameshift or any silencing mutations in at least one of the genes encoding CsFPPS1, CsFPPS2, CsGPPS1 and/or CsGPPS2 are generated thus effectively creating odorless Cannabis plants.

According to one embodiment, the present invention provides a modified Cannabis plant exhibiting reduced volatile organic compounds (VOCs) emission, wherein said modified plant comprises at least one targeted gene modification conferring reduced expression or silencing of at least one gene involved in a terpene biosynthesis pathway.

According to a further embodiment of the present invention, the at least one targeted gene modification confers reduced expression or silencing of at least one gene involved in a terpene biosynthesis pathway as compared to a Cannabis plant lacking said targeted gene modification.

According to a further embodiment of the present invention, the terpene biosynthesis pathway is selected from methylerythritol phosphate (MEP) pathway, mevalonic acid or mevalonate (MEV) pathway, isoprenoid biosynthetic pathway, formation of GPP, FPP and GGPP pathways, formation of squalene pathway, formation of Mono-, Sesqui-und Di-Terpenes pathways, formation of triterpenes from squalene pathway and any combination thereof.

According to a further embodiment of the present invention, the one gene involved in a terpene biosynthesis pathway is selected from CsTPS1PK, CsTPS4PK, CsTPS5PK, CsTPS6PK, CsTPS7PK, CsTPS8PK, CsTPS9PK, CsTPS10PK, CsTPS1 PK, CsTPS12PK, CsTPS13PK, CsTPS14PK, CsTPS15PK, CsTPS16PK, CsTPS17PK, CsTPS18PK, CsTPS19PK, CsTPS20PK, CsTPS21PK, CsTPS22PK, CsTPS23PK, CsTPS24PK, CsTPS25PK, CsTPS26PK, CsTPS27PK, CsTPS30PK, CsTPS31PK, CsTPS32PK, CsTPS33PK, CsTPS34PK, CsTPS35PK, CsTPS12PK, CsTPS13PK, CsTPS1FN, CsTPS2FN, CsTPS3FN, CsTPS4FN, CsTPS5FN, CsTPS6FN, CsTPS7FN, CsTPS8FN, CsTPS9FN, CsTPS11FN, CsDXS1, CsDXS2, CsDXR, CsMCT, CsCMK, CsHDS, CsHDR, CsHMGS, CsHMGR1, CsHMGR2, CsMK, CsPMK, CsMPDC, CsIDI, CsFPPS1, CsFPPS2, CsGPPS1, CsGPPS2 and any combination thereof.

According to a further embodiment of the present invention, the gene involved in a terpene biosynthesis pathway is selected from (a) a gene encoding CsFPPS1 characterized by a sequence selected from SEQ ID NO: 1-3 or a functional variant thereof, (b) a gene encoding CsFPPS2 characterized by a sequence selected from SEQ ID NO: 4-6 or a functional variant thereof, (c) a gene encoding CsGPPS1 characterized by a sequence selected from SEQ ID NO: 7-9 or a functional variant thereof, (d) a gene encoding CsGPPS2 characterized by a sequence selected from SEQ ID NO: 10-12 or a functional variant thereof, and (e) any combination thereof.

According to a further embodiment of the present invention, the functional variant has at least 75% sequence identity to said gene sequence.

According to a further embodiment of the present invention, the gene modification is introduced using mutagenesis, small interfering RNA (siRNA), microRNA (miRNA), artificial miRNA (amiRNA), DNA introgression, endonucleases or any combination thereof.

According to a further embodiment of the present invention, the gene modification is introduced using CRISPR (Clustered Regularly Interspaced Short Palindromic Repeats) and CRISPR-associated (Cas) gene (CRISPR/Cas) system, Transcription activator-like effector nuclease (TALEN), Zinc Finger Nuclease (ZFN), meganuclease or any combination thereof.

According to a further embodiment of the present invention, the targeted gene modification is introduced using (i) at least one RNA-guided endonuclease, or a nucleic acid encoding at least one RNA-guided endonuclease, and (ii) at least one guide RNA (gRNA) or DNA encoding at least one gRNA which directs the endonuclease to a corresponding target sequence within said gene involved in terpene biosynthesis pathway.

According to a further embodiment of the present invention, the targeted gene modification is performed by introducing into a Cannabis plant or a cell thereof a nucleic acid composition comprising: a) a first nucleotide sequence encoding the targeted gRNA molecule and b) a second nucleotide sequence encoding the Cas molecule, or a Cas protein.

According to a further embodiment of the present invention, the gRNA comprises a sequence selected from SEQ ID NO:13-646 and any combination thereof.

According to a further embodiment of the present invention, the gRNA targeted for CsFPPS1, CsFPPS2, CsGPPS1 and/or CsGPPS2 comprises a nucleic acid sequence as set forth in SEQ ID NO: 13-237, SEQ ID NO: 238-390, SEQ ID NO: 391-530 and SEQ ID NO: 531-646, respectively.

According to a further embodiment of the present invention, the gRNA sequence comprises a 3′ Protospacer Adjacent Motif (PAM) selected from the group consisting of NGG (SpCas), NNNNGATT (NmeCas9), NNAGAAW, (StCas9), NAAAAC (TdCas9), NNGRRT (SaCas9) and TBN (Cas-phi).

According to a further embodiment of the present invention, the targeted gene modification is a silencing mutation, a knockdown mutation, a knockout mutation, a loss of function mutation or any combination thereof.

According to a further embodiment of the present invention, the modified plant has reduced odor resulting from volatile compounds emission or is odor free or odorless Cannabis plant.

According to a further embodiment of the present invention, the VOCs are selected from essential oils, secondary metabolites, terpenoids, terpenes, oxygenated and any combination thereof.

According to a further embodiment of the present invention, the VOCs comprise at least one of hemiterpenes, monoterpenes, sesquiterpenes, diterpenes, sesterterpenes, triterpenes, tetraterpenes and polyterpenes.

According to a further embodiment of the present invention, the VOCs are selected from pinene, alpha-pinene, beta-pinene, cis-pinane, trans-pinane, cis-pinanol, trans-pinanol, limonene; linalool; myrcene; eucalyptol; a-phellandrene; b-phellandrene; a-ocimene; b-ocimene, cis-ocimene, ocimene, delta-3-carene; fenchol; sabinene, bomeol, isobomeol, camphene, camphor, phellandrene, a-phellandrene, a-terpinene, geraniol, linalool, nerol, menthol, terpinolene, a-terpinolene, b-terpinolene, g-terpinolene, delta-terpinolene, a-terpineol, trans-2-pinanol, caryophyllene, caryophyllene oxide, humulene, a-humulene, a-bisabolene; b-bisabolene; santalol; selinene; nerolidol, bisabolol; a-cedrene, b-cedrene, b-eudesmol, eudesm-7(11)-en-4-ol, selina-3,7(11)-diene, guaiol, valencene, a-guaiene, beta-guaiene, delta-guaiene, guaiene, famesene, a-famesene, b-famesene, elemene, a-elemene, b-elemene, gamma-elemene, delta-elemene, germacrene, germacrene A, germacrene B, germacrene C, germacrene D, germacrene E, oridonin, phytol, isophytol, ursolic acid, oleanolic acid, and/or 1.5 ene compounds, including guaia-1(10),11-diene, and 1.5 ene. Guaia-1(10), 11-diene.isoprene, α-pinene, β-pinene, d-limonene, β-phellandrene, α-terpinene, α-thujene, γ-terpinene, β-myrcene, (E)-β-ocimene, (−)-limonene, (+)-α-pinene, β-caryophyllene, and α-humulene and any combination thereof.

According to a further embodiment of the present invention, the VOCs in said modified Cannabis plant is measured using gas chromatography-mass spectrometry (GCMS) terpene profiling and quantitation techniques or by any other method for quantifying VOCs.

According to a further embodiment of the present invention, a progeny plant, plant part, plant cell, tissue culture of regenerable cells, protoplasts, callus or plant seed of the modified plant as defined in any of the above are herein provided.

According to a further embodiment, a medical Cannabis product comprising the modified Cannabis plant as defined in any of the above or a part or extract thereof are provided by the present invention.

According to a further embodiment of the present invention, a method for producing a modified Cannabis plant as defined in any of the above is provided. The method comprises introducing using targeted genome modification, at least one genomic modification conferring reduced expression or silencing of at least one gene involved in a terpene biosynthesis pathway.

According to a further embodiment of the present invention, the method as defined in any of the above comprises steps of introducing using genome editing a loss of function mutation in at least one gene involved in a terpene biosynthesis pathway.

According to a further embodiment of the present invention, the method as defined in any of the above comprises steps of: (a) identifying at least one Cannabis gene involved in a terpene biosynthesis pathway; (b) designing and/or synthetizing at least one guide RNA (gRNA) comprising a nucleotide sequence corresponding or complementary to a target sequence is said at least one identified Cannabis gene involved in a terpene biosynthesis pathway; (c) transforming a Cannabis plant cells with endonuclease or nucleic acid encoding endonuclease, together with the at least one gRNA or a DNA encoding the gRNA; (d) optionally, culturing said transformed Cannabis cells; (e) selecting Cannabis plant or plant cells thereof carrying induced targeted loss of function mutation in the at least one gene involved in a terpene biosynthesis pathway; and (f) optionally, regenerating a modified Cannabis plant from said transformed plant cell, plant cell nucleus, or plant tissue.

According to a further embodiment of the present invention, the method as defined in any of the above, comprises silencing or eliminating Cannabis terpene synthesis gene expression comprising steps of: (a) identifying at least one gene locus within a DNA sequence in a Cannabis plant or a cell thereof for CsFPPS1, CsFPPS2, CsGPPS1 and/or CsGPPS2 having a genomic sequence as set for in SEQ ID NO:1, 4, 7 and 10, respectively; (b) identifying at least one custom endonuclease recognition sequence within the at least one locus of CsFPPS1, CsFPPS2, CsGPPS1 and/or CsGPPS2 genes; (c) introducing into the Cannabis plant or a cell thereof at least a first custom gRNA directed endonuclease, wherein the Cannabis plant or a cell thereof comprises the recognition sequence for the custom gRNA directed endonuclease in or proximal to the loci of any one of SEQ ID NO:13-646, and the custom endonuclease is expressed transiently or stably; (d) assaying the Cannabis plant or a cell thereof for a custom endonuclease-mediated modification in the DNA comprising or corresponding to or flanking the loci of any one of SEQ ID NO:13-646; and (e) identifying the Cannabis plant, a cell thereof, or a progeny cell thereof as comprising a modification in the loci of CsFPPS1, CsFPPS2, CsGPPS1 and/or CsGPPS2 genes.

According to a further embodiment of the present invention, wherein the method as defined in any of the above comprises steps of: (a) identifying at least one Cannabis gene involved in a terpene biosynthesis pathway; (b) designing and/or synthetizing at least one guide RNA (gRNA) comprising a nucleotide sequence corresponding or complementary to a target sequence is said at least one identified Cannabis gene involved in a terpene biosynthesis pathway; (c) transforming a Cannabis plant cells with endonuclease or nucleic acid encoding endonuclease, together with the at least one gRNA or a DNA encoding the gRNA; (d) optionally, culturing said transformed Cannabis cells; (e) selecting Cannabis plant or plant cells thereof carrying induced targeted loss of function mutation in the at least one gene involved in a terpene biosynthesis pathway; and (f) optionally, regenerating a modified Cannabis plant from said transformed plant cell, plant cell nucleus, or plant tissue.

In order to understand the invention and to see how it may be implemented in practice, a plurality of preferred embodiments will now be described, by way of non-limiting example only, with reference to the following examples.

### Example 1

A process for generating genome edited Cannabis plants

This example describes a generalized scheme of the process for generating the genome edited Cannabis plants of the present invention. The process comprises the following steps:
1. Designing and synthesizing gRNA's corresponding to a sequence targeted for editing. Editing event should be designed flanking with a unique restriction site sequence to allow easier screening of successful editing.
2. Carrying transformation using Agrobacterium or biolistics. For Agrobacterium and bioloistics transformation using a DNA plasmid, a vector containing a selection marker, Cas9 gene and relevant gRNA's is constructed. For biolistics using Ribonucleoprotein (RNP) complexes, RNP complexes are created by mixing the Cas9 protein with relevant gRNA's.
3. Performing regeneration in tissue culture. For DNA transformation, using antibiotics for selection of positive transformants.
4. Selecting positive transformants. Once regenerated plants appear in the regenerated tissue culture, obtaining leaf (or any other selected tissue) samples, extracting DNA from the obtained sample and preforming PCR using primers flanking the editing region. The resulted PCR products are digested with enzymes recognizing the restriction site near original gRNA sequence. If editing event occurred, the restriction site will be disrupted and PCR product will not be cleaved. Absence of an editing event will result in a cleaved PCR product.

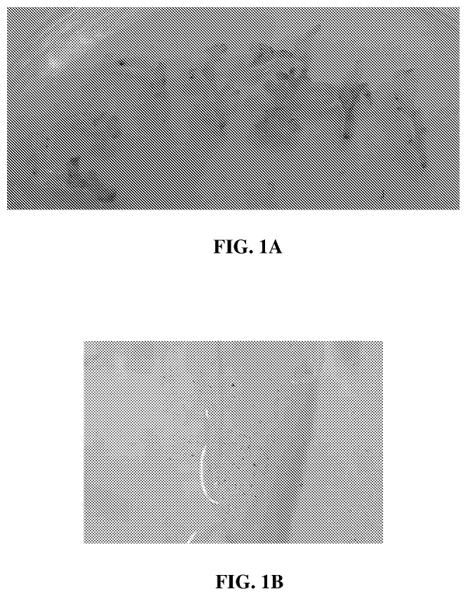

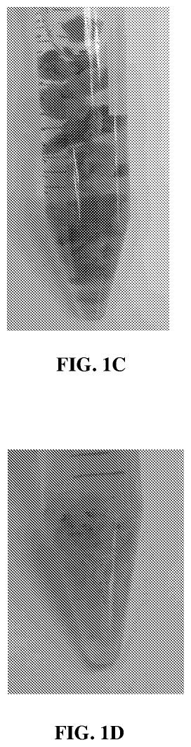

Reference is now made to FIG. 1A-D photographically presenting GUS staining of Cannabis tissues transformed with GUS reporter gene. In this figure the following transformed Cannabis tissues are shown: axillary buds (FIG. 1A), mature leaf (FIG. 1B), calli (FIG. 1C), and cotyledons (FIG. 1D). FIG. 1 demonstrates that various Cannabis tissues have been successfully transformed (e.g. using biolistics system). Transformation has been performed into calli, leaves, axillary buds and cotyledons of Cannabis.

According to some embodiments of the present invention, transformation of various Cannabis tissues was performed using particle bombardment of:
DNA vectors
Ribonucleoprotein complex (RNP's)

According to further embodiments of the present invention, transformation of various Cannabis tissues was performed using Agrobacterium (Agrobacterium tumefaciens) by:
Regeneration-based transformation
Floral-dip transformation
Seedling transformation

Transformation efficiency by A. tumefaciens has been compared to the bombardment method by transient GUS transformation experiment. After transformation, GUS staining of the transformants has been performed.

According to further embodiments of the present invention, additional transformation tools were used in Cannabis, including, but not limited to:
Protoplast PEG transformation
Extend RNP use
Directed editing screening using fluorescent tags
Electroporation

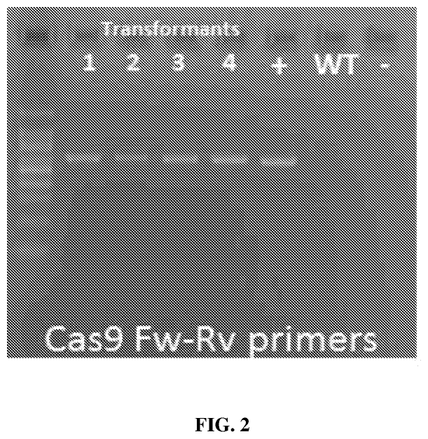

Selection of positive transformants is performed on DNA extracted from leaf sample of regenerated transformed plants and PCR is performed using primers flanking the edited region. PCR products are then digested with enzymes recognizing the restriction site near the original gRNA sequence. If editing event occurred, the restriction site will be disrupted and the PCR product will not be cleaved. No editing event will result in a cleaved PCR product. Reference is now made to FIG. 2 showing PCR detection of Cas9 DNA in transformed Cannabis plants. The figure illustrates PCR detection of transformed leaf tissue screened for the presence of the Cas9 gene two weeks post transformation. The PCR products of the Cas9 gene were amplified from four transformed plants two weeks post transformation. This figure shows that two weeks post transformation, Cas9 DNA was detected in shoots of transformed Cannabis plants.

Screening for CRISPR/Cas9 gene editing events has been performed by at least one of the following analysis methods:
Restriction Fragment Length Polymorphism (RFLP)
Next Generation Sequencing (NGS)
PCR fragment analysis
Fluorescent-tag based screening
High resolution melting curve analysis (HRMA)

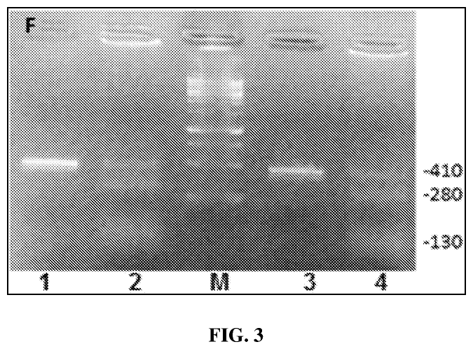

Reference is now made to FIG. 3 illustrating in vivo specific DNA cleavage by Cas9+gRNA (RNP) complex, as an embodiment of the present invention. This figure presents results of analysis of CRISPR/Cas9 cleavage activity on samples 1 and 2 shown in FIG. 2, where (1) Sample 1 PCR product (no DNA digest); (2) Sample 1 PCR product+RNP (digested DNA); (3) Sample 2 PCR product (no DNA digest); (4) Sample 2 PCR product+RNP (digested DNA); (M) marker.

FIG. 3 shows successful digestion of the resulted PCR amplicon containing the gene specific gRNA sequence, by RNP complex containing Cas9. The analysis included the following steps:
1) Amplicon was isolated from two exemplified Cannabis strains by primers flanking the sequence of the gene of interest targeted by the predesigned gRNA.
2) RNP complex was incubated with the isolated amplicon.
3) The reaction mix was then loaded on agarose gel to evaluate Cas9 cleavage activity at the target site.

Selection of odorless transformed Cannabis plants was performed.

It is within the scope that different gRNA promoters were tested in order to maximize editing efficiency.

It is noted that line stabilization may be performed by the following:
Induction of male flowering on female (XX) plants
Self pollination

According to some embodiments of the present invention, line stabilization requires about 6 self-crossing (6 generations) and done through a single seed descent (SSD) approach.

F1 hybrid seed production: Novel hybrids are produced by crosses between different Cannabis strains.

According to a further aspect of the current invention, shortening line stabilization is performed by Doubled Haploids (DH). More specifically, the CRISPR-Cas9 (or CRISPR-nCas9) system is transformed into microspores to achieve DH homozygous parental lines. A doubled haploid (DH) is a genotype formed when haploid cells undergo chromosome doubling. Artificial production of doubled haploids is important in plant breeding. It is herein acknowledged that conventional inbreeding procedures take about six generations to achieve approximately complete homozygosity, whereas doubled haploidy achieves it in one generation.

It is within the scope of the current invention that genetic markers specific for Cannabis are developed and provided by the current invention:
Sex markers—molecular markers are used for identification and selection of female vs male plants in the herein disclosed breeding program
Genotyping markers—germplasm used in the current invention is genotyped using molecular markers, in order to allow a more efficient breeding process and identification of the HR-related genes one or more editing events.

It is further within the scope of the current invention that allele and genetic variation is analyzed for the Cannabis strains used.

### Example 2

Targeting Genes Involved in Terpene Synthesis in Cannabis

At the aim of producing odorless Cannabis plant, Cannabis sativa (C. sativa) genes encoding terpene synthesis proteins were identified. The homologous terpene synthesis alleles found have been sequenced and mapped.

Cannabis FPPS1 (CsFPPS1) encodes a Farnesyl diphosphate synthase protein. The CsFPPS1 gene locus was mapped to CM010796.2:5549971-5554777 and has a genomic sequence as set forth in SEQ ID NO:1. The CsFPPS1 gene has a coding sequence (CDS) as set forth in SEQ ID NO:2 and it encodes an amino acid sequence as set forth in SEQ ID NO:3.

Cannabis FPPS2 (CsFPPS2) encodes a Farnesyl diphosphate synthase protein. The CsFPPS2 gene locus was mapped to CM010792.2: 72694075-72697000 and has a genomic sequence as set forth in SEQ ID NO:4. The CsFPPS2 gene has a coding sequence (CDS) as set forth in SEQ ID NO:5 and it encodes an amino acid sequence as set forth in SEQ ID NO:6.

Cannabis GPPS1 (CsGPPS1) encodes a Geranyl diphosphate synthase protein. The CsGPPS1 gene locus was mapped to CM010792.2: 55682615-55684286 and has a genomic sequence as set forth in SEQ ID NO:7. The CsGPPS1 gene has a coding sequence (CDS) as set forth in SEQ ID NO:8 and it encodes an amino acid sequence as set forth in SEQ ID NO:9.

Cannabis GPPS2 (CsGPPS2) encodes a Geranyl diphosphate synthase protein. The CsGPPS2 gene locus was mapped to CsGPPS.ssu2 CM010795.2: 1123757-1125219 and has a genomic sequence as set forth in SEQ ID NO:10. The CsGPPS2 gene has a coding sequence (CDS) as set forth in SEQ ID NO:11 and it encodes an amino acid sequence as set forth in SEQ ID NO:12.

At the next stage, gRNA molecules corresponding to the sequence targeted for editing were designed and synthesized, i.e. sequences targeted each of the genes CsFPPS1, CsFPPS2, CsGPPS1 and CsGPPS2. It is noted that the editing event is preferably targeted to a unique restriction site sequence to allow easier screening for plants carrying an editing event within their genome. According to some aspects of the invention, the nucleotide sequence of the gRNAs should be completely compatible with the genomic sequence of the target gene. Therefore, for example, suitable gRNA molecules should be constructed for different GPPS or FPPS homologues/alleles of different Cannabis strains.

The designed gRNA molecules were cloned into suitable vectors and their sequence has been verified. In addition, different Cas9 versions have been analyzed for optimal compatibility between the Cas9 protein activity and the gRNA molecule in the Cannabis plant.

Reference is now made to Tables 1, 2, 3 and 4 presenting gRNA sequences constructed for silencing CsFPPS1, CsFPPS2, CsGPPS1 and CsGPPS2 genes, respectively. In Tables 1, 2, 3 and 4 the term ‘PAM’ refers to protospacer adjacent motif, which is a 2-6 base pair DNA sequence immediately following the DNA sequence targeted by the Cas9 nuclease in the CRISPR bacterial adaptive immune system. The genomic DNA sense strand is marked as “1”, and the antisense strand is marked as “−1”.

TABLE 1
gRNA and complementing PAM sequences of CsFPPS1

| Position | | | | SEQ |
|---|---|---|---|---|
| in SEQ | | | | ID |
| ID NO: 1 | Strand | Sequence | PAM | NO |
| 286 | 1 | AATAGAATAATCTTCACAGA | TGG | 13 |
| 287 | 1 | ATAGAATAATCTTCACAGAT | GGG | 14 |
| 301 | -1 | AAAAGTTTGGCATTTTCATC | TGG | 15 |
| 314 | -1 | CTTAACCACGAAGAAAAGTT | TGG | 16 |
| 320 | 1 | AAATGCCAAACTTTTCTTCG | TGG | 17 |
| 340 | 1 | TGGTTAAGTGTTAACTATAA | TGG | 18 |
| 361 | 1 | GGTAATGTTTGTAATTAACG | CGG | 19 |
| 368 | 1 | TTTGTAATTAACGCGGAAAG | TGG | 20 |
| 381 | -1 | CTCGATTTTCATTCGTAAAT | GGG | 21 |
| 382 | -1 | ACTCGATTTTCATTCGTAAA | TGG | 22 |
| 420 | -1 | ATATGAGAGGGAACGAAGTG | AGG | 23 |
| 432 | -1 | CCGAGTGTGCTTATATGAGA | GGG | 24 |
| 433 | -1 | ACCGAGTGTGCTTATATGAG | AGG | 25 |
| 443 | 1 | CCCTCTCATATAAGCACACT | CGG | 26 |
| 494 | 1 | AGCTCTATCACTCGCTTCCA | TGG | 27 |
| 497 | 1 | TCTATCACTCGCTTCCATGG | CGG | 28 |
| 500 | -1 | CTTGGCCTTTAGATCCGCCA | TGG | 29 |
| 506 | 1 | CGCTTCCATGGCGGATCTAA | AGG | 30 |
| 518 | -1 | GGAGTAGACATTCAAGAACT | TGG | 31 |
| 539 | -1 | AAGGAGCTCTGATTTCAAAA | CGG | 32 |
| 558 | -1 | ATTCGAAAGCTGGATCTTGA | AGG | 33 |
| 568 | -1 | ATATCAGTGAATTCGAAAGC | TGG | 34 |
| 592 | 1 | TCACTGATATTTCTCGTCAA | TGG | 35 |
| 593 | 1 | CACTGATATTTCTCGTCAAT | GGG | 36 |
| 596 | 1 | TGATATTTCTCGTCAATGGG | TGG | 37 |
| 601 | 1 | TTTCTCGTCAATGGGTGGAG | CGG | 38 |
| 602 | 1 | TTCTCGTCAATGGGTGGAGC | GGG | 39 |
| 701 | 1 | TTTTCTTTCTTATCATAATG | AGG | 40 |
| 706 | 1 | TTTCTTATCATAATGAGGTA | CGG | 41 |
| 735 | 1 | TTTTACGTTATAATTAGTAG | TGG | 42 |
| 740 | 1 | CGTTATAATTAGTAGTGGAG | TGG | 43 |
| 756 | 1 | GGAGTGGATTGAGTTATAAT | TGG | 44 |
| 1926 | 1 | AATTATCAAAGTACAACTCA | AGG | 45 |
| 1927 | 1 | ATTATCAAAGTACAACTCAA | GGG | 46 |
| 1958 | 1 | ATGTATTTATTGTTACATTA | TGG | 47 |
| 1980 | 1 | GCTAATTTCAATGTATATGT | TGG | 48 |
| 2041 | -1 | AACACAATTAGGAAACTACA | AGG | 49 |
| 2052 | -1 | CCAAAATATACAACACAATT | AGG | 50 |
| 2063 | 1 | CCTAATTGTGTTGTATATTT | TGG | 51 |
| 2092 | 1 | ATGACAGACTACAATGTTCC | TGG | 52 |
| 2095 | 1 | ACAGACTACAATGTTCCTGG | AGG | 53 |
| 2099 | 1 | ACTACAATGTTCCTGGAGGT | TGG | 54 |
| 2100 | 1 | CTACAATGTTCCTGGAGGTT | GGG | 55 |
| 2132 | 1 | TTTTTATAATTAAATTGTTG | AGG | 56 |
| 2159 | 1 | AATAAAGAGTTCTCCAAAAG | AGG | 57 |
| 2160 | 1 | ATAAAGAGTTCTCCAAAAGA | GGG | 58 |
| 2161 | -1 | GAGTCATTTTCACCCTCTTT | TGG | 59 |
| 2210 | 1 | AACTGCTTCTGATGCAGCTC | TGG | 60 |
| 2211 | 1 | ACTGCTTCTGATGCAGCTCT | GGG | 61 |
| 2264 | 1 | GTCTTTACTGATGCATCTCT | TGG | 62 |
| 2265 | 1 | TCTTTACTGATGCATCTCTT | GGG | 63 |
| 2284 | 1 | TGGGTGATATTTTATGTTGC | AGG | 64 |
| 2285 | 1 | GGGTGATATTTTATGTTGCA | GGG | 65 |
| 2299 | 1 | GTTGCAGGGAAATTAAACCG | AGG | 66 |
| 2305 | -1 | TGTCGATAACTGATAGGCCT | CGG | 67 |
| 2311 | -1 | TGTAGCTGTCGATAACTGAT | AGG | 68 |
| 2335 | 1 | GACAGCTACAAGCTGTTGAA | AGG | 69 |
| 2355 | 1 | AGGAGAAGAGTTGACTGAAG | AGG | 70 |
| 2379 | -1 | AATGCACCAACCAAGAGCAC | TGG | 71 |
| 2380 | 1 | ATCTTTCTAGCCAGTGCTCT | TGG | 72 |
| 2384 | 1 | TTCTAGCCAGTGCTCTTGGT | TGG | 73 |
| 2396 | 1 | CTCTTGGTTGGTGCATTGAA | TGG | 74 |
| 2397 | 1 | TCTTGGTTGGTGCATTGAAT | GGG | 75 |
| 2426 | -1 | AAGATAGCCAAGGAGGAGAG | TGG | 76 |
| 2430 | 1 | TTAATTACCACTCTCCTCCT | TGG | 77 |
| 2433 | -1 | ACCAACCAAGATAGCCAAGG | AGG | 78 |
| 2436 | -1 | TCCACCAACCAAGATAGCCA | AGG | 79 |
| 2439 | 1 | ACTCTCCTCCTTGGCTATCT | TGG | 80 |
| 2443 | 1 | TCCTCCTTGGCTATCTTGGT | TGG | 81 |
| 2446 | 1 | TCCTTGGCTATCTTGGTTGG | TGG | 82 |
| 2453 | 1 | CTATCTTGGTTGGTGGAGCC | TGG | 83 |
| 2460 | -1 | TCTCTCATTCATAAAATTCC | AGG | 84 |
| 2536 | 1 | GCAGCTGCAAGCATACTTTC | TGG | 85 |
| 2554 | 1 | TCTGGTTCTTGATGACATTA | TGG | 86 |
| 2571 | 1 | TTATGGACAACTCACACACG | CGG | 87 |
| 2576 | 1 | GACAACTCACACACGCGGCG | TGG | 88 |
| 2588 | -1 | GAACTTTATACCAGCAAGGC | TGG | 89 |
| 2589 | 1 | CGCGGCGTGGCCAGCCTTGC | TGG | 90 |
| 2592 | -1 | TTGGGAACTTTATACCAGCA | AGG | 91 |
| 2605 | 1 | TTGCTGGTATAAAGTTCCCA | AGG | 92 |
| 2610 | -1 | TTCAATGAGGTACAAACCTT | GGG | 93 |
| 2611 | -1 | ATTCAATGAGGTACAAACCT | TGG | 94 |
| 2623 | -1 | GAGATTATACTTATTCAATG | AGG | 95 |
| 2670 | 1 | ATAAAATCGCTGTTTTCATG | TGG | 96 |
| 2706 | 1 | TATGTGAACTTTTATCATCA | AGG | 97 |
| 2710 | 1 | TGAACTTTTATCATCAAGGT | TGG | 98 |
| 2731 | 1 | GGAATGATTGCAGCAAATGA | TGG | 99 |
| 2732 | 1 | GAATGATTGCAGCAAATGAT | GGG | 100 |
| 2733 | 1 | AATGATTGCAGCAAATGATG | GGG | 101 |
| 2758 | -1 | TCTTAAGAATTCTGAAAATA | TGG | 102 |
| 2781 | 1 | AATTCTTAAGAATCACTTCA | AGG | 103 |
| 2798 | -1 | TCAAGCAGATCAACGTAGTA | TGG | 104 |
| 2823 | 1 | TCTGCTTGATTTGTTCAATG | AGG | 105 |
| 2857 | -1 | GGGGGGGGGGGGGTGGAACT | AGG | 106 |
| 2871 | -1 | AAGAAGGGGGGGGGGGGGGG | GGG | 107 |
| 2872 | -1 | GAAGAAGGGGGGGGGGGGGG | GGG | 108 |
| 2873 | -1 | AGAAGAAGGGGGGGGGGGGG | GGG | 109 |
| 2874 | -1 | AAGAAGAAGGGGGGGGGGGG | GGG | 110 |
| 2875 | -1 | GAAGAAGAAGGGGGGGGGGG | GGG | 111 |
| 2876 | -1 | AGAAGAAGAAGGGGGGGGGG | GGG | 112 |
| 2877 | -1 | GAGAAGAAGAAGGGGGGGGG | GGG | 113 |
| 2878 | -1 | AGAGAAGAAGAAGGGGGGGG | GGG | 114 |
| 2879 | -1 | GAGAGAAGAAGAAGGGGGGG | GGG | 115 |
| 2880 | -1 | AGAGAGAAGAAGAAGGGGGG | GGG | 116 |
| 2881 | -1 | GAGAGAGAAGAAGAAGGGGG | GGG | 117 |
| 2882 | -1 | AGAGAGAGAAGAAGAAGGGG | GGG | 118 |
| 2883 | -1 | GAGAGAGAGAAGAAGAAGGG | GGG | 119 |
| 2884 | -1 | AGAGAGAGAGAAGAAGAAGG | GGG | 120 |
| 2885 | -1 | GAGAGAGAGAGAAGAAGAAG | GGG | 121 |
| 2886 | -1 | AGAGAGAGAGAGAAGAAGAA | GGG | 122 |
| 2887 | -1 | GAGAGAGAGAGAGAAGAAGA | AGG | 123 |
| 2933 | -1 | TGGAACTCCACCTATACAAG | AGG | 124 |
| 2934 | 1 | CGAATAAATACCTCTTGTAT | AGG | 125 |
| 2937 | 1 | ATAAATACCTCTTGTATAGG | TGG | 126 |
| 2953 | -1 | GCATTTGTCCTGAAGCGGTT | TGG | 127 |
| 2956 | 1 | GTGGAGTTCCAAACCGCTTC | AGG | 128 |
| 2958 | -1 | GTCTAGCATTTGTCCTGAAG | CGG | 129 |
| 2986 | 1 | CTAGACTTAATTTCGAGTGA | AGG | 130 |
| 2987 | 1 | TAGACTTAATTTCGAGTGAA | GGG | 131 |
| 2988 | 1 | AGACTTAATTTCGAGTGAAG | GGG | 132 |
| 3073 | 1 | ATTAAATAGTGACTAAATTA | AGG | 133 |
| 3083 | 1 | GACTAAATTAAGGATCCTTT | TGG | 134 |
| 3087 | -1 | CATTTTTATGAAAAACCAAA | AGG | 135 |
| 3116 | -1 | ATATAATGCCAACATTTTCA | TGG | 136 |
| 3119 | 1 | TGAGCAATCCATGAAAATGT | TGG | 137 |
| 3144 | -1 | TTCCTCCAAACTTACGTATT | TGG | 138 |
| 3150 | 1 | TGCAGCCAAATACGTAAGTT | TGG | 139 |
| 3153 | 1 | AGCCAAATACGTAAGTTTGG | AGG | 140 |
| 3214 | 1 | CGCACTTTACTCGATTATAA | AGG | 141 |
| 3245 | 1 | GTTGTATAAATAGAGAGACA | TGG | 142 |
| 3246 | 1 | TTGTATAAATAGAGAGACAT | GGG | 143 |
| 3279 | -1 | TTATGGAGTATAATGCAAAA | CGG | 144 |
| 3296 | -1 | GGACATTGAACAGAGTATTA | TGG | 145 |
| 3317 | -1 | GCAAACACTTGAAATTACAA | GGG | 146 |
| 3318 | -1 | AGCAAACACTTGAAATTACA | AGG | 147 |
| 3347 | 1 | TTGCTAATATTACATTTGTT | TGG | 148 |
| 3373 | -1 | TTTTGTACTGAACAATGCGG | CGG | 149 |
| 3376 | -1 | CAGTTTTGTACTGAACAATG | CGG | 150 |
| 3399 | -1 | TGAAAGGTAAAATGAATAAT | AGG | 151 |
| 3415 | -1 | ATAAAATAATACTCACTGAA | AGG | 152 |
| 3438 | -1 | CATCGGATGCTTTTACTTGC | TGG | 153 |
| 3455 | -1 | GTTTATGGAAAAAAAGTCAT | CGG | 154 |
| 3470 | -1 | GGACAGATATTGAATGTTTA | TGG | 155 |
| 3491 | -1 | AGTGCAAATAAGGGGCGAAA | TGG | 156 |
| 3499 | -1 | GCACAAGGAGTGCAAATAAG | GGG | 157 |
| 3500 | -1 | GGCACAAGGAGTGCAAATAA | GGG | 158 |
| 3501 | -1 | TGGCACAAGGAGTGCAAATA | AGG | 159 |
| 3514 | -1 | AGTACATTTGGGGTGGCACA | AGG | 160 |
| 3521 | -1 | AGATGCAAGTACATTTGGGG | TGG | 161 |
| 3524 | -1 | TCTAGATGCAAGTACATTTG | GGG | 162 |
| 3525 | -1 | TTCTAGATGCAAGTACATTT | GGG | 163 |
| 3526 | -1 | ATTCTAGATGCAAGTACATT | TGG | 164 |
| 3556 | 1 | GAATCTTGTTACAAGATTTT | TGG | 165 |
| 3557 | 1 | AATCTTGTTACAAGATTTTT | GGG | 166 |
| 3570 | -1 | TTTTCACAGGCATTTCAAGA | AGG | 167 |
| 3583 | -1 | GCAATGACTCTGATTTTCAC | AGG | 168 |
| 3616 | -1 | CATGCAACCTGTGTAGATAT | GGG | 169 |
| 3617 | -1 | ACATGCAACCTGTGTAGATA | TGG | 170 |
| 3620 | 1 | TGCATTTCCCATATCTACAC | AGG | 171 |
| 3645 | 1 | GCATGTGCATTGCTTATGTC | AGG | 172 |
| 3646 | 1 | CATGTGCATTGCTTATGTCA | GGG | 173 |
| 3647 | 1 | ATGTGCATTGCTTATGTCAG | GGG | 174 |
| 3672 | -1 | GAATGTTCTTGACATCAACA | TGG | 175 |
| 3695 | 1 | CAAGAACATTCTTGTTCAGA | TGG | 176 |
| 3696 | 1 | AAGAACATTCTTGTTCAGAT | GGG | 177 |
| 3716 | 1 | GGGAATCTACTTTCAAGTAC | AGG | 178 |
| 3737 | 1 | GGTAAGTTTTCTGTTAAGCA | TGG | 179 |
| 3793 | 1 | TAAAGCATTTATGAAACATC | TGG | 180 |
| 3859 | 1 | CGAGTGTTTATGTTGTGTAC | TGG | 181 |
| 3892 | -1 | GTCGTCCTATTAGAAAGAGA | AGG | 182 |
| 3898 | 1 | ATCTGCCTTCTCTTTCTAAT | AGG | 183 |
| 3910 | 1 | TTTCTAATAGGACGACTATT | TGG | 184 |
| 3936 | -1 | CTTACCTTACCAAGGATCTT | AGG | 185 |
| 3938 | 1 | TTTGTTGATCCTAAGATCCT | TGG | 186 |
| 3943 | 1 | TGATCCTAAGATCCTTGGTA | AGG | 187 |
| 3944 | -1 | TTAGCTTGCTTACCTTACCA | AGG | 188 |
| 3981 | -1 | AGACTTATTTCGGTTACTGG | TGG | 189 |
| 3984 | -1 | AATAGACTTATTTCGGTTAC | TGG | 190 |
| 3991 | -1 | TAAATGTAATAGACTTATTT | CGG | 191 |
| 4018 | 1 | ACATTTACATTTTTGTTTAA | TGG | 192 |
| 4033 | -1 | AGGAGAAAGGACCTATATTA | GGG | 193 |
| 4034 | -1 | TAGGAGAAAGGACCTATATT | AGG | 194 |
| 4046 | -1 | GTTCCTATCTGATAGGAGAA | AGG | 195 |
| 4053 | -1 | AATGTCTGTTCCTATCTGAT | AGG | 196 |
| 4054 | 1 | GGTCCTTTCTCCTATCAGAT | AGG | 197 |
| 4085 | 1 | TTGAAGATTTCAAGTGTTCT | TGG | 198 |
| 4089 | 1 | AGATTTCAAGTGTTCTTGGT | TGG | 199 |
| 4104 | 1 | TTGGTTGGTTGTTAAAGCAT | TGG | 200 |
| 4119 | 1 | AGCATTGGAGCTCAGCAATG | AGG | 201 |
| 4149 | 1 | GAAAATATTAAATGTGAGAC | TGG | 202 |
| 4187 | -1 | AAGCAAACTGATTTTTGATA | AGG | 203 |
| 4219 | 1 | TTACTTTTGATGTTTGTTCC | AGG | 204 |
| 4226 | -1 | CTGCCTTGCCATAGTTCTCC | TGG | 205 |
| 4229 | 1 | TGTTTGTTCCAGGAGAACTA | TGG | 206 |
| 4234 | 1 | GTTCCAGGAGAACTATGGCA | AGG | 207 |
| 4243 | 1 | GAACTATGGCAAGGCAGACC | CGG | 208 |
| 4250 | -1 | TTACTTTAGCTACTTTTTCC | GGG | 209 |
| 4251 | -1 | TTTACTTTAGCTACTTTTTC | CGG | 210 |
| 4276 | 1 | TAAAGTAAAAGCCCTCTACA | AGG | 211 |
| 4277 | -1 | CAAGATCAAGCTCCTTGTAG | AGG | 212 |
| 4291 | 1 | CTACAAGGAGCTTGATCTTG | AGG | 213 |
| 4307 | -1 | AAGAAGGTTTCAGAGTTTGA | TGG | 214 |
| 4323 | -1 | TTATTAAGTTTTATATAAGA | AGG | 215 |
| 4364 | -1 | CTAATATATATGTATGCAGA | TGG | 216 |
| 4394 | -1 | AAATTCACCCTGCAAAGTAC | GGG | 217 |
| 4395 | -1 | CAAATTCACCCTGCAAAGTA | CGG | 218 |
| 4397 | 1 | GTATATAACCCGTACTTTGC | AGG | 219 |
| 4398 | 1 | TATATAACCCGTACTTTGCA | GGG | 220 |
| 4470 | -1 | CTGCTTGCACAGCTTTGCTG | GGG | 221 |
| 4471 | -1 | ACTGCTTGCACAGCTTTGCT | GGG | 222 |
| 4472 | -1 | CACTGCTTGCACAGCTTTGC | TGG | 223 |
| 4499 | 1 | AGCAGTGTTGAAGTCTTTCT | TGG | 224 |
| 4500 | 1 | GCAGTGTTGAAGTCTTTCTT | GGG | 225 |
| 4516 | 1 | TCTTGGGTAAGATATACAAA | AGG | 226 |
| 4551 | 1 | AGTTATCAAATTCCAAGAAC | AGG | 227 |
| 4552 | 1 | GTTATCAAATTCCAAGAACA | GGG | 228 |
| 4555 | 1 | ATCAAATTCCAAGAACAGGG | AGG | 229 |
| 4559 | 1 | AATTCCAAGAACAGGGAGGA | AGG | 230 |
| 4563 | 1 | CCAAGAACAGGGAGGAAGGA | AGG | 231 |
| 4567 | 1 | GAACAGGGAGGAAGGAAGGA | AGG | 232 |
| 4572 | 1 | GGGAGGAAGGAAGGAAGGAA | AGG | 233 |
| 2099 | -1 | ATTGCACAATCCCAACCTCC | AGG | 234 |
| 4033 | 1 | TTTAATGGAGTCCCTAATAT | AGG | 235 |
| 4276 | -1 | AAGATCAAGCTCCTTGTAGA | GGG | 236 |
| 4552 | -1 | CCTTCCTTCCTCCCTGTTCT | TGG | 237 |

TABLE 2
gRNA and complementing PAM sequences of CsFPPS2

| Position | | | | SEQ |
|---|---|---|---|---|
| in SEQ | | | | ID |
| ID NO: 4 | Strand | Sequence | PAM | NO |
| 113 | 1 | TTTATATAATTTGTTTGAAA | TGG | 238 |
| 177 | 1 | GATTTTAAACATTATTTAAT | TGG | 239 |
| 190 | 1 | ATTTAATTGGTCAATACAAG | TGG | 240 |
| 202 | -1 | CATAGACCACTGGAGTTTGG | AGG | 241 |
| 205 | -1 | GTTCATAGACCACTGGAGTT | TGG | 242 |
| 207 | 1 | AAGTGGCCTCCAAACTCCAG | TGG | 243 |
| 212 | -1 | GTACTCTGTTCATAGACCAC | TGG | 244 |
| 236 | -1 | GAGAGAGAGAGAGTCAGTGT | AGG | 245 |
| 315 | 1 | ATATAGATTTTCAGTATCAC | AGG | 246 |
| 316 | 1 | TATAGATTTTCAGTATCACA | GGG | 247 |
| 342 | -1 | AACAAAGGTAGGACTCGAAT | GGG | 248 |
| 343 | -1 | CAACAAAGGTAGGACTCGAA | TGG | 249 |
| 353 | -1 | AACACAAACACAACAAAGGT | AGG | 250 |
| 357 | -1 | AACAAACACAAACACAACAA | AGG | 251 |
| 395 | -1 | ATCACTCATTTTTATTTTTT | TGG | 252 |
| 425 | 1 | TGATTTAAAGTCCAAATTCA | TGG | 253 |
| 425 | -1 | GTAGTAAACCTCCATGAATT | TGG | 254 |
| 428 | 1 | TTTAAAGTCCAAATTCATGG | AGG | 255 |
| 474 | -1 | CATCGGTAAACTCGAAAGCA | GGG | 256 |
| 475 | -1 | TCATCGGTAAACTCGAAAGC | AGG | 257 |
| 491 | -1 | GACCCATTGGCGAGAATCAT | CGG | 258 |
| 499 | 1 | TTACCGATGATTCTCGCCAA | TGG | 259 |
| 500 | 1 | TACCGATGATTCTCGCCAAT | GGG | 260 |
| 504 | -1 | AGAATACCTGTTCGACCCAT | TGG | 261 |
| 509 | 1 | TTCTCGCCAATGGGTCGAAC | AGG | 262 |
| 528 | -1 | ATGGAGAGAGTTAGAGAAAT | TGG | 263 |
| 547 | -1 | TTCCATAAAATGAAAAACAA | TGG | 264 |
| 556 | 1 | CTCCATTGTTTTTCATTTTA | TGG | 265 |
| 563 | 1 | GTTTTTCATTTTATGGAATT | TGG | 266 |
| 564 | 1 | TTTTTCATTTTATGGAATTT | GGG | 267 |
| 565 | 1 | TTTTCATTTTATGGAATTTG | GGG | 268 |
| 583 | -1 | GACTTAACAAAAAAAAAAAA | AGG | 269 |
| 610 | -1 | AAAAGGACTAAAAACGAATC | TGG | 270 |
| 627 | -1 | AACAAAATCATGAATTAAAA | AGG | 271 |
| 683 | 1 | CTTTTAGCTTAATGATTTAG | TGG | 272 |
| 684 | 1 | TTTTAGCTTAATGATTTAGT | GGG | 273 |
| 825 | 1 | ATTTTGACTTTTGCAGATGT | TGG | 274 |
| 841 | 1 | ATGTTGGATTACAATGTCCC | AGG | 275 |
| 844 | 1 | TTGGATTACAATGTCCCAGG | AGG | 276 |
| 847 | -1 | ATTCTCAAAACAAACCTCCT | GGG | 277 |
| 848 | -1 | CATTCTCAAAACAAACCTCC | TGG | 278 |
| 885 | -1 | ATAAGAAATTTGTTTAAACA | AGG | 279 |
| 925 | 1 | TGATTTTCTTTGTTCTTGTT | TGG | 280 |
| 929 | 1 | TTTCTTTGTTCTTGTTTGGT | AGG | 281 |
| 944 | 1 | TTGGTAGGTAAACTTAATAG | AGG | 282 |
| 945 | 1 | TGGTAGGTAAACTTAATAGA | GGG | 283 |
| 977 | -1 | CCTTTCCTCCTTTAAGAATT | TGG | 284 |
| 980 | 1 | GATAGTTACCAAATTCTTAA | AGG | 285 |
| 983 | 1 | AGTTACCAAATTCTTAAAGG | AGG | 286 |
| 988 | 1 | CCAAATTCTTAAAGGAGGAA | AGG | 287 |
| 1028 | 1 | ATTTTCTTAACTTCTGCTCT | TGG | 288 |
| 1032 | 1 | TCTTAACTTCTGCTCTTGGT | TGG | 289 |
| 1044 | 1 | CTCTTGGTTGGTGTATTGAA | TGG | 290 |
| 1045 | 1 | TCTTGGTTGGTGTATTGAAT | GGG | 291 |
| 1063 | 1 | ATGGGTATGCAACTCATTTT | TGG | 292 |
| 1064 | 1 | TGGGTATGCAACTCATTTTT | GGG | 293 |
| 1067 | 1 | GTATGCAACTCATTTTTGGG | AGG | 294 |
| 1092 | 1 | AATTTTTTCAATTCATCAAT | TGG | 295 |
| 1093 | 1 | ATTTTTTCAATTCATCAATT | GGG | 296 |
| 1179 | 1 | TCTTGTTCTTGATGATATCA | TGG | 297 |
| 1188 | 1 | TGATGATATCATGGATAACT | CGG | 298 |
| 1201 | 1 | GATAACTCGGTTACACGTCG | CGG | 299 |
| 1214 | 1 | CACGTCGCGGTCAACCTTGC | TGG | 300 |
| 1217 | -1 | TTTGGTACTCTAAACCAGCA | AGG | 301 |
| 1230 | 1 | TTGCTGGTTTAGAGTACCAA | AGG | 302 |
| 1235 | -1 | CACAAAAAAGGTCACACCTT | TGG | 303 |
| 1247 | 1 | CAAAGGTGTGACCTTTTTTG | TGG | 304 |
| 1247 | -1 | GATAAGAAAAACCACAAAAA | AGG | 305 |
| 1317 | 1 | ATGTTTTAAGTGTTTATGTT | AGG | 306 |
| 1321 | 1 | TTTAAGTGTTTATGTTAGGT | TGG | 307 |
| 1342 | 1 | GGTTTGATTGCTGCAAATGA | TGG | 308 |
| 1369 | -1 | TCTTGAGAATTCTTGGAATA | TGG | 309 |
| 1376 | -1 | AAATGTTTCTTGAGAATTCT | TGG | 310 |
| 1392 | 1 | AATTCTCAAGAAACATTTCA | AGG | 311 |
| 1393 | 1 | ATTCTCAAGAAACATTTCAA | GGG | 312 |
| 1394 | 1 | TTCTCAAGAAACATTTCAAG | GGG | 313 |
| 1434 | 1 | TCTTCTTGATTTGTTTAATG | AGG | 314 |
| 1473 | 1 | GATTGTAGTTTAGAGCAAAA | TGG | 315 |
| 1501 | 1 | TTTTTGTGTGATTTGTGTGA | CGG | 316 |
| 1519 | 1 | GACGGTTTGCTTTTTCGAAT | AGG | 317 |
| 1538 | -1 | TCATTTGTCCTGAGGCTGTT | TGG | 318 |
| 1541 | 1 | GTTGAATTCCAAACAGCCTC | AGG | 319 |
| 1546 | -1 | CAAATCAATCATTTGTCCTG | AGG | 320 |
| 1573 | -1 | ATCTTTCTCTCCTTCAATTG | TGG | 321 |
| 1574 | 1 | GATTTGATCACCACAATTGA | AGG | 322 |
| 1614 | -1 | TCTAAATATTTCACTTACAG | TGG | 323 |
| 1660 | 1 | ATTCAATCGAAATTTCGAGT | TGG | 324 |
| 1706 | -1 | TCTTGTACTGAACAATTCTA | TGG | 325 |
| 1744 | 1 | TTACTACTCATTCTACCTTC | CGG | 326 |
| 1748 | -1 | ATGGTTTTTTTCATACCGGA | AGG | 327 |
| 1752 | -1 | GGCAATGGTTTTTTTCATAC | CGG | 328 |
| 1767 | -1 | ATTAGAAACAATCTAGGCAA | TGG | 329 |
| 1773 | -1 | AACTCGATTAGAAACAATCT | AGG | 330 |
| 1793 | 1 | TTTCTAATCGAGTTTTTGAT | AGG | 331 |
| 1794 | 1 | TTCTAATCGAGTTTTTGATA | GGG | 332 |
| 1841 | 1 | CTTGAACACTATTTATGAAT | AGG | 333 |
| 1856 | 1 | TGAATAGGTTGCTTGTGCAT | TGG | 334 |
| 1862 | 1 | GGTTGCTTGTGCATTGGTTA | TGG | 335 |
| 1866 | 1 | GCTTGTGCATTGGTTATGGC | TGG | 336 |
| 1893 | -1 | GAATGTTCTTGACATCAACA | TGG | 337 |
| 1916 | 1 | CAAGAACATTCTTATCGAAA | TGG | 338 |
| 1917 | 1 | AAGAACATTCTTATCGAAAT | GGG | 339 |
| 1931 | -1 | ACTCACCTGTACTTGAAAAT | _{A}GG | 340 |
| 1937 | 1 | GGGAACCTATTTTCAAGTAC | _{A}GG | 341 |
| 1948 | 1 | TTCAAGTACAGGTGAGTTGA | TGG | 342 |
| 1960 | -1 | AAAAAGTTCAGTAACAAATG | AGG | 343 |
| 2008 | -1 | CCTACAATATAATATGTCAT | TGG | 344 |
| 2019 | 1 | CCAATGACATATTATATTGT | AGG | 345 |
| 2031 | 1 | TATATTGTAGGATGACTATT | TGG | 346 |
| 2041 | 1 | GATGACTATTTGGATTGTTT | TGG | 347 |
| 2053 | -1 | CCTTGCCAATTACATCTGGG | TGG | 348 |
| 2056 | -1 | ATACCTTGCCAATTACATCT | GGG | 349 |
| 2057 | -1 | CATACCTTGCCAATTACATC | TGG | 350 |
| 2059 | 1 | TTTGGCCACCCAGATGTAAT | TGG | 351 |
| 2064 | 1 | CCACCCAGATGTAATTGGCA | AGG | 352 |
| 2100 | -1 | GTTCCCAACTGAATCAAACT | TGG | 353 |
| 2107 | 1 | TTTGCCAAGTTTGATTCAGT | TGG | 354 |
| 2108 | 1 | TTGCCAAGTTTGATTCAGTT | GGG | 355 |
| 2118 | 1 | TGATTCAGTTGGGAACTTTT | CGG | 356 |
| 2142 | -1 | ACCAATCTGATAATCGAAAA | GGG | 357 |
| 2143 | -1 | TACCAATCTGATAATCGAAA | AGG | 358 |
| 2152 | 1 | GCCCTTTTCGATTATCAGAT | TGG | 359 |
| 2183 | 1 | TTGAAGACTTCAAATGCTCT | TGG | 360 |
| 2187 | 1 | AGACTTCAAATGCTCTTGGT | TGG | 361 |
| 2223 | -1 | TAATAGCTTCTTTTGTTCAT | CGG | 362 |
| 2254 | -1 | CATTTTCATATGAAACGATT | TGG | 363 |
| 2323 | 1 | GTTTGTATTCTGTGTTTTCC | AGG | 364 |
| 2330 | -1 | CTGCTTTGCCATAATGCTCC | TGG | 365 |
| 2333 | 1 | TGTGTTTTCCAGGAGCATTA | TGG | 366 |
| 2395 | 1 | ATATAAAACTCTTGATCTTG | AGG | 367 |
| 2439 | -1 | ACTCGAAAAAAAAAAAAACA | TGG | 368 |
| 2456 | 1 | TTTTTTTTTTTTCGAGTTTG | TGG | 369 |
| 2473 | -1 | GAAAAATCGAATTTAGTAAA | GGG | 370 |
| 2474 | -1 | CGAAAAATCGAATTTAGTAA | AGG | 371 |
| 2486 | 1 | CTTTACTAAATTCGATTTTT | CGG | 372 |
| 2499 | 1 | GATTTTTCGGTTTTGTTTGC | AGG | 373 |
| 2500 | 1 | ATTTTTCGGTTTTGTTTGCA | GGG | 374 |
| 2542 | -1 | CAATCGATTTATTAAGCTTT | TGG | 375 |
| 2572 | -1 | CAGCTTGAACTTCTTTTTTC | GGG | 376 |
| 2573 | -1 | ACAGCTTGAACTTCTTTTTT | CGG | 377 |
| 2601 | 1 | AGCTGTGCTCAAATCTTTCT | TGG | 378 |
| 2618 | 1 | TCTTGGCTAAAATCTACAAA | AGG | 379 |
| 2692 | 1 | CTTTCACTCTTTTTAATAAA | AGG | 380 |
| 2693 | 1 | TTTCACTCTTTTTAATAAAA | GGG | 381 |
| 2716 | 1 | TAACTTTTAGTAATTGTTTT | TGG | 382 |
| 2778 | -1 | AATATCCACCACACTTAGTA | GGG | 383 |
| 2779 | -1 | AAATATCCACCACACTTAGT | AGG | 384 |
| 2781 | 1 | CTTACTTACCCTACTAAGTG | TGG | 385 |
| 2784 | 1 | ACTTACCCTACTAAGTGTGG | TGG | 386 |
| 2817 | 1 | GTAATATCATGTGTTTTCTT | TGG | 387 |
| 2872 | -1 | CAAAAACAAAAAGAGAGAAA | AGG | 388 |
| 2907 | -1 | AACAAATCTTTTGTGAACTT | GGG | 389 |
| 2908 | -1 | AAACAAATCTTTTGTGAACT | TGG | 390 |

TABLE 3
gRNA and complementing PAM sequences of CsGPPS1

| Position | | | | SEQ |
|---|---|---|---|---|
| in SEQ | | | | ID |
| ID NO: 7 | Strand | Sequence | PAM | NO |
| 10 | -1 | ATTATTATATTAAACTATAT | GGG | 391 |
| 11 | -1 | AATTATTATATTAAACTATA | TGG | 392 |
| 28 | 1 | AGTTTAATATAATAATTTTT | AGG | 393 |
| 51 | 1 | AGTATAACTAGCTAATTACA | AGG | 394 |
| 66 | 1 | TTACAAGGCGACATGTCTTA | AGG | 395 |
| 67 | 1 | TACAAGGCGACATGTCTTAA | GGG | 396 |
| 88 | -1 | TTTTTTTTGTATTGAACGAG | TGG | 397 |
| 113 | -1 | GCATATAAGAAAGGTATACT | TGG | 398 |
| 122 | -1 | ACTTACGAGGCATATAAGAA | AGG | 399 |
| 135 | -1 | TGCCTTGGTCGTTACTTACG | AGG | 400 |
| 144 | 1 | TGCCTCGTAAGTAACGACCA | AGG | 401 |
| 150 | -1 | GTCATGGGATTTCATTGCCT | TGG | 402 |
| 165 | -1 | TTATGCTATAATTTAGTCAT | GGG | 403 |
| 166 | -1 | ATTATGCTATAATTTAGTCA | TGG | 404 |
| 197 | -1 | AGGTTTTTGGCTTTTTTTTT | TGG | 405 |
| 210 | -1 | TATTTATTATGTTAGGTTTT | TGG | 406 |
| 217 | -1 | AATGTATTATTTATTATGTT | AGG | 407 |
| 257 | 1 | TTCAATGTCAAACAAAAAAA | CGG | 408 |
| 293 | -1 | TGTTTTTAAAACAAATTTGG | GGG | 409 |
| 294 | -1 | GTGTTTTTAAAACAAATTTG | GGG | 410 |
| 295 | -1 | TGTGTTTTTAAAACAAATTT | GGG | 411 |
| 296 | -1 | ATGTGTTTTTAAAACAAATT | TGG | 412 |
| 325 | -1 | AAAGAAAGTAAGGAAAGCAA | TGG | 413 |
| 335 | -1 | TTATATAAATAAAGAAAGTA | AGG | 414 |
| 357 | 1 | TTATTTATATAATTTTTTTT | AGG | 415 |
| 358 | 1 | TATTTATATAATTTTTTTTA | GGG | 416 |
| 359 | 1 | ATTTATATAATTTTTTTTAG | GGG | 417 |
| 381 | 1 | GAGCTCTAGAGCTTCATCAA | TGG | 418 |
| 384 | 1 | CTCTAGAGCTTCATCAATGG | CGG | 419 |
| 422 | -1 | TAAACATGATGAACAAATCT | TGG | 420 |
| 449 | -1 | TTGGATTTACATGTGAAATG | TGG | 421 |
| 468 | -1 | TACGTGACTTAACGACTTAT | TGG | 422 |
| 491 | -1 | TTGGACATGGTTATTCTCAT | GGG | 423 |
| 492 | -1 | TTTGGACATGGTTATTCTCA | TGG | 424 |
| 504 | -1 | ATGATGATGCTGTTTGGACA | TGG | 425 |
| 510 | -1 | ATAAGAATGATGATGCTGTT | TGG | 426 |
| 537 | -1 | ATCTACATCGGCTGTTGTGG | AGG | 427 |
| 540 | -1 | GGCATCTACATCGGCTGTTG | TGG | 428 |
| 549 | -1 | CTTGAGATGGGCATCTACAT | CGG | 429 |
| 561 | -1 | AGTGATGGATTGCTTGAGAT | GGG | 430 |
| 562 | -1 | TAGTGATGGATTGCTTGAGA | TGG | 431 |
| 576 | -1 | GAGTGGTGGCTTGATAGTGA | TGG | 432 |
| 590 | -1 | GCCTCGTGAACTGAGAGTGG | TGG | 433 |
| 593 | -1 | ATGGCCTCGTGAACTGAGAG | TGG | 434 |
| 600 | 1 | GCCACCACTCTCAGTTCACG | AGG | 435 |
| 612 | -1 | GGAAAAGATGAAATTGTACA | TGG | 436 |
| 633 | -1 | CGGTGCTAAATTCGGAGGTG | TGG | 437 |
| 638 | -1 | AATGACGGTGCTAAATTCGG | AGG | 438 |
| 641 | -1 | CACAATGACGGTGCTAAATT | CGG | 439 |
| 653 | -1 | CACGCCGCCACGCACAATGA | CGG | 440 |
| 657 | 1 | TTTAGCACCGTCATTGTGCG | TGG | 441 |
| 660 | 1 | AGCACCGTCATTGTGCGTGG | CGG | 442 |
| 676 | 1 | GTGGCGGCGTGTGAGCTTGT | CGG | 443 |
| 677 | 1 | TGGCGGCGTGTGAGCTTGTC | GGG | 444 |
| 678 | 1 | GGCGGCGTGTGAGCTTGTCG | GGG | 445 |
| 679 | 1 | GCGGCGTGTGAGCTTGTCGG | GGG | 446 |
| 687 | 1 | TGAGCTTGTCGGGGGCCACC | AGG | 447 |
| 691 | -1 | CTGCCATGGCCTGGTCCTGG | TGG | 448 |
| 693 | 1 | TGTCGGGGGCCACCAGGACC | AGG | 449 |
| 694 | -1 | CTGCTGCCATGGCCTGGTCC | TGG | 450 |
| 699 | 1 | GGGCCACCAGGACCAGGCCA | TGG | 451 |
| 700 | -1 | CGGAGGCTGCTGCCATGGCC | TGG | 452 |
| 705 | -1 | CAAGGCGGAGGCTGCTGCCA | TGG | 453 |
| 717 | -1 | GTGGATGACGCGCAAGGCGG | AGG | 454 |
| 720 | -1 | TGCGTGGATGACGCGCAAGG | CGG | 455 |
| 723 | -1 | GGCTGCGTGGATGACGCGCA | AGG | 456 |
| 736 | -1 | CATGAGTGAAGATGGCTGCG | TGG | 457 |
| 744 | -1 | GAGGTGGTCATGAGTGAAGA | TGG | 458 |
| 760 | -1 | GCCTGCCCGTTAAAGGGAGG | TGG | 459 |
| 763 | -1 | TGGGCCTGCCCGTTAAAGGG | AGG | 460 |
| 765 | 1 | TCATGACCACCTCCCTTTAA | CGG | 461 |
| 766 | 1 | CATGACCACCTCCCTTTAAC | GGG | 462 |
| 766 | -1 | GATTGGGCCTGCCCGTTAAA | GGG | 463 |
| 767 | -1 | GGATTGGGCCTGCCCGTTAA | AGG | 464 |
| 770 | 1 | ACCACCTCCCTTTAACGGGC | AGG | 465 |
| 782 | -1 | GCCTCAGGACTTGTTGGATT | GGG | 466 |
| 783 | -1 | TGCCTCAGGACTTGTTGGAT | TGG | 467 |
| 788 | -1 | GTCGCTGCCTCAGGACTTGT | TGG | 468 |
| 792 | 1 | GCCCAATCCAACAAGTCCTG | AGG | 469 |
| 797 | -1 | GAATTGTGGGTCGCTGCCTC | AGG | 470 |
| 810 | -1 | ATTTGGGTTGTAAGAATTGT | GGG | 471 |
| 811 | -1 | TATTTGGGTTGTAAGAATTG | TGG | 472 |
| 826 | -1 | GGAGAAGGAGCTGAATATTT | GGG | 473 |
| 827 | -1 | GGGAGAAGGAGCTGAATATT | TGG | 474 |
| 840 | 1 | AAATATTCAGCTCCTTCTCC | CGG | 475 |
| 841 | -1 | GTACAATTGCGTCCGGGAGA | AGG | 476 |
| 847 | -1 | CAAAAGGTACAATTGCGTCC | GGG | 477 |
| 848 | -1 | CCAAAAGGTACAATTGCGTC | CGG | 478 |
| 859 | 1 | CCGGACGCAATTGTACCTTT | TGG | 479 |
| 860 | 1 | CGGACGCAATTGTACCTTTT | GGG | 480 |
| 863 | -1 | GCCAACAATTCGAACCCAAA | AGG | 481 |
| 873 | 1 | ACCTTTTGGGTTCGAATTGT | TGG | 482 |
| 885 | -1 | ATGGGTAAGGTCATCAGAAT | TGG | 483 |
| 898 | -1 | GATCTGATTTATTATGGGTA | AGG | 484 |
| 903 | -1 | AATCCGATCTGATTTATTAT | GGG | 485 |
| 904 | -1 | AAATCCGATCTGATTTATTA | TGG | 486 |
| 911 | 1 | TTACCCATAATAAATCAGAT | CGG | 487 |
| 920 | 1 | ATAAATCAGATCGGATTTTG | CGG | 488 |
| 921 | 1 | TAAATCAGATCGGATTTTGC | GGG | 489 |
| 949 | 1 | GTAGAGTTCACACGCACCTT | TGG | 490 |
| 954 | -1 | AATAGTTCCTCGTGATCCAA | AGG | 491 |
| 958 | 1 | ACACGCACCTTTGGATCACG | AGG | 492 |
| 988 | -1 | ATCTACTGGCTAGCTTCTCA | TGG | 493 |
| 1002 | -1 | ACTATCAACGTCAAATCTAC | TGG | 494 |
| 1032 | -1 | ATGGCCCCACCCGACAGTTT | TGG | 495 |
| 1033 | 1 | AGTCATGAAGCCAAAACTGT | CGG | 496 |
| 1034 | 1 | GTCATGAAGCCAAAACTGTC | GGG | 497 |
| 1037 | 1 | ATGAAGCCAAAACTGTCGGG | TGG | 498 |
| 1038 | 1 | TGAAGCCAAAACTGTCGGGT | GGG | 499 |
| 1039 | 1 | GAAGCCAAAACTGTCGGGTG | GGG | 500 |
| 1051 | -1 | CCTTCTTCAAAGAGGGATAA | TGG | 501 |
| 1058 | -1 | GCACCTTCCTTCTTCAAAGA | GGG | 502 |
| 1059 | -1 | CGCACCTTCCTTCTTCAAAG | AGG | 503 |
| 1062 | 1 | CCATTATCCCTCTTTGAAGA | AGG | 504 |
| 1066 | 1 | TATCCCTCTTTGAAGAAGGA | AGG | 505 |
| 1096 | 1 | CATGCATGCGCTGCTGCATG | TGG | 506 |
| 1097 | 1 | ATGCATGCGCTGCTGCATGT | GGG | 507 |
| 1098 | 1 | TGCATGCGCTGCTGCATGTG | GGG | 508 |
| 1108 | 1 | GCTGCATGTGGGGCCATTCT | TGG | 509 |
| 1110 | -1 | TTCATGTGCCTCTCCAAGAA | TGG | 510 |
| 1113 | 1 | ATGTGGGGCCATTCTTGGAG | AGG | 511 |
| 1128 | 1 | TGGAGAGGCACATGAAGAAG | AGG | 512 |
| 1150 | 1 | GTTGAGAAGTTGAGAACTTT | TGG | 513 |
| 1161 | 1 | GAGAACTTTTGGTCTTTATG | TGG | 514 |
| 1162 | 1 | AGAACTTTTGGTCTTTATGT | GGG | 515 |
| 1174 | 1 | CTTTATGTGGGCATGATTCA | AGG | 516 |
| 1191 | -1 | GCTGCTCATTATAAATCTAT | TGG | 517 |
| 1239 | 1 | AGAAGCAGATAGAATCATCG | AGG | 518 |
| 1254 | 1 | CATCGAGGAGTTAACCAATT | TGG | 519 |
| 1257 | -1 | TAGTTCCTGGCGAGCCAAAT | TGG | 520 |
| 1263 | 1 | GTTAACCAATTTGGCTCGCC | AGG | 521 |
| 1270 | -1 | CATCGAAATATTTTAGTTCC | TGG | 522 |
| 1282 | 1 | CAGGAACTAAAATATTTCGA | TGG | 523 |
| 1283 | 1 | AGGAACTAAAATATTTCGAT | GGG | 524 |
| 1307 | -1 | CGAAAAAGAAAGGTTGAAAA | TGG | 525 |
| 1317 | -1 | TTTCTATAGACGAAAAAGAA | AGG | 526 |
| 1396 | 1 | TTTATTTGAAACTAGAAAAC | TGG | 527 |
| 1418 | -1 | CTTAATTAGACTAGCTATGT | AGG | 528 |
| 1573 | -1 | AAAATTTCTTAAAAATTATA | AGG | 529 |
| 1615 | 1 | AGTAGCAAAAATTAAACTTT | TGG | 530 |

TABLE 4
gRNA and complementing PAM sequences of CsGPPS2

| Position | | | | SEQ |
|---|---|---|---|---|
| in SEQ | | | | ID |
| ID NO: 10 | Strand | Sequence | PAM | NO |
| 37 | 1 | GCATCAATCTTAAGTTTTTG | AGG | 531 |
| 56 | -1 | TAAAAAATTAGGGATAATTG | CGG | 532 |
| 66 | -1 | TACGTTCATATAAAAAATTA | GGG | 533 |
| 67 | -1 | TTACGTTCATATAAAAAATT | AGG | 534 |
| 115 | -1 | ACAACATCAATTATTATTTT | TGG | 535 |
| 177 | -1 | ATAATAATTTTTTCTTCAAG | GGG | 536 |
| 178 | -1 | TATAATAATTTTTTCTTCAA | GGG | 537 |
| 179 | -1 | CTATAATAATTTTTTCTTCA | AGG | 538 |
| 231 | -1 | AGATACAATAAAGTGGGACA | TGG | 539 |
| 237 | -1 | TGAAGAAGATACAATAAAGT | GGG | 540 |
| 238 | -1 | TTGAAGAAGATACAATAAAG | TGG | 541 |
| 283 | 1 | CAAAAATTATACACTAAGAT | TGG | 542 |
| 295 | -1 | TTTTATTATTATTTATCAAA | TGG | 543 |
| 317 | 1 | ATAATAATAAAAAAAATCTA | TGG | 544 |
| 318 | 1 | TAATAATAAAAAAAATCTAT | GGG | 545 |
| 330 | -1 | GAAATTTCAAGCATTATTCT | AGG | 546 |
| 358 | -1 | AGAACATTTCAAGGGAAGAA | GGG | 547 |
| 359 | -1 | TAGAACATTTCAAGGGAAGA | AGG | 548 |
| 366 | -1 | AAAGAATTAGAACATTTCAA | GGG | 549 |
| 367 | -1 | AAAAGAATTAGAACATTTCA | AGG | 550 |
| 391 | 1 | TAATTCTTTTATAGCTAATT | TGG | 551 |
| 409 | -1 | GGAGAGACTAAAAAGAGTTG | AGG | 552 |
| 430 | -1 | AATTGGTAGAGGGAAAGAAG | AGG | 553 |
| 440 | -1 | GATATTCTAAAATTGGTAGA | GGG | 554 |
| 441 | -1 | GGATATTCTAAAATTGGTAG | AGG | 555 |
| 447 | -1 | ATTCAAGGATATTCTAAAAT | TGG | 556 |
| 462 | -1 | CTCTATGTGGGCATGATTCA | AGG | 557 |
| 474 | -1 | AGCAAGTTTGGTCTCTATGT | GGG | 558 |
| 475 | -1 | GAGCAAGTTTGGTCTCTATG | TGG | 559 |
| 486 | -1 | GAAGAAAAATTGAGCAAGTT | TGG | 560 |
| 523 | -1 | ATGTGGTGCCATTCTTGGAG | GGG | 561 |
| 524 | -1 | CATGTGGTGCCATTCTTGGA | GGG | 562 |
| 525 | -1 | ACATGTGGTGCCATTCTTGG | AGG | 563 |
| 526 | 1 | TTCATTTGCCCCTCCAAGAA | TGG | 564 |
| 528 | -1 | GCTACATGTGGTGCCATTCT | TGG | 565 |
| 540 | -1 | TATGCGTGCGCGGCTACATG | TGG | 566 |
| 550 | -1 | AGGGAAGTTGTATGCGTGCG | CGG | 567 |
| 569 | -1 | ACACATGTCGAAAAAAGGAA | GGG | 568 |
| 570 | -1 | TACACATGTCGAAAAAAGGA | AGG | 569 |
| 574 | -1 | CGATTACACATGTCGAAAAA | AGG | 570 |
| 599 | -1 | AAGAAAACAATAATGCTGAT | TGG | 571 |
| 622 | 1 | ATTGTTTTCTTCCTCACCAT | TGG | 572 |
| 622 | -1 | AGTCAATAGATCCAATGGTG | AGG | 573 |
| 627 | -1 | AAGGTAGTCAATAGATCCAA | TGG | 574 |
| 645 | 1 | ATCTATTGACTACCTTCTCA | TGG | 575 |
| 646 | -1 | TGATGGTCAATTCCATGAGA | AGG | 576 |
| 663 | -1 | GGATCACAAGGGATTATTGA | TGG | 577 |
| 674 | -1 | CGCGAGCCTTTGGATCACAA | GGG | 578 |
| 675 | -1 | ACGCGAGCCTTTGGATCACA | AGG | 579 |
| 679 | 1 | AATAATCCCTTGTGATCCAA | AGG | 580 |
| 684 | -1 | GTGGAGATCACGCGAGCCTT | TGG | 581 |
| 703 | -1 | TCGGGTTTTGAAGGTTATTG | TGG | 582 |
| 712 | -1 | TCATGCAGATCGGGTTTTGA | AGG | 583 |
| 721 | -1 | CGAAGATGATCATGCAGATC | GGG | 584 |
| 722 | -1 | TCGAAGATGATCATGCAGAT | CGG | 585 |
| 737 | 1 | TCTGCATGATCATCTTCGAT | CGG | 586 |
| 738 | 1 | CTGCATGATCATCTTCGATC | GGG | 587 |
| 750 | 1 | CTTCGATCGGGTTATCTAAA | CGG | 588 |
| 751 | 1 | TTCGATCGGGTTATCTAAAC | GGG | 589 |
| 773 | 1 | GTTAACAACTCACACCCGAA | AGG | 590 |
| 774 | 1 | TTAACAACTCACACCCGAAA | GGG | 591 |
| 776 | -1 | CAGATGCAATAGTCCCTTTC | GGG | 592 |
| 777 | -1 | CCAGATGCAATAGTCCCTTT | CGG | 593 |
| 788 | 1 | CCGAAAGGGACTATTGCATC | TGG | 594 |
| 789 | 1 | CGAAAGGGACTATTGCATCT | GGG | 595 |
| 809 | 1 | GGGATAAGAAGCTCAATATT | TGG | 596 |
| 822 | 1 | CAATATTTGGATTGTAAGCG | TGG | 597 |
| 833 | 1 | TTGTAAGCGTGGTGAATCAT | TGG | 598 |
| 839 | 1 | GCGTGGTGAATCATTGGATT | TGG | 599 |
| 844 | 1 | GTGAATCATTGGATTTGGAT | TGG | 600 |
| 853 | 1 | TGGATTTGGATTGGATCTAT | CGG | 601 |
| 860 | 1 | GGATTGGATCTATCGGTTAA | AGG | 602 |
| 864 | 1 | TGGATCTATCGGTTAAAGGA | AGG | 603 |
| 897 | 1 | TAAAGCTAGCTACATGCATG | AGG | 604 |
| 910 | 1 | ATGCATGAGGTGCAAGCTCG | AGG | 605 |
| 922 | 1 | CAAGCTCGAGGCTGCTGCCA | CGG | 606 |
| 928 | -1 | GGGCCACAGGAGGCAAGCCG | TGG | 607 |
| 936 | 1 | CTGCCACGGCTTGCCTCCTG | TGG | 608 |
| 938 | -1 | AACTTGTTGGGGGCCACAGG | AGG | 609 |
| 941 | -1 | GTGAACTTGTTGGGGGCCAC | AGG | 610 |
| 948 | -1 | GCGGCGTGTGAACTTGTTGG | GGG | 611 |
| 949 | -1 | GGCGGCGTGTGAACTTGTTG | GGG | 612 |
| 950 | -1 | TGGCGGCGTGTGAACTTGTT | GGG | 613 |
| 951 | -1 | GTGGCGGCGTGTGAACTTGT | TGG | 614 |
| 967 | -1 | AGCACCTTTGCTATGTGTGG | CGG | 615 |
| 970 | -1 | TTCAGCACCTTTGCTATGTG | TGG | 616 |
| 974 | 1 | CACGCCGCCACACATAGCAA | AGG | 617 |
| 986 | 1 | CATAGCAAAGGTGCTGAAGT | TGG | 618 |
| 989 | 1 | AGCAAAGGTGCTGAAGTTGG | CGG | 619 |
| 1000 | 1 | TGAAGTTGGCGGCGTTGTAA | AGG | 620 |
| 1012 | -1 | CTATGAGCCCATGTACAATT | TGG | 621 |
| 1015 | 1 | TGTAAAGGCCAAATTGTACA | TGG | 622 |
| 1016 | 1 | GTAAAGGCCAAATTGTACAT | GGG | 623 |
| 1034 | 1 | ATGGGCTCATAGACTGTGAA | AGG | 624 |
| 1037 | 1 | GGCTCATAGACTGTGAAAGG | AGG | 625 |
| 1051 | 1 | GAAAGGAGGCTTGACAATGA | TGG | 626 |
| 1065 | 1 | CAATGATGGATTGCTTGAGA | TGG | 627 |
| 1066 | 1 | AATGATGGATTGCTTGAGAT | GGG | 628 |
| 1078 | -1 | CTCTATAACAAAAGATATAG | AGG | 629 |
| 1090 | 1 | CTCTATATCTTTTGTTATAG | AGG | 630 |
| 1132 | 1 | ATGATGTTGAAAATTTTGAG | AGG | 631 |
| 1138 | 1 | TTGAAAATTTTGAGAGGACA | TGG | 632 |
| 1151 | 1 | GAGGACATGGTGATTGTCAT | AGG | 633 |
| 1183 | 1 | AAAATTAGATGACATTGATG | AGG | 634 |
| 1191 | 1 | ATGACATTGATGAGGAGAGA | TGG | 635 |
| 1196 | 1 | ATTGATGAGGAGAGATGGTG | TGG | 636 |
| 1217 | 1 | GGAGAGCTAGAGAGAAATTA | AGG | 637 |
| 1231 | 1 | AAATTAAGGAAATATATATA | AGG | 638 |
| 1240 | 1 | AAATATATATAAGGAAGTAA | TGG | 639 |
| 1250 | 1 | AAGGAAGTAATGGAGTAAAT | AGG | 640 |
| 1260 | 1 | TGGAGTAAATAGGCAATTAT | TGG | 641 |
| 1291 | -1 | TTTGAAAAGAAATTGATTGA | AGG | 642 |
| 1338 | 1 | GAGCATTGTTATTGAAGATC | AGG | 643 |
| 1354 | 1 | GATCAGGTGACATTTTCAAT | TGG | 644 |
| 1427 | -1 | TTCCAATATTATATTGTTAT | CGG | 645 |
| 1436 | 1 | TACCGATAACAATATAATAT | TGG | 646 |

Cannabis plants were transformed using Agrobacterium or biolistics (gene gun) methods. For Agrobacterium and bioloistics a DNA plasmid carrying Cas9+gene specific gRNA was used. A vector containing a selection marker, Cas9 gene and relevant gene specific gRNA's was constructed. For biolistics, Ribonucleoprotein (RNP) complexes carrying Cas9 protein+gene specific gRNA were used. RNP complexes were created by mixing the Cas9 protein with relevant gene specific gRNA's.

Reference is made to Table 5 presenting a summary of the sequences and corresponding SEQ ID Nos within the scope of the current invention.

TABLE 5
Summary of sequences within the scope of the present invention

| | | Coding | | |
|---|---|---|---|---|
| Sequence | Genomic | sequence | Amino acid | gRNA |
| name | sequence | (CDS) | sequence | sequences |
| CsFPPS1 | SEQ ID | SEQ ID | SEQ ID | SEQ ID |
| | NO: 1 | NO: 2 | NO: 3 | NO: 13-237 |
| CsFPPS2 | SEQ ID | SEQ ID | SEQ ID | SEQ ID |
| | NO: 4 | NO: 5 | NO: 6 | NO: 238-390 |
| CsGPPS1 | SEQ ID | SEQ ID | SEQ ID | SEQ ID |
| | NO: 7 | NO: 8 | NO: 9 | NO: 391-530 |
| CsGPPS2 | SEQ ID | SEQ ID | SEQ ID | SEQ ID |
| | NO: 10 | NO: 11 | NO: 12 | NO: 530-646 |

Transformed Cannabis plants with genome edited versions of the aforementioned targeted Cannabis terpene synthesis genes CsFPPS1, CsFPPS2, CsGPPS1 and CsGPPS2, were selected. These plants were further examined for reduced expression (at the transcription and post transcription levels) of these genes. In addition, transformed Cannabis plants phenotypically presenting reduced odor emission, using a protocol established by the present invention, were selected.

Reference is now made to Table 6 presenting non-limiting examples of Cannabis terpene synthesis (CsTPS) genes within the scope of the present invention (Booth et al., 2017, incorporated herein by reference). The table encompass sequences from various Cannabis strains, and of all stages of terpene biosynthesis including mono- and sesqui-TPS, whose products comprise major compounds such as β-myrcene, (E)-β-ocimene, (−)-limonene, (+)-α-pinene, β-caryophyllene, and α-humulene. The CsTPS gene family offer opportunities for silencing by genome editing selected terpene synthesis genes to modulate terpene profiles to significantly reduce or eliminate emission of undesirable odor in different Cannabis strains and varieties.

TABLE 6
List of terpene synthesis genes in the Cannabis plant
GeneBank accession numbers for genomic
regions containing putative terpene synthases
from Purple Kush

| | |
|---|---|
| CsTPS1PK | KY624372 |
| CsTPS4PK | KY624361 |
| CsTPS5PK | KY624374 |
| CsTPS6PK | KY624363 |
| CsTPS7PK | KY624368 |
| CsTPS8PK | KY624352 |
| CsTPS9PK | KY624366 |
| CsTPS10PK | KY624347 |
| CsTPS11PK | KY624348 |
| CsTPS12PK | KY624349 |
| CsTPS13PK | KY624350 |
| CsTPS14PK | KY624351 |
| CsTPS15PK | KY624353, |
| CsTPS16PK | KY624354 |
| CsTPS17PK | KY624355 |
| CsTPS18PK | KY624356 |
| CsTPS19PK | KY624357 |
| CsTPS20PK | KY624358 |
| CsTPS21PK | KY624360 |
| CsTPS22PK | KY624360 |
| CsTPS23PK | KY624362 |
| CsTPS24PK | KY624364 |
| CsTPS25PK | KY624364 |
| CsTPS26PK | KY624365 |
| CsTPS27PK | KY624365 |
| CsTPS30PK | KY624367 |
| CsTPS31PK | KY624369 |
| CsTPS32PK | KY624370 |
| CsTPS33PK | KY624371 |
| CsTPS34PK | KY624373 |
| CsTPS35PK | KY624375 |
| CsTPS12PK | KY014559 |
| CsTPS13PK | KY014558 |

Accession numbers for terpene synthase genomic
regions from ‘Finola’

| | |
|---|---|
| CsTPS1FN | KY014557 |
| CsTPS2FN | KY014565 |
| CsTPS3FN | KY014561 |
| CsTPS4FN | KY014564 |
| CsTPS5FN | KY014560 |
| CsTPS6FN | KY014563 |
| CsTPS7FN | KY014554 |
| CsTPS8FN | KY014556 |
| CsTPS9FN | KY014555 |
| CsTPS11FN | KY014562 |

Accession numbers for genes in the
methylerythritol phosphate (MEP) pathway

| | |
|---|---|
| CsDXSl | KY014576 |
| CsDXS2 | KY014577 |
| CsDXR | KY014568 |
| CsMCT | KY014578 |
| CsCMK | KY014575 |
| CsHDS | KY014570 |
| CsHDR | KY014579 |

Accession numbers for genes in the mevalonic
acid or mevalonate (MEV) pathway

| | |
|---|---|
| CsHMGS | KY014582 |
| CsHMGR1 | KY014572 |
| CsHMGR2 | KY014553 |
| CsMK | KY014574 |
| CsPMK | KY014581 |
| CsMPDC | KY014566 |
| CsIDI | KY014569 |

### REFERENCES

Booth, J. K., Page, J. E., and Bohlmann, J. (2017). Terpene synthases from Cannabis sativa. PLOS ONE 12, e0173911.
Public Health Ontario (2018). Evidence Brief: Odours from Cannabis Production.
USDA, Washington, D.C., Mar. 28, 2018 Secretary Perdue Issues USDA Statement on Plant Breeding Innovation.
Xie, K., and Yinong Y. (2013). RNA-guided genome editing in plants using a CRISPR-Cas system. Molecular plant 6.6: 1975-1983.
Krill C., Rochfort S., and Spangenberg G. (2020). A High-Throughput Method for the Comprehensive Analysis of Terpenes and Terpenoids in Medicinal Cannabis Biomass. Metabolites, 10, 276: 1-14

1.-75. (canceled)

76. A modified Cannabis plant exhibiting reduced volatile organic compounds (VOCs) emission, wherein said modified plant comprises at least one targeted gene modification conferring reduced expression or silencing of at least one gene involved in a terpene biosynthesis pathway.

77. The modified Cannabis plant according to claim 76, wherein at least one of the following holds true:
a. said at least one targeted gene modification confers reduced expression or silencing of at least one gene involved in a terpene biosynthesis pathway as compared to a Cannabis plant lacking said targeted gene modification;
b. said terpene biosynthesis pathway is selected from methylerythritol phosphate (MEP) pathway, mevalonic acid or mevalonate (MEV) pathway, isoprenoid biosynthetic pathway, formation of GPP, FPP and GGPP pathways, formation of squalene pathway, formation of Mono-, Sesqui- and Di-Terpenes pathways, formation of triterpenes from squalene pathway and any combination thereof; and
c. the at least one gene involved in a terpene biosynthesis pathway is selected from CsTPS1PK, CsTPS4PK, CsTPS5PK, CsTPS6PK, CsTPS7PK, CsTPS8PK, CsTPS9PK, CsTPS10PK, CsTPS11PK, CsTPS12PK, CsTPS13PK, CsTPS14PK, CsTPS15PK, CsTPS16PK, CsTPS17PK, CsTPS18PK, CsTPS19PK, CsTPS20PK, CsTPS21PK, CsTPS22PK, CsTPS23PK, CsTPS24PK, CsTPS25PK, CsTPS26PK, CsTPS27PK, CsTPS30PK, CsTPS31PK, CsTPS32PK, CsTPS33PK, CsTPS34PK, CsTPS35PK, CsTPS12PK, CsTPS13PK, CsTPS1FN, CsTPS2FN, CsTPS3FN, CsTPS4FN, CsTPS5FN, CsTPS6FN, CsTPS7FN, CsTPS8FN, CsTPS9FN, CsTPS11FN, CsDXS1, CsDXS2, CsDXR, CsMCT, CsCMK, CsHDS, CsHDR, CsHMGS, CsHMGR1, CsHMGR2, CsMK, CsPMK, CsMPDC, CsIDI, CsFPPS1, CsFPPS2, CsGPPS1, CsGPPS2 and any combination thereof.

78. The modified Cannabis plant according to claim 77, wherein said gene involved in a terpene biosynthesis pathway is selected from (a) a gene encoding CsFPPS1 characterized by a sequence selected from a sequence comprising at least 75% sequence identity to SEQ ID NO: 1-3, (b) a gene encoding CsFPPS2 characterized by a sequence selected from a sequence comprising at least 75% sequence identity to SEQ ID NO: 4-6, (c) a gene encoding CsGPPS1 characterized by a sequence selected from a sequence comprising at least 75% sequence identity to SEQ ID NO: 7-9, (d) a gene encoding CsGPPS2 characterized by a sequence selected from a sequence comprising at least 75% sequence identity to SEQ ID NO: 10-12, and (e) any combination thereof.

79. The modified Cannabis plant according to claim 76, wherein said gene modification is introduced using CRISPR (Clustered Regularly Interspaced Short Palindromic Repeats) and CRISPR-associated (Cas) gene (CRISPR/Cas) system, Transcription activator-like effector nuclease (TALEN), Zinc Finger Nuclease (ZFN), meganuclease or any combination thereof.

80. The modified Cannabis plant according to claim 76, wherein said targeted gene modification is introduced into the Cannabis plant or a cell thereof using an expression cassette or construct comprising (a) Cas DNA and gRNA sequence selected from the group consisting of SEQ ID NO:13-646 and any combination thereof, or (b) a ribonucleoprotein (RNP) complex comprising Cas protein and gRNA sequence selected from the group consisting of SEQ ID NO:13-646 and any combination thereof, the gRNA targeted for CsFPPS1, CsFPPS2, CsGPPS1 and/or CsGPPS2 comprises a nucleic acid sequence as set forth in SEQ ID NO: 13-237, SEQ ID NO: 238-390, SEQ ID NO: 391-530 and SEQ ID NO: 531-646, respectively.

81. The modified Cannabis plant according to claim 76, wherein, said gene modification is introduced using an expression cassette comprising a) a nucleotide sequence encoding one or more gRNA molecules comprising a DNA sequence which is complementary with a target domain sequence within a gene selected from CsFPPS1, CsFPPS2, CsGPPS1 and CsGPPS2, and b) a nucleotide sequence encoding a Cas molecule, or a Cas protein, the target domain sequence within the Cannabis genome is selected from the group comprising of 1) a nucleic acid sequence encoding the polypeptide of CsFPPS1, the nucleic acid having a sequence as set forth in SEQ ID NO: 1 (2) a nucleic acid sequence encoding the polypeptide of CsFPPS2, the nucleic acid having a sequence as set forth in SEQ ID NO: 4 (3) a nucleic acid sequence encoding the polypeptide of CsGPPS1, the nucleic acid having a sequence as set forth in SEQ ID NO: 7 (4) a nucleic acid sequence encoding the polypeptide of CsGPPS2, the nucleic acid having a sequence as set forth in SEQ ID NO: 10 (5) a nucleic acid sequence having at least 80% sequence identity to at least 200 contiguous nucleotides of the nucleic acid sequence of CsFPPS1, (6) a nucleic acid sequence having at least 80% sequence identity to at least 200 contiguous nucleotides of the nucleic acid sequence of CsFPPS2, (7) a nucleic acid sequence having at least 80% sequence identity to at least 200 contiguous nucleotides of the nucleic acid sequence of CsGPPS1, (8) a nucleic acid sequence having at least 80% sequence identity to at least 200 contiguous nucleotides of the nucleic acid sequence of CsGPPS2.

82. The modified Cannabis plant according to claim 76, wherein at least one of the following holds true:
a. the targeted gene modification is a CRISPR/Cas9-induced heritable mutated allele of at least one of CsFPPS1, CsFPPS2, CsGPPS1 and/or CsGPPS2 encoding gene;
b. the expression of the at least one gene involved in a terpene biosynthesis pathway is eliminated;
c. the modified plant has reduced odor resulting from volatile compounds emission or is odor free or odorless Cannabis plant;
d. the VOCs are selected from essential oils, secondary metabolites, terpenoids, terpenes, oxygenated and any combination thereof;
e. the VOCs comprise at least one of hemiterpenes, monoterpenes, sesquiterpenes, diterpenes, sesterterpenes, triterpenes, tetraterpenes and polyterpenes;
f. the VOCs are selected from pinene, alpha-pinene, beta-pinene, cis-pinane, trans-pinane, cis-pinanol, trans-pinanol, limonene; linalool; myrcene; eucalyptol; a-phellandrene; b-phellandrene; a-ocimene; b-ocimene, cis-ocimene, ocimene, delta-3-carene; fenchol; sabinene, bomeol, isobomeol, camphene, camphor, phellandrene, a-phellandrene, a-terpinene, geraniol, linalool, nerol, menthol, terpinolene, a-terpinolene, b-terpinolene, g-terpinolene, delta-terpinolene, a-terpineol, trans-2-pinanol, caryophyllene, caryophyllene oxide, humulene, a-humulene, a-bisabolene; b-bisabolene; santalol; selinene; nerolidol, bisabolol; a-cedrene, b-cedrene, b-eudesmol, eudesm-7(II)-en-4-ol, selina-3,7(II)-diene, guaiol, valencene, a-guaiene, beta-guaiene, delta-guaiene, guaiene, famesene, a-famesene, b-famesene, elemene, a-elemene, b-elemene, gamma-elemene, delta-elemene, germacrene, germacrene A, germacrene B, germacrene C, germacrene D, germacrene E, oridonin, phytol, isophytol, ursolic acid, oleanolic acid, and/or 1.5 ene compounds, including guaia-I(10),II-diene, and 1.5 ene. Guaia-1(10), 11-diene.isoprene, α-pinene, β-pinene, d-limonene, β-phellandrene, α-terpinene, α-thujene, γ-terpinene, β-myrcene, (E)-β-ocimene, (−)-limonene, (+)-α-pinene, β-caryophyllene, and α-humulene and any combination thereof;
g. said Cannabis plant does not comprise a transgene within its genome; and
h. the gene modification of CsFPPS1, CsFPPS2, CsGPPS1 and/or CsGPPS2 genes does not involve insertion of exogenous genetic material and produces a non-naturally occurring Cannabis plant or cell thereof.

83. A progeny plant, plant part, tissue culture of regenerable cells, protoplasts or callus, plant cell or plant seed of a modified plant according to claim 76.

84. A medical Cannabis product comprising the modified Cannabis plant according to claim 76 or a part or extract thereof.

85. A method for producing a modified Cannabis plant according to claim 76, said method comprises introducing using targeted genome modification, at least one genomic modification conferring reduced expression or silencing of at least one gene involved in a terpene biosynthesis pathway.

86. The method according to claim 85, wherein said method comprises steps of:
a. optionally, introducing using genome editing a loss of function mutation in at least one gene involved in a terpene biosynthesis pathway;
b. identifying at least one Cannabis gene involved in a terpene biosynthesis pathway;
c. designing and/or synthetizing at least one guide RNA (gRNA) comprising a nucleotide sequence corresponding or complementary to a target sequence is said at least one identified Cannabis gene involved in a terpene biosynthesis pathway;
d. transforming a Cannabis plant cells with endonuclease or nucleic acid encoding endonuclease, together with the at least one gRNA or a DNA encoding the gRNA;
e. optionally, culturing said transformed Cannabis cells;
f. selecting Cannabis plant or plant cells thereof carrying induced targeted loss of function mutation in the at least one gene involved in a terpene biosynthesis pathway; and
g. optionally, regenerating a modified Cannabis plant from said transformed plant cell, plant cell nucleus, or plant tissue.

87. The method according to claim 86, further comprises at least one step of:
a. screening the genome of the transformed Cannabis plant or plant cells thereof for induced targeted loss of function mutation in the at least one gene involved in a terpene biosynthesis pathway; and
b. screening said regenerated plants for a Cannabis plant with reduced volatile organic compounds (VOCs) emission.

88. The method according to claim 85, comprising steps of:
a. introducing into a Cannabis plant or plant cells thereof a construct or expression cassette comprising (a) Cas nucleotide sequence operably linked to said at least one gRNA, or (b) a ribonucleoprotein (RNP) complex comprising Cas protein and said at least one gRNA;
b. screening the genome of said transformed plant cells for induced targeted loss of function mutation further comprises steps of obtaining a nucleic acid sample of said transformed plant and performing a nucleic acid amplification and optionally restriction enzyme digestion to detect a mutation in said at least one gene involved in a terpene biosynthesis pathway;
c. introduction into a Cannabis plant cell a construct or expression cassette comprising (a) Cas DNA and gRNA sequence selected from the group consisting of SEQ ID NO:13-646 and any combination thereof, or (b) a ribonucleoprotein (RNP) complex comprising Cas protein and gRNA sequence selected from the group consisting of SEQ ID NO:13-646 and any combination thereof; and
d. measuring or assaying the VOCs in said modified Cannabis plant using gas chromatography-mass spectrometry (GCMS) terpene profiling and quantitation techniques or by any other method for quantifying VOCs;
e. editing of CsFPPS1, CsFPPS2, CsGPPS1 and/or CsGPPS2 genes such that said editing does not involve insertion of exogenous genetic material and produces a non-naturally occurring Cannabis plant or cell thereof; and
f. reducing odor resulting from volatile organic compounds emission or generating odor free or odorless Cannabis plant.

89. The method according to claim 85, comprises silencing or eliminating Cannabis terpene synthesis gene expression comprising steps of:
a. identifying at least one gene locus within a DNA sequence in a Cannabis plant or a cell thereof for CsFPPS1, CsFPPS2, CsGPPS1 and/or CsGPPS2 having a genomic sequence as set for in SEQ ID NO:1, 4, 7 and 10, respectively;
b. identifying at least one custom endonuclease recognition sequence within the at least one locus of CsFPPS1, CsFPPS2, CsGPPS1 and/or CsGPPS2 genes;
c. introducing into the Cannabis plant or a cell thereof at least a first custom gRNA directed endonuclease, wherein the Cannabis plant or a cell thereof comprises the recognition sequence for the custom gRNA directed endonuclease in or proximal to the loci of any one of SEQ ID NO:13-646, and the custom endonuclease is expressed transiently or stably;
d. assaying the Cannabis plant or a cell thereof for a custom endonuclease-mediated modification in the DNA comprising or corresponding to or flanking the loci of any one of SEQ ID NO:13-646; and
e. identifying the Cannabis plant, a cell thereof, or a progeny cell thereof as comprising a modification in the loci of CsFPPS1, CsFPPS2, CsGPPS1 and/or CsGPPS2 genes.

90. The method according to claim 87, wherein the VOCs are at least one of:
a. selected from essential oils, secondary metabolites, terpenoids, terpenes, oxygenated and any combination thereof;
b. comprise at least one of hemiterpenes, monoterpenes, sesquiterpenes, diterpenes, sesterterpenes, triterpenes, tetraterpenes and polyterpenes; and
c. selected from pinene, alpha-pinene, beta-pinene, cis-pinane, trans-pinane, cis-pinanol, trans-pinanol, limonene; linalool; myrcene; eucalyptol; a-phellandrene; b-phellandrene; a-ocimene; b-ocimene, cis-ocimene, ocimene, delta-3-carene; fenchol; sabinene, bomeol, isobomeol, camphene, camphor, phellandrene, a-phellandrene, a-terpinene, geraniol, linalool, nerol, menthol, terpinolene, a-terpinolene, b-terpinolene, g-terpinolene, delta-terpinolene, a-terpineol, trans-2-pinanol, caryophyllene, caryophyllene oxide, humulene, a-humulene, a-bisabolene; b-bisabolene; santalol; selinene; nerolidol, bisabolol; a-cedrene, b-cedrene, b-eudesmol, eudesm-7(II)-en-4-ol, selina-3,7(II)-diene, guaiol, valencene, a-guaiene, beta-guaiene, delta-guaiene, guaiene, famesene, a-famesene, b-famesene, elemene, a-elemene, b-elemene, gamma-elemene, delta-elemene, germacrene, germacrene A, germacrene B, germacrene C, germacrene D, germacrene E, oridonin, phytol, isophytol, ursolic acid, oleanolic acid, and/or 1.5 ene compounds, including guaia-I(10),II-diene, and 1.5 ene. Guaia-1(10), 11-diene.isoprene, α-pinene, β-pinene, d-limonene, β-phellandrene, α-terpinene, α-thujene, γ-terpinene, β-myrcene, (E)-β-ocimene, (−)-limonene, (+)-α-pinene, β-caryophyllene, and α-humulene and any combination thereof.

91. A modified Cannabis plant produced by the method according to claim 85.

92. A method for reducing or eliminating odor resulting from VOCs emission from a Cannabis plant, the method comprising steps of producing a modified Cannabis plant according to claim 76.

93. A method for down regulation or silencing of Cannabis gene involved in a terpene biosynthesis pathway, which comprises utilizing the nucleotide sequence as set forth in at least one of SEQ ID NO:13-646 or a complementary sequence thereof, and any combination thereof, for introducing a targeted loss of function mutation into at least one of CsFPPS1, CsFPPS2, CsGPPS1 or CsGPPS2 gene, having genomic sequence comprising at least 80% identity to the sequence as set forth in SEQ ID NO:1, 4, 7 and 10 respectively using gene editing.

94. An isolated nucleic acid sequence having at least 75% sequence identity to a DNA sequence selected from the group consisting of SEQ ID NO:1, SEQ ID NO:2, SEQ ID NO:4, SEQ ID NO:5, SEQ ID NO:7, SEQ ID NO:8, SEQ ID NO:10, SEQ ID NO:11 and gRNA nucleic acid sequence as set forth in SEQ ID NO:13-646; or an isolated amino acid sequence having at least 75% sequence similarity to amino acid sequence selected from the group consisting of SEQ ID NO:3, SEQ ID NO:6, SEQ ID NO:9 and SEQ ID NO:12.

95. Use of a gRNA nucleotide sequence according to claim 94 for silencing at least one gene involved in terpene biosynthesis pathway, by targeted gene editing of Cannabis CsFPPS1, CsFPPS2, CsGPPS1 or CsGPPS2 encoding genes.


SEQUENCE LISTING (646 sequences)

SEQ ID NO: 1 - DNA, 4799 bp, Cannabis sativa
aatcttaaatgtgactgcccatctctgagaagactacagttttctgttccaagatttagattacacctcaccttttcaccgataatatccattttgcataattacttgtacagttgagcaaatttacgaaaataacgcaaataattggaaaacatggcatcaaaaaaattaggaaggacaataactaacccataatattagaacaacttatgtgtatgtattcttttgataaacacaaaactctgcatatatatacattcgaataaataatagaataatcttcacagatgggatccagatgaaaatgccaaacttttcttcgtggttaagtgttaactataatggtaatgtttgtaattaacgcggaaagtggacccatttacgaatgaaaatcgagtttaaatctgaaactacctcacttcgttccctctcatataagcacactcggtttacttatctctctctgtaatattttgagctctatcactcgcttccatggcggatctaaaggccaagttcttgaatgtctactccgttttgaaatcagagctccttcaagatccagctttcgaattcactgatatttctcgtcaatgggtggagcgggtactaatcttaaaacttttactttctattttttgagcttttcttagctgattagattatgtcactactgattttctttttctttcttatcataatgaggtacggatatagttttacgttataattagtagtggagtggattgagttatttaactatttcataaattggtttcaaagctttgtgatgagttagaatagttatgctttcttgttgtatgtatttgtttctatagctcttttagttgtttgtttgttgattattgtcaaaataagaaaatctaatagaagaaaaatttagatataacgcatggaaagtgctagtggaattttgattagggtgtcaaaacgtgtcatcgtgttcgtgtcatattttatgtgacccgcttttatatttcgtgtcaagtgtttcaacctattttttgactcgtgtctaattatctcaactgtaacccgacccgtaaaaaatcgtgtcgtgttcgttgctattattgaagctggaattgtaagaaaaaataataattttaagctcttatatacatttatgtatatatagtttgttatatatataataaaaattaaaaaaattaaaatatttttaattttatgctaaagaaaattaaattaatttttttttttaacttgaatcactaaatatatatttttatataattatattattattattaattttttattttattatatttaaaaaaatcgtctcatcgcattcgtgttaagcgggtcgtatcgtgttggacccgtttttatttcgtgtcaagttgtgtcgacccactttcgacccgcgtctaattttttcaaccctaactcataaaattcgtgttcgtgtggcgtgtcgaaaccattgacacccctaattttgatctttatttagtaacgttttattatatatattattacatggatgccaagtaaagtaagagcattcaatacttgtgattaaacgaataataatggtaattcctgaattcctacaaattgaaaattgtgtaaacaaaaaagaaaattgtgtaaacagagcatgactttagtttagttgcaacttgcaagcatgatcgtacagttgagtcttggctttagaattgaatgagtattagcaagaaatttaagaggctagctaaactatataattatactgcatcgaaagaatatatagttgtggactatttttttcagaaaacaagaatagatgatgcttgatgtgagcttagagggacaaaatatttatgaaaaaataaaataaaattcaaaaggctctgttattggttgtgagcttaattatcaaagtacaactcaagggtttgattcatgtatttattgttacattatggctaatttcaatgtatatgttggtttttgtgtgtaattattacatgtgagcatacagatttttatcactttgccttgtagtttcctaattgtgttgtatattttggctgcagatgacagactacaatgttcctggaggttgggattgtgcaatttttataattaaattgttgaggcaaaaataaagagttctccaaaagagggtgaaaatgactcaaaaatattgttcaaaactgcttctgatgcagctctgggatgaaaatgagagaaaattattgtttagaagtctttactgatgcatctcttgggtgatattttatgttgcagggaaattaaaccgaggcctatcagttatcgacagctacaagctgttgaaaggagaagagttgactgaagaggagatctttctagccagtgctcttggttggtgcattgaatgggtacgttaagattaattaccactctcctccttggctatcttggttggtggagcctggaattttatgaatgagagataactcgagaagcttataataactatgtcactataacgttttgcagctgcaagcatactttctggttcttgatgacattatggacaactcacacacgcggcgtggccagccttgctggtataaagttcccaaggtttgtacctcattgaataagtataatctcagttaaaaataaaataaaatcgctgttttcatgtggctaaagaaattaatatgtgaacttttatcatcaaggttggaatgattgcagcaaatgatggggttttacttagaaaccatattttcagaattcttaagaatcacttcaaggacaagccatactacgttgatctgcttgatttgttcaatgaggttattcacttacgtacattttgcctagttccaccccccccccccccccccccccttcttcttctctctctctctctcatttgcttaatcgaataaatacctcttgtataggtggagttccaaaccgcttcaggacaaatgctagacttaatttcgagtgaaggggaaaatgatctatcgaaattctcattgtcactgtgagtgaacaattgcagaagttttctgcttattaaatagtgactaaattaaggatccttttggtttttcataaaaatgagcaatccatgaaaatgttggcattatattgcagccaaatacgtaagtttggaggaaaagtgcgagcaacacatttgttttgcactttactctcgcactttactcgattataaaggaaaatagagttgtataaatagagagacatgggatgaaatgtgtttgacatatgccgttttgcattatactccataatactctgttcaatgtcccttgtaatttcaagtgtttgctaatattacatttgtttggatcacttgcagtcaccgccgcattgttcagtacaaaactgcctattattcattttacctttcagtgagtattattttatccagcaagtaaaagcatccgatgactttttttccataaacattcaatatctgtccatttcgccccttatttgcactccttgtgccaccccaaatgtacttgcatctagaatcttgttacaagatttttgggtccttcttgaaatgcctgtgaaaatcagagtcattgcttatgcatttcccatatctacacaggttgcatgtgcattgcttatgtcaggggaaaatctagaagaccatgttgatgtcaagaacattcttgttcagatgggaatctactttcaagtacaggtaagttttctgttaagcatggatagttactctcaaatttatttaattgagtatttaaagcatttatgaaacatctggtttagcaaaactttcttgtacttgaaaaaacatctatgatttacgagtgtttatgttgtgtactggttattcatcgactattatctgccttctctttctaataggacgactatttggattgctttgttgatcctaagatccttggtaaggtaagcaagctaatgttgtataattctccaccagtaaccgaaataagtctattacatttacatttttgtttaatggagtccctaatataggtcctttctcctatcagataggaacagacattgaagatttcaagtgttcttggttggttgttaaagcattggagctcagcaatgaggaacaaaagaaaatattaaatgtgagactggctctttcttaatcacattgtcacttaccttatcaaaaatcagtttgcttacttttgatgtttgttccaggagaactatggcaaggcagacccggaaaaagtagctaaagtaaaagccctctacaaggagcttgatcttgaggtgcaccatcaaactctgaaaccttcttatataaaacttaataagaacaatagatttatataccatctgcatacatatatattagtatataacccgtactttgcagggtgaatttgcagactatgagagcaagagctacgagaagctgatcacatctattgaatctcaccccagcaaagctgtgcaagcagtgttgaagtctttcttgggtaagatatacaaaaggcagaaatagatcagttatcaaattccaagaacagggaggaaggaaggaaggaaaggagcatagatcggtttgtgtctaacttttttttaccattatatcaagttgtgagtaattttttttcccctgatttggactgtcactctgtttattctgtatgcaatttgtaatttttttttaaccaaggactttgcttggtttttaaccgatgaataaataaacgacattggtaattcttactccacctcaagcatgcatgttacaaaaatattttaaagcacatcatatc

SEQ ID NO: 2 - DNA, 1023 bp, Cannabis sativa
atggcggatctaaaggccaagttcttgaatgtctactccgttttgaaatcagagctccttcaagatccagctttcgaattcactgatatttctcgtcaatgggtggagcggatgacagactacaatgttcctggagggaaattaaaccgaggcctatcagttatcgacagctacaagctgttgaaaggagaagagttgactgaagaggagatctttctagccagtgctcttggttggtgcattgaatggctgcaagcatactttctggttcttgatgacattatggacaactcacacacgcggcgtggccagccttgctggtataaagttcccaaggttggaatgattgcagcaaatgatggggttttacttagaaaccatattttcagaattcttaagaatcacttcaaggacaagccatactacgttgatctgcttgatttgttcaatgaggttgagttccaaaccgcttcaggacaaatgctagacttaatttcgagtgaaggggaaaatgatctatcgaaattctcattgtcactgcaccgccgcattgttcagtacaaaactgcctattattcattttacctttcagttgcatgtgcattgcttatgtcaggggaaaatctagaagaccatgttgatgtcaagaacattcttgttcagatgggaatctactttcaagtacaggacgactatttggattgctttgttgatcctaagatccttggtaagataggaacagacattgaagatttcaagtgttcttggttggttgttaaagcattggagctcagcaatgaggaacaaaagaaaatattaaatgagaactatggcaaggcagacccggaaaaagtagctaaagtaaaagccctctacaaggagcttgatcttgagggtgaatttgcagactatgagagcaagagctacgagaagctgatcacatctattgaatctcaccccagcaaagctgtgcaagcagtgttgaagtctttcttgggtaagatatacaaaaggcagaaatag

SEQ ID NO: 3 - protein, 340 aa, Cannabis sativa
MADLKAKFLNVYSVLKSELLQDPAFEFTDISRQWVERMTDYNVPGGKLNRGLSVIDSYKLLKGEELTEEEIFLASALGWCIEWLQAYFLVLDDIMDNSHTRRGQPCWYKVPKVGMIAANDGVLLRNHIFRILKNHFKDKPYYVDLLDLFNEVEFQTASGQMLDLISSEGENDLSKFSLSLHRRIVQYKTAYYSFYLSVACALLMSGENLEDHVDVKNILVQMGIYFQVQDDYLDCFVDPKILGKIGTDIEDFKCSWLVVKALELSNEEQKKILNENYGKADPEKVAKVKALYKELDLEGEFADYESKSYEKLITSIESHPSKAVQAVLKSFLGKIYKRQK

SEQ ID NO: 4 - DNA, 2926 bp, Cannabis sativa
gttcaaggtgctattttgtattattttagaaaatataatattcaatttatcatttaacaaaatagagtacaatttttacaaaatatttatgtatatttatataatttgtttgaaatggcaaatttgtaataaaatgataaatgaagttctaatattgaagattttaaacattatttaattggtcaatacaagtggcctccaaactccagtggtctatgaacagagtactcctacactgactctctctctctctctcttctctctctctctcttcatatatatatatacacattgtatatatagattttcagtatcacagggcaaacaagtacaaacccattcgagtcctacctttgttgtgtttgtgtttgttcttcttcttcatcaaccaaaaaaataaaaatgagtgatttaaagtccaaattcatggaggtttactacaaattgaaatctgagcttctcaatgaccctgctttcgagtttaccgatgattctcgccaatgggtcgaacaggtattcttccaatttctctaactctctccattgtttttcattttatggaatttggggttttttcctttttttttttttgttaagtctttaccagattcgtttttagtcctttttaattcatgattttgtttatgtctatttaaagatctgaacttttagcttaatgatttagtgggttgtacttagtttctttaaattagtttgattttgctaagattgagtgtcattataataagattatgctcagattttatctgatcttgttaattcaagaatctgatttttttaagtgttattttgacttttgcagatgttggattacaatgtcccaggaggtttgttttgagaatgattacaatgttttaccttgtttaaacaaatttcttattgattttgattttctttgttcttgtttggtaggtaaacttaatagagggttgtcagttattgatagttaccaaattcttaaaggaggaaaggaactaactgaagaagaaattttcttaacttctgctcttggttggtgtattgaatgggtatgcaactcatttttgggaggtaaattttttcaattcatcaattgggatttaaactcttttttaacactttgttgtttcattcaattctatgttagcttcaagcatattttcttgttcttgatgatatcatggataactcggttacacgtcgcggtcaaccttgctggtttagagtaccaaaggtgtgaccttttttgtggtttttcttatcttgtttttgtattctatgtagtgaatgattcttaacatgttttaagtgtttatgttaggttggtttgattgctgcaaatgatggcattttacttcgaaaccatattccaagaattctcaagaaacatttcaaggggaagagttactatgttgatcttcttgatttgtttaatgaggtgtgatttctttgattgattgtagtttagagcaaaatggtttaatttttgtgtgatttgtgtgacggtttgctttttcgaataggttgaattccaaacagcctcaggacaaatgattgatttgatcaccacaattgaaggagagaaagatctttcaaaatactcaattccactgtaagtgaaatatttagatctgaaattactattcaatcgaaatttcgagttggctaaaatgctgtgttgtgtgttgtgttgcagtcaccatagaattgttcagtacaagactgcttactactcattctaccttccggtatgaaaaaaaccattgcctagattgtttctaatcgagtttttgatagggctgcttgttttgatgtctcattctcttgaacactatttatgaataggttgcttgtgcattggttatggctggtgaaaatcttgataaccatgttgatgtcaagaacattcttatcgaaatgggaacctattttcaagtacaggtgagttgatggcctcatttgttactgaactttttattgtattattattgactaagaaaaccaatgacatattatattgtaggatgactatttggattgttttggccacccagatgtaattggcaaggtatgttaaaagctaaattcttttgccaagtttgattcagttgggaacttttcggttttcatttatgcccttttcgattatcagattggtacagatattgaagacttcaaatgctcttggttggttgtgaaagcacttgaaatagttaccgatgaacaaaagaagctattatttgtaagccaaatcgtttcatatgaaaatgtgtctcaatagaatgtatgttacttctgtttacatgtttgtattctgtgttttccaggagcattatggcaaagcagatgaagcatctgttaaaaaagtgaaagagttatataaaactcttgatcttgaggtgttcattcattcattcattctctttttctatccatgtttttttttttttcgagtttgtggagtaaccctttactaaattcgatttttcggttttgtttgcagggtgtgtttgcagattatgagagcacaagttaccaaaagcttaataaatcgattgaagctcacccgaaaaaagaagttcaagctgtgctcaaatctttcttggctaaaatctacaaaaggcagaagtaaagagataacaaaattgtactttgtgtgaacattagagcatttctttcactctttttaataaaagggtaacttttagtaattgtttttggttactagtcataagaattcttagtagttcattttcaatgtttcttacttaccctactaagtgtggtggatattttaatgtaatatcatgtgttttctttggatgaaaattaaataatttctaatgagaaatttgttctattattccttttctctctttttgtttttgttttcatatttacccaagttcacaaaagatttgttttt

SEQ ID NO: 5 - DNA, 1026 bp, Cannabis sativa
atgagtgatttaaagtccaaattcatggaggtttactacaaattgaaatctgagcttctcaatgaccctgctttcgagtttaccgatgattctcgccaatgggtcgaacagatgttggattacaatgtcccaggaggtaaacttaatagagggttgtcagttattgatagttaccaaattcttaaaggaggaaaggaactaactgaagaagaaattttcttaacttctgctcttggttggtgtattgaatggcttcaagcatattttcttgttcttgatgatatcatggataactcggttacacgtcgcggtcaaccttgctggtttagagtaccaaaggttggtttgattgctgcaaatgatggcattttacttcgaaaccatattccaagaattctcaagaaacatttcaaggggaagagttactatgttgatcttcttgatttgtttaatgaggttgaattccaaacagcctcaggacaaatgattgatttgatcaccacaattgaaggagagaaagatctttcaaaatactcaattccactgcaccatagaattgttcagtacaagactgcttactactcattctaccttccggcttgtgcattggttatggctggtgaaaatcttgataaccatgttgatgtcaagaacattcttatcgaaatgggaacctattttcaagtacaggatgactatttggattgttttggccacccagatgtaattggcaagattggtacagatattgaagacttcaaatgctcttggttggttgtgaaagcacttgaaatagttaccgatgaacaaaagaagctattatttgagcattatggcaaagcagatgaagcatctgttaaaaaagtgaaagagttatataaaactcttgatcttcagggtgtgtttgcagattatgagagcacaagttaccaaaagcttaataaatcgattgaagctcacccgaaaaaagaagttcaagctgtgctcaaatctttcttggctaaaatctacaaaaggcagaagtaa

SEQ ID NO: 6 - protein, 342 aa, Cannabis sativa
MSDLKSKFMEVYYKLKSELLNDPAFEFTDDSRQWVEQMLDYNVPGGKLNRGLSVIDSYQILKGGKELTEEEIFLTSALGWCIEWLQAYFLVLDDIMDNSVTRRGQPCWFRVPKVGLIAANDGILLRNHIPRILKKHFKGKSYYVDLLDLFNEVEFQTASGQMIDLITTIEGEKDLSKYSIPLHHRIVQYKTAYYSFYLPVACALVMAGENLDNHVDVKNILIEMGTYFQVQDDYLDCFGHPDVIGKIGTDIEDFKCSWLVVKALEIVTDEQKKLLFEHYGKADEASVKKVKELYKTLDLQGVFADYESTSYQKLNKSIEAHPKKEVQAVLKSFLAKIYKRQK

SEQ ID NO: 7 - DNA, 1672 bp, Cannabis sativa
ggtcccatatagtttaatataataatttttaggagtataactagctaattacaaggcgacatgtcttaagggtgttaagcaccactcgttcaatacaaaaaaaagtccaagtatacctttcttatatgcctcgtaagtaacgaccaaggcaatgaaatcccatgactaaattatagcataataaatttcaccaaaaaaaaaagccaaaaacctaacataataaataatacattaattatttcaatgtcaaacaaaaaaacggtatgtgttttttttttttttgaagcccccaaatttgttttaaaaacacatgttcgaccattgctttccttactttctttatttatataattttttttaggggagctctagagcttcatcaatggcggtttataatctatcaattaattgcagtccaagatttgttcatcatgtttacgttccacatttcacatgtaaatccaataagtcgttaagtcacgtacccatgagaataaccatgtccaaacagcatcatcattcttattttgcctccacaacagccgatgtagatgcccatctcaagcaatccatcactatcaagccaccactctcagttcacgaggccatgtacaatttcatcttttccacacctccgaatttagcaccgtcattgtgcgtggcggcgtgtgagcttgtcgggggccaccaggaccaggccatggcagcagcctccgccttgcgcgtcatccacgcagccatcttcactcatgaccacctccctttaacgggcaggcccaatccaacaagtcctgaggcagcgacccacaattcttacaacccaaatattcagctccttctcccggacgcaattgtaccttttgggttcgaattgttggccaattctgatgaccttacccataataaatcagatcggattttgcgggtcattgtagagttcacacgcacctttggatcacgaggaactattgatgctcaataccatgagaagctagccagtagatttgacgttgatagtcatgaagccaaaactgtcgggtggggccattatccctctttgaagaaggaaggtgcgatgcatgcatgcgctgctgcatgtggggccattcttggagaggcacatgaagaagaggttgagaagttgagaacttttggtctttatgtgggcatgattcaaggatatgccaatagatttataatgagcagcacagaagaaaagaaagaagcagatagaatcatcgaggagttaaccaatttggctcgccaggaactaaaatatttcgatgggagaaacttagagccattttcaacctttctttttcgtctatagaaattaagacgttttgttgtgaaattaaaaaaatgtatttaattatgttttatttgaaactagaaaactggctgcgcgttgcctacatagctagtctaattaagaatatatatataatagttaataaaataatttcattaattaatataataagtctgcgcttctcacaaaataaaaaatatattcaatagataaaattataatttttatatataaatattcttactatgtacaaaccttataatttttaagaaattttttaattaaagtagcaaaaattaaacttttggtttgcatgattatttctaaagaatcatacttttctttttctagattaacaaa

SEQ ID NO: 8 - DNA, 948 bp, Cannabis sativa
atggcggtttataatctatcaattaattgcagtccaagatttgttcatcatgtttacgttccacatttcacatgtaaatccaataagtcgttaagtcacgtacccatgagaataaccatgtccaaacagcatcatcattcttattttgcctccacaacagccgatgtagatgcccatctcaagcaatccatcactatcaagccaccactctcagttcacgaggccatgtacaatttcatcttttccacacctccgaatttagcaccgtcattgtgcgtggcggcgtgtgagcttgtcgggggccaccaggaccaggccatggcagcagcctccgccttgcgcgtcatccacgcagccatcttcactcatgaccacctccctttaacgggcaggcccaatccaacaagtcctgaggcagcgacccacaattcttacaacccaaatattcagctccttctcccggacgcaattgtaccttttgggttcgaattgttggccaattctgatgaccttacccataataaatcagatcggattttgcgggtcattgtagagttcacacgcacctttggatcacgaggaactattgatgctcaataccatgagaagctagccagtagatttgacgttgatagtcatgaagccaaaactgtcgggtggggccattatccctctttgaagaaggaaggtgcgatgcatgcatgcgctgctgcatgtggggccattcttggagaggcacatgaagaagaggttgagaagttgagaacttttggtctttatgtgggcatgattcaaggatatgccaatagatttataatgagcagcacagaagaaaagaaagaagcagatagaatcatcgaggagttaaccaatttggctcgccaggaactaaaatatttcgatgggagaaacttagagccattttcaacctttctttttcgtctatag

SEQ ID NO: 9 - protein, 315 aa, Cannabis sativa
MAVYNLSINCSPRFVHHVYVPHFTCKSNKSLSHVPMRITMSKQHHHSYFASTTADVDAHLKQSITIKPPLSVHEAMYNFIFSTPPNLAPSLCVAACELVGGHQDQAMAAASALRVIHAAIFTHDHLPLTGRPNPTSPEAATHNSYNPNIQLLLPDAIVPFGFELLANSDDLTHNKSDRILRVIVEFTRTFGSRGTIDAQYHEKLASRFDVDSHEAKTVGWGHYPSLKKEGAMHACAAACGAILGEAHEEEVEKLRTFGLYVGMIQGYANRFIMSSTEEKKEADRIIEELTNLARQELKYFDGRNLEPFSTFLFRL

SEQ ID NO: 10 - DNA, 1463 bp, Cannabis sativa
tgaggttggggactaatctgcatcaatcttaagtttttgaggacttttgccgcaattatccctaattttttatatgaacgtaaatatatatatatatttctaaataaaccaaaaataataattgatgttgtaagtttctctatatattttaatgtcagtgcatgcatgatccccttgaagaaaaaattattatagtgcatgtcacaacacaatcacacgacaagccatgtcccactttattgtatcttcttcaaattttcttcaacaaaaattatacactaagattggccatttgataaataataataaaaaaaatctatgggcctagaataatgcttgaaatttcttcaacccttcttcccttgaaatgttctaattcttttatagctaatttggtgagctcctcaactctttttagtctctcctcttctttccctctaccaattttagaatatccttgaatcatgcccacatagagaccaaacttgctcaatttttcttcttcttcttcatttgcccctccaagaatggcaccacatgtagccgcgcacgcatacaacttcccttccttttttcgacatgtgtaatcgatccaatcagcattattgttttcttcctcaccattggatctattgactaccttctcatggaattgaccatcaataatcccttgtgatccaaaggctcgcgtgatctccacaataaccttcaaaacccgatctgcatgatcatcttcgatcgggttatctaaacgggttaacaactcacacccgaaagggactattgcatctgggataagaagctcaatatttggattgtaagcgtggtgaatcattggatttggattggatctatcggttaaaggaaggtgttcgtgagtaaagctagctacatgcatgaggtgcaagctcgaggctgctgccacggcttgcctcctgtggcccccaacaagttcacacgccgccacacatagcaaaggtgctgaagttggcggcgttgtaaaggccaaattgtacatgggctcatagactgtgaaaggaggcttgacaatgatggattgcttgagatgggcctctatatcttttgttatagaggcaaaataagatgagtgatcatgatgttgaaaattttgagaggacatggtgattgtcataggcatgtgaagaaaattagatgacattgatgaggagagatggtgtggagagctagagagaaattaaggaaatatatataaggaagtaatggagtaaataggcaattattggataagaaactttaacttttccttcaatcaatttcttttcaaaaaaaatttcttttgagcattgttattgaagatcaggtgacattttcaattggttaatgatatttttttaaaaattattactttaaattatattaaattcgaaatttaattataccgataacaatataatattggaaaagtatacctttatttctta

SEQ ID NO: 11 - DNA, 864 bp, Cannabis sativa
atgtcatctaattttcttcacatgcctatgacaatcaccatgtcctctcaaaattttcaacatcatgatcactcatcttattttgcctctataacaaaagatatagaggcccatctcaagcaatccatcattgtcaagcctcctttcacagtctatgagcccatgtacaatttggcctttacaacgccgccaacttcagcacctttgctatgtgtggcggcgtgtgaacttgttgggggccacaggaggcaagccgtggcagcagcctcgagcttgcacctcatgcatgtagctagctttactcacgaacaccttcctttaaccgatagatccaatccaaatccaatgattcaccacgcttacaatccaaatattgagcttcttatcccagatgcaatagtccctttcgggtgtgagttgttaacccgtttagataacccgatcgaagatgatcatgcagatcgggttttgaaggttattgtggagatcacgcgagcctttggatcacaagggattattgatggtcaattccatgagaaggtagtcaatagatccaatggtgaggaagaaaacaataatgctgattggatcgattacacatgtcgaaaaaaggaagggaagttgtatgcgtgcgcggctacatgtggtgccattcttggaggggcaaatgaagaagaagaagaaaaattgagcaagtttggtctctatgtgggcatgattcaaggatattctaaaattggtagagggaaagaagaggagagactaaaaagagttgaggagctcaccaaattagctataaaagaattagaacatttcaagggaagaagggttgaagaaatttcaagcattattctaggcccatag

SEQ ID NO: 12 - protein, 287 aa, Cannabis sativa
MSSNFLHMPMTITMSSQNFQHHDHSSYFASITKDIEAHLKQSIIVKPPFTVYEPMYNLAFTTPPTSAPLLCVAACELVGGHRRQAVAAASSLHLMHVASFTHEHLPLTDRSNPNPMIHHAYNPNIELLIPDAIVPFGCELLTRLDNPIEDDHADRVLKVIVEITRAFGSQGIIDGQFHEKVVNRSNGEEENNNADWIDYTCRKKEGKLYACAATCGAILGGANEEEEEKLSKFGLYVGMIQGYSKIGRGKEEERLKRVEELTKLAIKELEHFKGRRVEEISSIILGP

SEQ ID NO: 13 - DNA, 20 bp, Cannabis sativa
aatagaataatcttcacaga

SEQ ID NO: 14 - DNA, 20 bp, Cannabis sativa
atagaataatcttcacagat

SEQ ID NO: 15 - DNA, 20 bp, Cannabis sativa
aaaagtttggcattttcatc

SEQ ID NO: 16 - DNA, 20 bp, Cannabis sativa
cttaaccacgaagaaaagtt

SEQ ID NO: 17 - DNA, 20 bp, Cannabis sativa
aaatgccaaacttttcttcg

SEQ ID NO: 18 - DNA, 20 bp, Cannabis sativa
tggttaagtgttaactataa

SEQ ID NO: 19 - DNA, 20 bp, Cannabis sativa
ggtaatgtttgtaattaacg

SEQ ID NO: 20 - DNA, 20 bp, Cannabis sativa
tttgtaattaacgcggaaag

SEQ ID NO: 21 - DNA, 20 bp, Cannabis sativa
ctcgattttcattcgtaaat

SEQ ID NO: 22 - DNA, 20 bp, Cannabis sativa
actcgattttcattcgtaaa

SEQ ID NO: 23 - DNA, 20 bp, Cannabis sativa
atatgagagggaacgaagtg

SEQ ID NO: 24 - DNA, 20 bp, Cannabis sativa
ccgagtgtgcttatatgaga

SEQ ID NO: 25 - DNA, 20 bp, Cannabis sativa
accgagtgtgcttatatgag

SEQ ID NO: 26 - DNA, 20 bp, Cannabis sativa
ccctctcatataagcacact

SEQ ID NO: 27 - DNA, 20 bp, Cannabis sativa
agctctatcactcgcttcca

SEQ ID NO: 28 - DNA, 20 bp, Cannabis sativa
tctatcactcgcttccatgg

SEQ ID NO: 29 - DNA, 20 bp, Cannabis sativa
cttggcctttagatccgcca

SEQ ID NO: 30 - DNA, 20 bp, Cannabis sativa
cgcttccatggcggatctaa

SEQ ID NO: 31 - DNA, 20 bp, Cannabis sativa
ggagtagacattcaagaact

SEQ ID NO: 32 - DNA, 20 bp, Cannabis sativa
aaggagctctgatttcaaaa

SEQ ID NO: 33 - DNA, 20 bp, Cannabis sativa
attcgaaagctggatcttga

SEQ ID NO: 34 - DNA, 20 bp, Cannabis sativa
atatcagtgaattcgaaagc

SEQ ID NO: 35 - DNA, 20 bp, Cannabis sativa
tcactgatatttctcgtcaa

SEQ ID NO: 36 - DNA, 20 bp, Cannabis sativa
cactgatatttctcgtcaat

SEQ ID NO: 37 - DNA, 20 bp, Cannabis sativa
tgatatttctcgtcaatggg

SEQ ID NO: 38 - DNA, 20 bp, Cannabis sativa
tttctcgtcaatgggtggag

SEQ ID NO: 39 - DNA, 20 bp, Cannabis sativa
ttctcgtcaatgggtggagc

SEQ ID NO: 40 - DNA, 20 bp, Cannabis sativa
ttttctttcttatcataatg

SEQ ID NO: 41 - DNA, 20 bp, Cannabis sativa
tttcttatcataatgaggta

SEQ ID NO: 42 - DNA, 20 bp, Cannabis sativa
ttttacgttataattagtag

SEQ ID NO: 43 - DNA, 20 bp, Cannabis sativa
cgttataattagtagtggag

SEQ ID NO: 44 - DNA, 20 bp, Cannabis sativa
ggagtggattgagttataat

SEQ ID NO: 45 - DNA, 20 bp, Cannabis sativa
aattatcaaagtacaactca

SEQ ID NO: 46 - DNA, 20 bp, Cannabis sativa
attatcaaagtacaactcaa

SEQ ID NO: 47 - DNA, 20 bp, Cannabis sativa
atgtatttattgttacatta

SEQ ID NO: 48 - DNA, 20 bp, Cannabis sativa
gctaatttcaatgtatatgt

SEQ ID NO: 49 - DNA, 20 bp, Cannabis sativa
aacacaattaggaaactaca

SEQ ID NO: 50 - DNA, 20 bp, Cannabis sativa
ccaaaatatacaacacaatt

SEQ ID NO: 51 - DNA, 20 bp, Cannabis sativa
cctaattgtgttgtatattt

SEQ ID NO: 52 - DNA, 20 bp, Cannabis sativa
atgacagactacaatgttcc

SEQ ID NO: 53 - DNA, 20 bp, Cannabis sativa
acagactacaatgttcctgg

SEQ ID NO: 54 - DNA, 20 bp, Cannabis sativa
attgcacaatcccaacctcc

SEQ ID NO: 55 - DNA, 20 bp, Cannabis sativa
ctacaatgttcctggaggtt

SEQ ID NO: 56 - DNA, 20 bp, Cannabis sativa
tttttataattaaattgttg

SEQ ID NO: 57 - DNA, 20 bp, Cannabis sativa
aataaagagttctccaaaag

SEQ ID NO: 58 - DNA, 20 bp, Cannabis sativa
ataaagagttctccaaaaga

SEQ ID NO: 59 - DNA, 20 bp, Cannabis sativa
gagtcattttcaccctcttt

SEQ ID NO: 60 - DNA, 20 bp, Cannabis sativa
aactgcttctgatgcagctc

SEQ ID NO: 61 - DNA, 20 bp, Cannabis sativa
actgcttctgatgcagctct

SEQ ID NO: 62 - DNA, 20 bp, Cannabis sativa
gtctttactgatgcatctct

SEQ ID NO: 63 - DNA, 20 bp, Cannabis sativa
tctttactgatgcatctctt

SEQ ID NO: 64 - DNA, 20 bp, Cannabis sativa
tgggtgatattttatgttgc

SEQ ID NO: 65 - DNA, 20 bp, Cannabis sativa
gggtgatattttatgttgca

SEQ ID NO: 66 - DNA, 20 bp, Cannabis sativa
gttgcagggaaattaaaccg

SEQ ID NO: 67 - DNA, 20 bp, Cannabis sativa
tgtcgataactgataggcct

SEQ ID NO: 68 - DNA, 20 bp, Cannabis sativa
tgtagctgtcgataactgat

SEQ ID NO: 69 - DNA, 20 bp, Cannabis sativa
gacagctacaagctgttgaa

SEQ ID NO: 70 - DNA, 20 bp, Cannabis sativa
aggagaagagttgactgaag

SEQ ID NO: 71 - DNA, 20 bp, Cannabis sativa
aatgcaccaaccaagagcac

SEQ ID NO: 72 - DNA, 20 bp, Cannabis sativa
atctttctagccagtgctct

SEQ ID NO: 73 - DNA, 20 bp, Cannabis sativa
ttctagccagtgctcttggt

SEQ ID NO: 74 - DNA, 20 bp, Cannabis sativa
ctcttggttggtgcattgaa

SEQ ID NO: 75 - DNA, 20 bp, Cannabis sativa
tcttggttggtgcattgaat

SEQ ID NO: 76 - DNA, 20 bp, Cannabis sativa
aagatagccaaggaggagag

SEQ ID NO: 77 - DNA, 20 bp, Cannabis sativa
ttaattaccactctcctcct

SEQ ID NO: 78 - DNA, 20 bp, Cannabis sativa
accaaccaagatagccaagg

SEQ ID NO: 79 - DNA, 20 bp, Cannabis sativa
tccaccaaccaagatagcca

SEQ ID NO: 80 - DNA, 20 bp, Cannabis sativa
actctcctccttggctatct

SEQ ID NO: 81 - DNA, 20 bp, Cannabis sativa
tcctccttggctatcttggt

SEQ ID NO: 82 - DNA, 20 bp, Cannabis sativa
tccttggctatcttggttgg

SEQ ID NO: 83 - DNA, 20 bp, Cannabis sativa
ctatcttggttggtggagcc

SEQ ID NO: 84 - DNA, 20 bp, Cannabis sativa
tctctcattcataaaattcc

SEQ ID NO: 85 - DNA, 20 bp, Cannabis sativa
gcagctgcaagcatactttc

SEQ ID NO: 86 - DNA, 20 bp, Cannabis sativa
tctggttcttgatgacatta

SEQ ID NO: 87 - DNA, 20 bp, Cannabis sativa
ttatggacaactcacacacg

SEQ ID NO: 88 - DNA, 20 bp, Cannabis sativa
gacaactcacacacgcggcg

SEQ ID NO: 89 - DNA, 20 bp, Cannabis sativa
gaactttataccagcaaggc

SEQ ID NO: 90 - DNA, 20 bp, Cannabis sativa
cgcggcgtggccagccttgc

SEQ ID NO: 91 - DNA, 20 bp, Cannabis sativa
ttgggaactttataccagca

SEQ ID NO: 92 - DNA, 20 bp, Cannabis sativa
ttgctggtataaagttccca

SEQ ID NO: 93 - DNA, 20 bp, Cannabis sativa
ttcaatgaggtacaaacctt

SEQ ID NO: 94 - DNA, 20 bp, Cannabis sativa
attcaatgaggtacaaacct

SEQ ID NO: 95 - DNA, 20 bp, Cannabis sativa
gagattatacttattcaatg

SEQ ID NO: 96 - DNA, 20 bp, Cannabis sativa
ataaaatcgctgttttcatg

SEQ ID NO: 97 - DNA, 20 bp, Cannabis sativa
tatgtgaacttttatcatca

SEQ ID NO: 98 - DNA, 20 bp, Cannabis sativa
tgaacttttatcatcaaggt

SEQ ID NO: 99 - DNA, 20 bp, Cannabis sativa
ggaatgattgcagcaaatga

SEQ ID NO: 100 - DNA, 20 bp, Cannabis sativa
gaatgattgcagcaaatgat

SEQ ID NO: 101 - DNA, 20 bp, Cannabis sativa
aatgattgcagcaaatgatg

SEQ ID NO: 102 - DNA, 20 bp, Cannabis sativa
tcttaagaattctgaaaata

SEQ ID NO: 103 - DNA, 20 bp, Cannabis sativa
aattcttaagaatcacttca

SEQ ID NO: 104 - DNA, 20 bp, Cannabis sativa
tcaagcagatcaacgtagta

SEQ ID NO: 105 - DNA, 20 bp, Cannabis sativa
tctgcttgatttgttcaatg

SEQ ID NO: 106 - DNA, 20 bp, Cannabis sativa
gggggggggggggtggaact

SEQ ID NO: 107 - DNA, 20 bp, Cannabis sativa
aagaaggggggggggggggg

SEQ ID NO: 108 - DNA, 20 bp, Cannabis sativa
gaagaagggggggggggggg

SEQ ID NO: 109 - DNA, 20 bp, Cannabis sativa
agaagaaggggggggggggg

SEQ ID NO: 110 - DNA, 20 bp, Cannabis sativa
aagaagaagggggggggggg

SEQ ID NO: 111 - DNA, 20 bp, Cannabis sativa
gaagaagaaggggggggggg

SEQ ID NO: 112 - DNA, 20 bp, Cannabis sativa
agaagaagaagggggggggg

SEQ ID NO: 113 - DNA, 20 bp, Cannabis sativa
gagaagaagaaggggggggg

SEQ ID NO: 114 - DNA, 20 bp, Cannabis sativa
agagaagaagaagggggggg

SEQ ID NO: 115 - DNA, 20 bp, Cannabis sativa
gagagaagaagaaggggggg

SEQ ID NO: 116 - DNA, 20 bp, Cannabis sativa
agagagaagaagaagggggg

SEQ ID NO: 117 - DNA, 20 bp, Cannabis sativa
gagagagaagaagaaggggg

SEQ ID NO: 118 - DNA, 20 bp, Cannabis sativa
agagagagaagaagaagggg

SEQ ID NO: 119 - DNA, 20 bp, Cannabis sativa
gagagagagaagaagaaggg

SEQ ID NO: 120 - DNA, 20 bp, Cannabis sativa
agagagagagaagaagaagg

SEQ ID NO: 121 - DNA, 20 bp, Cannabis sativa
gagagagagagaagaagaag

SEQ ID NO: 122 - DNA, 20 bp, Cannabis sativa
agagagagagagaagaagaa

SEQ ID NO: 123 - DNA, 20 bp, Cannabis sativa
gagagagagagagaagaaga

SEQ ID NO: 124 - DNA, 20 bp, Cannabis sativa
tggaactccacctatacaag

SEQ ID NO: 125 - DNA, 20 bp, Cannabis sativa
cgaataaatacctcttgtat

SEQ ID NO: 126 - DNA, 20 bp, Cannabis sativa
ataaatacctcttgtatagg

SEQ ID NO: 127 - DNA, 20 bp, Cannabis sativa
gcatttgtcctgaagcggtt

SEQ ID NO: 128 - DNA, 20 bp, Cannabis sativa
gtggagttccaaaccgcttc

SEQ ID NO: 129 - DNA, 20 bp, Cannabis sativa
gtctagcatttgtcctgaag

SEQ ID NO: 130 - DNA, 20 bp, Cannabis sativa
ctagacttaatttcgagtga

SEQ ID NO: 131 - DNA, 20 bp, Cannabis sativa
tagacttaatttcgagtgaa

SEQ ID NO: 132 - DNA, 20 bp, Cannabis sativa
agacttaatttcgagtgaag

SEQ ID NO: 133 - DNA, 20 bp, Cannabis sativa
attaaatagtgactaaatta

SEQ ID NO: 134 - DNA, 20 bp, Cannabis sativa
gactaaattaaggatccttt

SEQ ID NO: 135 - DNA, 20 bp, Cannabis sativa
catttttatgaaaaaccaaa

SEQ ID NO: 136 - DNA, 20 bp, Cannabis sativa
atataatgccaacattttca

SEQ ID NO: 137 - DNA, 20 bp, Cannabis sativa
tgagcaatccatgaaaatgt

SEQ ID NO: 138 - DNA, 20 bp, Cannabis sativa
ttcctccaaacttacgtatt

SEQ ID NO: 139 - DNA, 20 bp, Cannabis sativa
tgcagccaaatacgtaagtt

SEQ ID NO: 140 - DNA, 20 bp, Cannabis sativa
agccaaatacgtaagtttgg

SEQ ID NO: 141 - DNA, 20 bp, Cannabis sativa
cgcactttactcgattataa

SEQ ID NO: 142 - DNA, 20 bp, Cannabis sativa
gttgtataaatagagagaca

SEQ ID NO: 143 - DNA, 20 bp, Cannabis sativa
ttgtataaatagagagacat

SEQ ID NO: 144 - DNA, 20 bp, Cannabis sativa
ttatggagtataatgcaaaa

SEQ ID NO: 145 - DNA, 20 bp, Cannabis sativa
ggacattgaacagagtatta

SEQ ID NO: 146 - DNA, 20 bp, Cannabis sativa
gcaaacacttgaaattacaa

SEQ ID NO: 147 - DNA, 20 bp, Cannabis sativa
agcaaacacttgaaattaca

SEQ ID NO: 148 - DNA, 20 bp, Cannabis sativa
ttgctaatattacatttgtt

SEQ ID NO: 149 - DNA, 20 bp, Cannabis sativa
ttttgtactgaacaatgcgg

SEQ ID NO: 150 - DNA, 20 bp, Cannabis sativa
cagttttgtactgaacaatg

SEQ ID NO: 151 - DNA, 20 bp, Cannabis sativa
tgaaaggtaaaatgaataat

SEQ ID NO: 152 - DNA, 20 bp, Cannabis sativa
ataaaataatactcactgaa

SEQ ID NO: 153 - DNA, 20 bp, Cannabis sativa
catcggatgcttttacttgc

SEQ ID NO: 154 - DNA, 20 bp, Cannabis sativa
gtttatggaaaaaaagtcat

SEQ ID NO: 155 - DNA, 20 bp, Cannabis sativa
ggacagatattgaatgttta

SEQ ID NO: 156 - DNA, 20 bp, Cannabis sativa
agtgcaaataaggggcgaaa

SEQ ID NO: 157 - DNA, 20 bp, Cannabis sativa
gcacaaggagtgcaaataag

SEQ ID NO: 158 - DNA, 20 bp, Cannabis sativa
ggcacaaggagtgcaaataa

SEQ ID NO: 159 - DNA, 20 bp, Cannabis sativa
tggcacaaggagtgcaaata

SEQ ID NO: 160 - DNA, 20 bp, Cannabis sativa
agtacatttggggtggcaca

SEQ ID NO: 161 - DNA, 20 bp, Cannabis sativa
agatgcaagtacatttgggg

SEQ ID NO: 162 - DNA, 20 bp, Cannabis sativa
tctagatgcaagtacatttg

SEQ ID NO: 163 - DNA, 20 bp, Cannabis sativa
ttctagatgcaagtacattt

SEQ ID NO: 164 - DNA, 20 bp, Cannabis sativa
attctagatgcaagtacatt

SEQ ID NO: 165 - DNA, 20 bp, Cannabis sativa
gaatcttgttacaagatttt

SEQ ID NO: 166 - DNA, 20 bp, Cannabis sativa
aatcttgttacaagattttt

SEQ ID NO: 167 - DNA, 20 bp, Cannabis sativa
ttttcacaggcatttcaaga

SEQ ID NO: 168 - DNA, 20 bp, Cannabis sativa
gcaatgactctgattttcac

SEQ ID NO: 169 - DNA, 20 bp, Cannabis sativa
catgcaacctgtgtagatat

SEQ ID NO: 170 - DNA, 20 bp, Cannabis sativa
acatgcaacctgtgtagata

SEQ ID NO: 171 - DNA, 20 bp, Cannabis sativa
tgcatttcccatatctacac

SEQ ID NO: 172 - DNA, 20 bp, Cannabis sativa
gcatgtgcattgcttatgtc

SEQ ID NO: 173 - DNA, 20 bp, Cannabis sativa
catgtgcattgcttatgtca

SEQ ID NO: 174 - DNA, 20 bp, Cannabis sativa
atgtgcattgcttatgtcag

SEQ ID NO: 175 - DNA, 20 bp, Cannabis sativa
gaatgttcttgacatcaaca

SEQ ID NO: 176 - DNA, 20 bp, Cannabis sativa
caagaacattcttgttcaga

SEQ ID NO: 177 - DNA, 20 bp, Cannabis sativa
aagaacattcttgttcagat

SEQ ID NO: 178 - DNA, 20 bp, Cannabis sativa
gggaatctactttcaagtac

SEQ ID NO: 179 - DNA, 20 bp, Cannabis sativa
ggtaagttttctgttaagca

SEQ ID NO: 180 - DNA, 20 bp, Cannabis sativa
taaagcatttatgaaacatc

SEQ ID NO: 181 - DNA, 20 bp, Cannabis sativa
cgagtgtttatgttgtgtac

SEQ ID NO: 182 - DNA, 20 bp, Cannabis sativa
gtcgtcctattagaaagaga

SEQ ID NO: 183 - DNA, 20 bp, Cannabis sativa
atctgccttctctttctaat

SEQ ID NO: 184 - DNA, 20 bp, Cannabis sativa
tttctaataggacgactatt

SEQ ID NO: 185 - DNA, 20 bp, Cannabis sativa
cttaccttaccaaggatctt

SEQ ID NO: 186 - DNA, 20 bp, Cannabis sativa
tttgttgatcctaagatcct

SEQ ID NO: 187 - DNA, 20 bp, Cannabis sativa
tgatcctaagatccttggta

SEQ ID NO: 188 - DNA, 20 bp, Cannabis sativa
ttagcttgcttaccttacca

SEQ ID NO: 189 - DNA, 20 bp, Cannabis sativa
agacttatttcggttactgg

SEQ ID NO: 190 - DNA, 20 bp, Cannabis sativa
aatagacttatttcggttac

SEQ ID NO: 191 - DNA, 20 bp, Cannabis sativa
taaatgtaatagacttattt

SEQ ID NO: 192 - DNA, 20 bp, Cannabis sativa
acatttacatttttgtttaa

SEQ ID NO: 193 - DNA, 20 bp, Cannabis sativa
aggagaaaggacctatatta

SEQ ID NO: 194 - DNA, 20 bp, Cannabis sativa
taggagaaaggacctatatt

SEQ ID NO: 195 - DNA, 20 bp, Cannabis sativa
gttcctatctgataggagaa

SEQ ID NO: 196 - DNA, 20 bp, Cannabis sativa
aatgtctgttcctatctgat

SEQ ID NO: 197 - DNA, 20 bp, Cannabis sativa
ggtcctttctcctatcagat

SEQ ID NO: 198 - DNA, 20 bp, Cannabis sativa
ttgaagatttcaagtgttct

SEQ ID NO: 199 - DNA, 20 bp, Cannabis sativa
agatttcaagtgttcttggt

SEQ ID NO: 200 - DNA, 20 bp, Cannabis sativa
ttggttggttgttaaagcat

SEQ ID NO: 201 - DNA, 20 bp, Cannabis sativa
agcattggagctcagcaatg

SEQ ID NO: 202 - DNA, 20 bp, Cannabis sativa
gaaaatattaaatgtgagac

SEQ ID NO: 203 - DNA, 20 bp, Cannabis sativa
aagcaaactgatttttgata

SEQ ID NO: 204 - DNA, 20 bp, Cannabis sativa
ttacttttgatgtttgttcc

SEQ ID NO: 205 - DNA, 20 bp, Cannabis sativa
ctgccttgccatagttctcc

SEQ ID NO: 206 - DNA, 20 bp, Cannabis sativa
tgtttgttccaggagaacta

SEQ ID NO: 207 - DNA, 20 bp, Cannabis sativa
gttccaggagaactatggca

SEQ ID NO: 208 - DNA, 20 bp, Cannabis sativa
gaactatggcaaggcagacc

SEQ ID NO: 209 - DNA, 20 bp, Cannabis sativa
ttactttagctactttttcc

SEQ ID NO: 210 - DNA, 20 bp, Cannabis sativa
tttactttagctactttttc

SEQ ID NO: 211 - DNA, 20 bp, Cannabis sativa
taaagtaaaagccctctaca

SEQ ID NO: 212 - DNA, 20 bp, Cannabis sativa
caagatcaagctccttgtag

SEQ ID NO: 213 - DNA, 20 bp, Cannabis sativa
ctacaaggagcttgatcttg

SEQ ID NO: 214 - DNA, 20 bp, Cannabis sativa
aagaaggtttcagagtttga

SEQ ID NO: 215 - DNA, 20 bp, Cannabis sativa
ttattaagttttatataaga

SEQ ID NO: 216 - DNA, 20 bp, Cannabis sativa
ctaatatatatgtatgcaga

SEQ ID NO: 217 - DNA, 20 bp, Cannabis sativa
aaattcaccctgcaaagtac

SEQ ID NO: 218 - DNA, 20 bp, Cannabis sativa
caaattcaccctgcaaagta

SEQ ID NO: 219 - DNA, 20 bp, Cannabis sativa
gtatataacccgtactttgc

SEQ ID NO: 220 - DNA, 20 bp, Cannabis sativa
tatataacccgtactttgca

SEQ ID NO: 221 - DNA, 20 bp, Cannabis sativa
ctgcttgcacagctttgctg

SEQ ID NO: 222 - DNA, 20 bp, Cannabis sativa
actgcttgcacagctttgct

SEQ ID NO: 223 - DNA, 20 bp, Cannabis sativa
cactgcttgcacagctttgc

SEQ ID NO: 224 - DNA, 20 bp, Cannabis sativa
agcagtgttgaagtctttct

SEQ ID NO: 225 - DNA, 20 bp, Cannabis sativa
gcagtgttgaagtctttctt

SEQ ID NO: 226 - DNA, 20 bp, Cannabis sativa
tcttgggtaagatatacaaa

SEQ ID NO: 227 - DNA, 20 bp, Cannabis sativa
agttatcaaattccaagaac

SEQ ID NO: 228 - DNA, 20 bp, Cannabis sativa
gttatcaaattccaagaaca

SEQ ID NO: 229 - DNA, 20 bp, Cannabis sativa
atcaaattccaagaacaggg

SEQ ID NO: 230 - DNA, 20 bp, Cannabis sativa
aattccaagaacagggagga

SEQ ID NO: 231 - DNA, 20 bp, Cannabis sativa
ccaagaacagggaggaagga

SEQ ID NO: 232 - DNA, 20 bp, Cannabis sativa
gaacagggaggaaggaagga

SEQ ID NO: 233 - DNA, 20 bp, Cannabis sativa
gggaggaaggaaggaaggaa

SEQ ID NO: 234 - DNA, 20 bp, Cannabis sativa
actacaatgttcctggaggt

SEQ ID NO: 235 - DNA, 20 bp, Cannabis sativa
tttaatggagtccctaatat

SEQ ID NO: 236 - DNA, 20 bp, Cannabis sativa
aagatcaagctccttgtaga

SEQ ID NO: 237 - DNA, 20 bp, Cannabis sativa
ccttccttcctccctgttct

SEQ ID NO: 238 - DNA, 20 bp, Cannabis sativa
tttatataatttgtttgaaa

SEQ ID NO: 239 - DNA, 20 bp, Cannabis sativa
gattttaaacattatttaat

SEQ ID NO: 240 - DNA, 20 bp, Cannabis sativa
atttaattggtcaatacaag

SEQ ID NO: 241 - DNA, 20 bp, Cannabis sativa
catagaccactggagtttgg

SEQ ID NO: 242 - DNA, 20 bp, Cannabis sativa
gttcatagaccactggagtt

SEQ ID NO: 243 - DNA, 20 bp, Cannabis sativa
aagtggcctccaaactccag

SEQ ID NO: 244 - DNA, 20 bp, Cannabis sativa
gtactctgttcatagaccac

SEQ ID NO: 245 - DNA, 20 bp, Cannabis sativa
gagagagagagagtcagtgt

SEQ ID NO: 246 - DNA, 20 bp, Cannabis sativa
atatagattttcagtatcac

SEQ ID NO: 247 - DNA, 20 bp, Cannabis sativa
tatagattttcagtatcaca

SEQ ID NO: 248 - DNA, 20 bp, Cannabis sativa
aacaaaggtaggactcgaat

SEQ ID NO: 249 - DNA, 20 bp, Cannabis sativa
caacaaaggtaggactcgaa

SEQ ID NO: 250 - DNA, 20 bp, Cannabis sativa
aacacaaacacaacaaaggt

SEQ ID NO: 251 - DNA, 20 bp, Cannabis sativa
aacaaacacaaacacaacaa

SEQ ID NO: 252 - DNA, 20 bp, Cannabis sativa
atcactcatttttatttttt

SEQ ID NO: 253 - DNA, 20 bp, Cannabis sativa
gtagtaaacctccatgaatt

SEQ ID NO: 254 - DNA, 20 bp, Cannabis sativa
tttaaagtccaaattcatgg

SEQ ID NO: 255 - DNA, 20 bp, Cannabis sativa
catcggtaaactcgaaagca

SEQ ID NO: 256 - DNA, 20 bp, Cannabis sativa
tcatcggtaaactcgaaagc

SEQ ID NO: 257 - DNA, 20 bp, Cannabis sativa
gacccattggcgagaatcat

SEQ ID NO: 258 - DNA, 20 bp, Cannabis sativa
ttaccgatgattctcgccaa

SEQ ID NO: 259 - DNA, 20 bp, Cannabis sativa
taccgatgattctcgccaat

SEQ ID NO: 260 - DNA, 20 bp, Cannabis sativa
agaatacctgttcgacccat

SEQ ID NO: 261 - DNA, 20 bp, Cannabis sativa
ttctcgccaatgggtcgaac

SEQ ID NO: 262 - DNA, 20 bp, Cannabis sativa
atggagagagttagagaaat

SEQ ID NO: 263 - DNA, 20 bp, Cannabis sativa
ttccataaaatgaaaaacaa

SEQ ID NO: 264 - DNA, 20 bp, Cannabis sativa
ctccattgtttttcatttta

SEQ ID NO: 265 - DNA, 20 bp, Cannabis sativa
gtttttcattttatggaatt

SEQ ID NO: 266 - DNA, 20 bp, Cannabis sativa
tttttcattttatggaattt

SEQ ID NO: 267 - DNA, 20 bp, Cannabis sativa
ttttcattttatggaatttg

SEQ ID NO: 268 - DNA, 20 bp, Cannabis sativa
gacttaacaaaaaaaaaaaa

SEQ ID NO: 269 - DNA, 20 bp, Cannabis sativa
aaaaggactaaaaacgaatc

SEQ ID NO: 270 - DNA, 20 bp, Cannabis sativa
aacaaaatcatgaattaaaa

SEQ ID NO: 271 - DNA, 20 bp, Cannabis sativa
cttttagcttaatgatttag

SEQ ID NO: 272 - DNA, 20 bp, Cannabis sativa
ttttagcttaatgatttagt

SEQ ID NO: 273 - DNA, 20 bp, Cannabis sativa
attttgacttttgcagatgt

SEQ ID NO: 274 - DNA, 20 bp, Cannabis sativa
atgttggattacaatgtccc

SEQ ID NO: 275 - DNA, 20 bp, Cannabis sativa
ttggattacaatgtcccagg

SEQ ID NO: 276 - DNA, 20 bp, Cannabis sativa
attctcaaaacaaacctcct

SEQ ID NO: 277 - DNA, 20 bp, Cannabis sativa
cattctcaaaacaaacctcc

SEQ ID NO: 278 - DNA, 20 bp, Cannabis sativa
ataagaaatttgtttaaaca

SEQ ID NO: 279 - DNA, 20 bp, Cannabis sativa
tgattttctttgttcttgtt

SEQ ID NO: 280 - DNA, 20 bp, Cannabis sativa
tttctttgttcttgtttggt

SEQ ID NO: 281 - DNA, 20 bp, Cannabis sativa
ttggtaggtaaacttaatag

SEQ ID NO: 282 - DNA, 20 bp, Cannabis sativa
tggtaggtaaacttaataga

SEQ ID NO: 283 - DNA, 20 bp, Cannabis sativa
cctttcctcctttaagaatt

SEQ ID NO: 284 - DNA, 20 bp, Cannabis sativa
gatagttaccaaattcttaa

SEQ ID NO: 285 - DNA, 20 bp, Cannabis sativa
agttaccaaattcttaaagg

SEQ ID NO: 286 - DNA, 20 bp, Cannabis sativa
ccaaattcttaaaggaggaa

SEQ ID NO: 287 - DNA, 20 bp, Cannabis sativa
attttcttaacttctgctct

SEQ ID NO: 288 - DNA, 20 bp, Cannabis sativa
tcttaacttctgctcttggt

SEQ ID NO: 289 - DNA, 20 bp, Cannabis sativa
ctcttggttggtgtattgaa

SEQ ID NO: 290 - DNA, 20 bp, Cannabis sativa
tcttggttggtgtattgaat

SEQ ID NO: 291 - DNA, 20 bp, Cannabis sativa
atgggtatgcaactcatttt

SEQ ID NO: 292 - DNA, 20 bp, Cannabis sativa
tgggtatgcaactcattttt

SEQ ID NO: 293 - DNA, 20 bp, Cannabis sativa
gtatgcaactcatttttggg

SEQ ID NO: 294 - DNA, 20 bp, Cannabis sativa
aattttttcaattcatcaat

SEQ ID NO: 295 - DNA, 20 bp, Cannabis sativa
attttttcaattcatcaatt

SEQ ID NO: 296 - DNA, 20 bp, Cannabis sativa
tcttgttcttgatgatatca

SEQ ID NO: 297 - DNA, 20 bp, Cannabis sativa
tgatgatatcatggataact

SEQ ID NO: 298 - DNA, 20 bp, Cannabis sativa
gataactcggttacacgtcg

SEQ ID NO: 299 - DNA, 20 bp, Cannabis sativa
cacgtcgcggtcaaccttgc

SEQ ID NO: 300 - DNA, 20 bp, Cannabis sativa
tttggtactctaaaccagca

SEQ ID NO: 301 - DNA, 20 bp, Cannabis sativa
ttgctggtttagagtaccaa

SEQ ID NO: 302 - DNA, 20 bp, Cannabis sativa
cacaaaaaaggtcacacctt

SEQ ID NO: 303 - DNA, 20 bp, Cannabis sativa
caaaggtgtgaccttttttg

SEQ ID NO: 304 - DNA, 20 bp, Cannabis sativa
atgttttaagtgtttatgtt

SEQ ID NO: 305 - DNA, 20 bp, Cannabis sativa
tttaagtgtttatgttaggt

SEQ ID NO: 306 - DNA, 20 bp, Cannabis sativa
ggtttgattgctgcaaatga

SEQ ID NO: 307 - DNA, 20 bp, Cannabis sativa
tcttgagaattcttggaata

SEQ ID NO: 308 - DNA, 20 bp, Cannabis sativa
aaatgtttcttgagaattct

SEQ ID NO: 309 - DNA, 20 bp, Cannabis sativa
aattctcaagaaacatttca

SEQ ID NO: 310 - DNA, 20 bp, Cannabis sativa
attctcaagaaacatttcaa

SEQ ID NO: 311 - DNA, 20 bp, Cannabis sativa
ttctcaagaaacatttcaag

SEQ ID NO: 312 - DNA, 20 bp, Cannabis sativa
tcttcttgatttgtttaatg

SEQ ID NO: 313 - DNA, 20 bp, Cannabis sativa
gattgtagtttagagcaaaa

SEQ ID NO: 314 - DNA, 20 bp, Cannabis sativa
tttttgtgtgatttgtgtga

SEQ ID NO: 315 - DNA, 20 bp, Cannabis sativa
gacggtttgctttttcgaat

SEQ ID NO: 316 - DNA, 20 bp, Cannabis sativa
tcatttgtcctgaggctgtt

SEQ ID NO: 317 - DNA, 20 bp, Cannabis sativa
gttgaattccaaacagcctc

SEQ ID NO: 318 - DNA, 20 bp, Cannabis sativa
caaatcaatcatttgtcctg

SEQ ID NO: 319 - DNA, 20 bp, Cannabis sativa
atctttctctccttcaattg

SEQ ID NO: 320 - DNA, 20 bp, Cannabis sativa
gatttgatcaccacaattga

SEQ ID NO: 321 - DNA, 20 bp, Cannabis sativa
tctaaatatttcacttacag

SEQ ID NO: 322 - DNA, 20 bp, Cannabis sativa
attcaatcgaaatttcgagt

SEQ ID NO: 323 - DNA, 20 bp, Cannabis sativa
tcttgtactgaacaattcta

SEQ ID NO: 324 - DNA, 20 bp, Cannabis sativa
ttactactcattctaccttc

SEQ ID NO: 325 - DNA, 20 bp, Cannabis sativa
atggtttttttcataccgga

SEQ ID NO: 326 - DNA, 20 bp, Cannabis sativa
ggcaatggtttttttcatac

SEQ ID NO: 327 - DNA, 20 bp, Cannabis sativa
attagaaacaatctaggcaa

SEQ ID NO: 328 - DNA, 20 bp, Cannabis sativa
aactcgattagaaacaatct

SEQ ID NO: 329 - DNA, 20 bp, Cannabis sativa
tttctaatcgagtttttgat

SEQ ID NO: 330 - DNA, 20 bp, Cannabis sativa
ttctaatcgagtttttgata

SEQ ID NO: 331 - DNA, 20 bp, Cannabis sativa
cttgaacactatttatgaat

SEQ ID NO: 332 - DNA, 20 bp, Cannabis sativa
tgaataggttgcttgtgcat

SEQ ID NO: 333 - DNA, 20 bp, Cannabis sativa
ggttgcttgtgcattggtta

SEQ ID NO: 334 - DNA, 20 bp, Cannabis sativa
gcttgtgcattggttatggc

SEQ ID NO: 335 - DNA, 20 bp, Cannabis sativa
gaatgttcttgacatcaaca

SEQ ID NO: 336 - DNA, 20 bp, Cannabis sativa
caagaacattcttatcgaaa

SEQ ID NO: 337 - DNA, 20 bp, Cannabis sativa
aagaacattcttatcgaaat

SEQ ID NO: 338 - DNA, 20 bp, Cannabis sativa
actcacctgtacttgaaaat

SEQ ID NO: 339 - DNA, 20 bp, Cannabis sativa
gggaacctattttcaagtac

SEQ ID NO: 340 - DNA, 20 bp, Cannabis sativa
ttcaagtacaggtgagttga

SEQ ID NO: 341 - DNA, 20 bp, Cannabis sativa
aaaaagttcagtaacaaatg

SEQ ID NO: 342 - DNA, 20 bp, Cannabis sativa
cctacaatataatatgtcat

SEQ ID NO: 343 - DNA, 20 bp, Cannabis sativa
ccaatgacatattatattgt

SEQ ID NO: 344 - DNA, 20 bp, Cannabis sativa
tatattgtaggatgactatt

SEQ ID NO: 345 - DNA, 20 bp, Cannabis sativa
gatgactatttggattgttt

SEQ ID NO: 346 - DNA, 20 bp, Cannabis sativa
ccttgccaattacatctggg

SEQ ID NO: 347 - DNA, 20 bp, Cannabis sativa
ataccttgccaattacatct

SEQ ID NO: 348 - DNA, 20 bp, Cannabis sativa
cataccttgccaattacatc

SEQ ID NO: 349 - DNA, 20 bp, Cannabis sativa
tttggccacccagatgtaat

SEQ ID NO: 350 - DNA, 20 bp, Cannabis sativa
ccacccagatgtaattggca

SEQ ID NO: 351 - DNA, 20 bp, Cannabis sativa
gttcccaactgaatcaaact

SEQ ID NO: 352 - DNA, 20 bp, Cannabis sativa
tttgccaagtttgattcagt

SEQ ID NO: 353 - DNA, 20 bp, Cannabis sativa
ttgccaagtttgattcagtt

SEQ ID NO: 354 - DNA, 20 bp, Cannabis sativa
tgattcagttgggaactttt

SEQ ID NO: 355 - DNA, 20 bp, Cannabis sativa
accaatctgataatcgaaaa

SEQ ID NO: 356 - DNA, 20 bp, Cannabis sativa
taccaatctgataatcgaaa

SEQ ID NO: 357 - DNA, 20 bp, Cannabis sativa
gcccttttcgattatcagat

SEQ ID NO: 358 - DNA, 20 bp, Cannabis sativa
ttgaagacttcaaatgctct

SEQ ID NO: 359 - DNA, 20 bp, Cannabis sativa
agacttcaaatgctcttggt

SEQ ID NO: 360 - DNA, 20 bp, Cannabis sativa
taatagcttcttttgttcat

SEQ ID NO: 361 - DNA, 20 bp, Cannabis sativa
cattttcatatgaaacgatt

SEQ ID NO: 362 - DNA, 20 bp, Cannabis sativa
gtttgtattctgtgttttcc

SEQ ID NO: 363 - DNA, 20 bp, Cannabis sativa
ctgctttgccataatgctcc

SEQ ID NO: 364 - DNA, 20 bp, Cannabis sativa
tgtgttttccaggagcatta

SEQ ID NO: 365 - DNA, 20 bp, Cannabis sativa
atataaaactcttgatcttg

SEQ ID NO: 366 - DNA, 20 bp, Cannabis sativa
actcgaaaaaaaaaaaaaca

SEQ ID NO: 367 - DNA, 20 bp, Cannabis sativa
ttttttttttttcgagtttg

SEQ ID NO: 368 - DNA, 20 bp, Cannabis sativa
gaaaaatcgaatttagtaaa

SEQ ID NO: 369 - DNA, 20 bp, Cannabis sativa
cgaaaaatcgaatttagtaa

SEQ ID NO: 370 - DNA, 20 bp, Cannabis sativa
ctttactaaattcgattttt

SEQ ID NO: 371 - DNA, 20 bp, Cannabis sativa
gatttttcggttttgtttgc

SEQ ID NO: 372 - DNA, 20 bp, Cannabis sativa
atttttcggttttgtttgca

SEQ ID NO: 373 - DNA, 20 bp, Cannabis sativa
caatcgatttattaagcttt

SEQ ID NO: 374 - DNA, 20 bp, Cannabis sativa
cagcttgaacttcttttttc

SEQ ID NO: 375 - DNA, 20 bp, Cannabis sativa
acagcttgaacttctttttt

SEQ ID NO: 376 - DNA, 20 bp, Cannabis sativa
agctgtgctcaaatctttct

SEQ ID NO: 377 - DNA, 20 bp, Cannabis sativa
tcttggctaaaatctacaaa

SEQ ID NO: 378 - DNA, 20 bp, Cannabis sativa
ctttcactctttttaataaa

SEQ ID NO: 379 - DNA, 20 bp, Cannabis sativa
tttcactctttttaataaaa

SEQ ID NO: 380 - DNA, 20 bp, Cannabis sativa
taacttttagtaattgtttt

SEQ ID NO: 381 - DNA, 20 bp, Cannabis sativa
aatatccaccacacttagta

SEQ ID NO: 382 - DNA, 20 bp, Cannabis sativa
aaatatccaccacacttagt

SEQ ID NO: 383 - DNA, 20 bp, Cannabis sativa
cttacttaccctactaagtg

SEQ ID NO: 384 - DNA, 20 bp, Cannabis sativa
acttaccctactaagtgtgg

SEQ ID NO: 385 - DNA, 20 bp, Cannabis sativa
gtaatatcatgtgttttctt

SEQ ID NO: 386 - DNA, 20 bp, Cannabis sativa
caaaaacaaaaagagagaaa

SEQ ID NO: 387 - DNA, 20 bp, Cannabis sativa
aacaaatcttttgtgaactt

SEQ ID NO: 388 - DNA, 20 bp, Cannabis sativa
aaacaaatcttttgtgaact

SEQ ID NO: 389 - DNA, 20 bp, Cannabis sativa
gataagaaaaaccacaaaaa

SEQ ID NO: 390 - DNA, 20 bp, Cannabis sativa
tgatttaaagtccaaattca

SEQ ID NO: 391 - DNA, 20 bp, Cannabis sativa
attattatattaaactatat

SEQ ID NO: 392 - DNA, 20 bp, Cannabis sativa
aattattatattaaactata

SEQ ID NO: 393 - DNA, 20 bp, Cannabis sativa
agtttaatataataattttt

SEQ ID NO: 394 - DNA, 20 bp, Cannabis sativa
agtataactagctaattaca

SEQ ID NO: 395 - DNA, 20 bp, Cannabis sativa
ttacaaggcgacatgtctta

SEQ ID NO: 396 - DNA, 20 bp, Cannabis sativa
tacaaggcgacatgtcttaa

SEQ ID NO: 397 - DNA, 20 bp, Cannabis sativa
ttttttttgtattgaacgag

SEQ ID NO: 398 - DNA, 20 bp, Cannabis sativa
gcatataagaaaggtatact

SEQ ID NO: 399 - DNA, 20 bp, Cannabis sativa
acttacgaggcatataagaa

SEQ ID NO: 400 - DNA, 20 bp, Cannabis sativa
tgccttggtcgttacttacg

SEQ ID NO: 401 - DNA, 20 bp, Cannabis sativa
tgcctcgtaagtaacgacca

SEQ ID NO: 402 - DNA, 20 bp, Cannabis sativa
gtcatgggatttcattgcct

SEQ ID NO: 403 - DNA, 20 bp, Cannabis sativa
ttatgctataatttagtcat

SEQ ID NO: 404 - DNA, 20 bp, Cannabis sativa
attatgctataatttagtca

SEQ ID NO: 405 - DNA, 20 bp, Cannabis sativa
aggtttttggcttttttttt

SEQ ID NO: 406 - DNA, 20 bp, Cannabis sativa
tatttattatgttaggtttt

SEQ ID NO: 407 - DNA, 20 bp, Cannabis sativa
aatgtattatttattatgtt

SEQ ID NO: 408 - DNA, 20 bp, Cannabis sativa
ttcaatgtcaaacaaaaaaa

SEQ ID NO: 409 - DNA, 20 bp, Cannabis sativa
tgtttttaaaacaaatttgg

SEQ ID NO: 410 - DNA, 20 bp, Cannabis sativa
gtgtttttaaaacaaatttg

SEQ ID NO: 411 - DNA, 20 bp, Cannabis sativa
tgtgtttttaaaacaaattt

SEQ ID NO: 412 - DNA, 20 bp, Cannabis sativa
atgtgtttttaaaacaaatt

SEQ ID NO: 413 - DNA, 20 bp, Cannabis sativa
aaagaaagtaaggaaagcaa

SEQ ID NO: 414 - DNA, 20 bp, Cannabis sativa
ttatataaataaagaaagta

SEQ ID NO: 415 - DNA, 20 bp, Cannabis sativa
ttatttatataatttttttt

SEQ ID NO: 416 - DNA, 20 bp, Cannabis sativa
tatttatataatttttttta

SEQ ID NO: 417 - DNA, 20 bp, Cannabis sativa
atttatataattttttttag

SEQ ID NO: 418 - DNA, 20 bp, Cannabis sativa
gagctctagagcttcatcaa

SEQ ID NO: 419 - DNA, 20 bp, Cannabis sativa
ctctagagcttcatcaatgg

SEQ ID NO: 420 - DNA, 20 bp, Cannabis sativa
taaacatgatgaacaaatct

SEQ ID NO: 421 - DNA, 20 bp, Cannabis sativa
ttggatttacatgtgaaatg

SEQ ID NO: 422 - DNA, 20 bp, Cannabis sativa
tacgtgacttaacgacttat

SEQ ID NO: 423 - DNA, 20 bp, Cannabis sativa
ttggacatggttattctcat

SEQ ID NO: 424 - DNA, 20 bp, Cannabis sativa
tttggacatggttattctca

SEQ ID NO: 425 - DNA, 20 bp, Cannabis sativa
atgatgatgctgtttggaca

SEQ ID NO: 426 - DNA, 20 bp, Cannabis sativa
ataagaatgatgatgctgtt

SEQ ID NO: 427 - DNA, 20 bp, Cannabis sativa
atctacatcggctgttgtgg

SEQ ID NO: 428 - DNA, 20 bp, Cannabis sativa
ggcatctacatcggctgttg

SEQ ID NO: 429 - DNA, 20 bp, Cannabis sativa
cttgagatgggcatctacat

SEQ ID NO: 430 - DNA, 20 bp, Cannabis sativa
agtgatggattgcttgagat

SEQ ID NO: 431 - DNA, 20 bp, Cannabis sativa
tagtgatggattgcttgaga

SEQ ID NO: 432 - DNA, 20 bp, Cannabis sativa
gagtggtggcttgatagtga

SEQ ID NO: 433 - DNA, 20 bp, Cannabis sativa
gcctcgtgaactgagagtgg

SEQ ID NO: 434 - DNA, 20 bp, Cannabis sativa
atggcctcgtgaactgagag

SEQ ID NO: 435 - DNA, 20 bp, Cannabis sativa
gccaccactctcagttcacg

SEQ ID NO: 436 - DNA, 20 bp, Cannabis sativa
ggaaaagatgaaattgtaca

SEQ ID NO: 437 - DNA, 20 bp, Cannabis sativa
cggtgctaaattcggaggtg

SEQ ID NO: 438 - DNA, 20 bp, Cannabis sativa
aatgacggtgctaaattcgg

SEQ ID NO: 439 - DNA, 20 bp, Cannabis sativa
cacaatgacggtgctaaatt

SEQ ID NO: 440 - DNA, 20 bp, Cannabis sativa
cacgccgccacgcacaatga

SEQ ID NO: 441 - DNA, 20 bp, Cannabis sativa
tttagcaccgtcattgtgcg

SEQ ID NO: 442 - DNA, 20 bp, Cannabis sativa
agcaccgtcattgtgcgtgg

SEQ ID NO: 443 - DNA, 20 bp, Cannabis sativa
gtggcggcgtgtgagcttgt

SEQ ID NO: 444 - DNA, 20 bp, Cannabis sativa
tggcggcgtgtgagcttgtc

SEQ ID NO: 445 - DNA, 20 bp, Cannabis sativa
ggcggcgtgtgagcttgtcg

SEQ ID NO: 446 - DNA, 20 bp, Cannabis sativa
gcggcgtgtgagcttgtcgg

SEQ ID NO: 447 - DNA, 20 bp, Cannabis sativa
tgagcttgtcgggggccacc

SEQ ID NO: 448 - DNA, 20 bp, Cannabis sativa
ctgccatggcctggtcctgg

SEQ ID NO: 449 - DNA, 20 bp, Cannabis sativa
tgtcgggggccaccaggacc

SEQ ID NO: 450 - DNA, 20 bp, Cannabis sativa
ctgctgccatggcctggtcc

SEQ ID NO: 451 - DNA, 20 bp, Cannabis sativa
gggccaccaggaccaggcca

SEQ ID NO: 452 - DNA, 20 bp, Cannabis sativa
cggaggctgctgccatggcc

SEQ ID NO: 453 - DNA, 20 bp, Cannabis sativa
caaggcggaggctgctgcca

SEQ ID NO: 454 - DNA, 20 bp, Cannabis sativa
gtggatgacgcgcaaggcgg

SEQ ID NO: 455 - DNA, 20 bp, Cannabis sativa
tgcgtggatgacgcgcaagg

SEQ ID NO: 456 - DNA, 20 bp, Cannabis sativa
ggctgcgtggatgacgcgca

SEQ ID NO: 457 - DNA, 20 bp, Cannabis sativa
catgagtgaagatggctgcg

SEQ ID NO: 458 - DNA, 20 bp, Cannabis sativa
gaggtggtcatgagtgaaga

SEQ ID NO: 459 - DNA, 20 bp, Cannabis sativa
gcctgcccgttaaagggagg

SEQ ID NO: 460 - DNA, 20 bp, Cannabis sativa
tgggcctgcccgttaaaggg

SEQ ID NO: 461 - DNA, 20 bp, Cannabis sativa
tcatgaccacctccctttaa

SEQ ID NO: 462 - DNA, 20 bp, Cannabis sativa
gattgggcctgcccgttaaa

SEQ ID NO: 463 - DNA, 20 bp, Cannabis sativa
ggattgggcctgcccgttaa

SEQ ID NO: 464 - DNA, 20 bp, Cannabis sativa
accacctccctttaacgggc

SEQ ID NO: 465 - DNA, 20 bp, Cannabis sativa
gcctcaggacttgttggatt

SEQ ID NO: 466 - DNA, 20 bp, Cannabis sativa
tgcctcaggacttgttggat

SEQ ID NO: 467 - DNA, 20 bp, Cannabis sativa
gtcgctgcctcaggacttgt

SEQ ID NO: 468 - DNA, 20 bp, Cannabis sativa
gcccaatccaacaagtcctg

SEQ ID NO: 469 - DNA, 20 bp, Cannabis sativa
gaattgtgggtcgctgcctc

SEQ ID NO: 470 - DNA, 20 bp, Cannabis sativa
atttgggttgtaagaattgt

SEQ ID NO: 471 - DNA, 20 bp, Cannabis sativa
tatttgggttgtaagaattg

SEQ ID NO: 472 - DNA, 20 bp, Cannabis sativa
ggagaaggagctgaatattt

SEQ ID NO: 473 - DNA, 20 bp, Cannabis sativa
gggagaaggagctgaatatt

SEQ ID NO: 474 - DNA, 20 bp, Cannabis sativa
aaatattcagctccttctcc

SEQ ID NO: 475 - DNA, 20 bp, Cannabis sativa
gtacaattgcgtccgggaga

SEQ ID NO: 476 - DNA, 20 bp, Cannabis sativa
caaaaggtacaattgcgtcc

SEQ ID NO: 477 - DNA, 20 bp, Cannabis sativa
ccaaaaggtacaattgcgtc

SEQ ID NO: 478 - DNA, 20 bp, Cannabis sativa
ccggacgcaattgtaccttt

SEQ ID NO: 479 - DNA, 20 bp, Cannabis sativa
cggacgcaattgtacctttt

SEQ ID NO: 480 - DNA, 20 bp, Cannabis sativa
gccaacaattcgaacccaaa

SEQ ID NO: 481 - DNA, 20 bp, Cannabis sativa
accttttgggttcgaattgt

SEQ ID NO: 482 - DNA, 20 bp, Cannabis sativa
atgggtaaggtcatcagaat

SEQ ID NO: 483 - DNA, 20 bp, Cannabis sativa
gatctgatttattatgggta

SEQ ID NO: 484 - DNA, 20 bp, Cannabis sativa
aatccgatctgatttattat

SEQ ID NO: 485 - DNA, 20 bp, Cannabis sativa
aaatccgatctgatttatta

SEQ ID NO: 486 - DNA, 20 bp, Cannabis sativa
ttacccataataaatcagat

SEQ ID NO: 487 - DNA, 20 bp, Cannabis sativa
ataaatcagatcggattttg

SEQ ID NO: 488 - DNA, 20 bp, Cannabis sativa
taaatcagatcggattttgc

SEQ ID NO: 489 - DNA, 20 bp, Cannabis sativa
gtagagttcacacgcacctt

SEQ ID NO: 490 - DNA, 20 bp, Cannabis sativa
aatagttcctcgtgatccaa

SEQ ID NO: 491 - DNA, 20 bp, Cannabis sativa
acacgcacctttggatcacg

SEQ ID NO: 492 - DNA, 20 bp, Cannabis sativa
atctactggctagcttctca

SEQ ID NO: 493 - DNA, 20 bp, Cannabis sativa
actatcaacgtcaaatctac

SEQ ID NO: 494 - DNA, 20 bp, Cannabis sativa
atggccccacccgacagttt

SEQ ID NO: 495 - DNA, 20 bp, Cannabis sativa
agtcatgaagccaaaactgt

SEQ ID NO: 496 - DNA, 20 bp, Cannabis sativa
gtcatgaagccaaaactgtc

SEQ ID NO: 497 - DNA, 20 bp, Cannabis sativa
atgaagccaaaactgtcggg

SEQ ID NO: 498 - DNA, 20 bp, Cannabis sativa
tgaagccaaaactgtcgggt

SEQ ID NO: 499 - DNA, 20 bp, Cannabis sativa
gaagccaaaactgtcgggtg

SEQ ID NO: 500 - DNA, 20 bp, Cannabis sativa
ccttcttcaaagagggataa

SEQ ID NO: 501 - DNA, 20 bp, Cannabis sativa
gcaccttccttcttcaaaga

SEQ ID NO: 502 - DNA, 20 bp, Cannabis sativa
cgcaccttccttcttcaaag

SEQ ID NO: 503 - DNA, 20 bp, Cannabis sativa
ccattatccctctttgaaga

SEQ ID NO: 504 - DNA, 20 bp, Cannabis sativa
tatccctctttgaagaagga

SEQ ID NO: 505 - DNA, 20 bp, Cannabis sativa
catgcatgcgctgctgcatg

SEQ ID NO: 506 - DNA, 20 bp, Cannabis sativa
atgcatgcgctgctgcatgt

SEQ ID NO: 507 - DNA, 20 bp, Cannabis sativa
tgcatgcgctgctgcatgtg

SEQ ID NO: 508 - DNA, 20 bp, Cannabis sativa
gctgcatgtggggccattct

SEQ ID NO: 509 - DNA, 20 bp, Cannabis sativa
ttcatgtgcctctccaagaa

SEQ ID NO: 510 - DNA, 20 bp, Cannabis sativa
atgtggggccattcttggag

SEQ ID NO: 511 - DNA, 20 bp, Cannabis sativa
tggagaggcacatgaagaag

SEQ ID NO: 512 - DNA, 20 bp, Cannabis sativa
gttgagaagttgagaacttt

SEQ ID NO: 513 - DNA, 20 bp, Cannabis sativa
gagaacttttggtctttatg

SEQ ID NO: 514 - DNA, 20 bp, Cannabis sativa
agaacttttggtctttatgt

SEQ ID NO: 515 - DNA, 20 bp, Cannabis sativa
ctttatgtgggcatgattca

SEQ ID NO: 516 - DNA, 20 bp, Cannabis sativa
gctgctcattataaatctat

SEQ ID NO: 517 - DNA, 20 bp, Cannabis sativa
agaagcagatagaatcatcg

SEQ ID NO: 518 - DNA, 20 bp, Cannabis sativa
catcgaggagttaaccaatt

SEQ ID NO: 519 - DNA, 20 bp, Cannabis sativa
tagttcctggcgagccaaat

SEQ ID NO: 520 - DNA, 20 bp, Cannabis sativa
gttaaccaatttggctcgcc

SEQ ID NO: 521 - DNA, 20 bp, Cannabis sativa
catcgaaatattttagttcc

SEQ ID NO: 522 - DNA, 20 bp, Cannabis sativa
caggaactaaaatatttcga

SEQ ID NO: 523 - DNA, 20 bp, Cannabis sativa
aggaactaaaatatttcgat

SEQ ID NO: 524 - DNA, 20 bp, Cannabis sativa
cgaaaaagaaaggttgaaaa

SEQ ID NO: 525 - DNA, 20 bp, Cannabis sativa
tttctatagacgaaaaagaa

SEQ ID NO: 526 - DNA, 20 bp, Cannabis sativa
tttatttgaaactagaaaac

SEQ ID NO: 527 - DNA, 20 bp, Cannabis sativa
cttaattagactagctatgt

SEQ ID NO: 528 - DNA, 20 bp, Cannabis sativa
aaaatttcttaaaaattata

SEQ ID NO: 529 - DNA, 20 bp, Cannabis sativa
agtagcaaaaattaaacttt

SEQ ID NO: 530 - DNA, 20 bp, Cannabis sativa
catgaccacctccctttaac

SEQ ID NO: 531 - DNA, 20 bp, Cannabis sativa
gcatcaatcttaagtttttg

SEQ ID NO: 532 - DNA, 20 bp, Cannabis sativa
taaaaaattagggataattg

SEQ ID NO: 533 - DNA, 20 bp, Cannabis sativa
tacgttcatataaaaaatta

SEQ ID NO: 534 - DNA, 20 bp, Cannabis sativa
ttacgttcatataaaaaatt

SEQ ID NO: 535 - DNA, 20 bp, Cannabis sativa
acaacatcaattattatttt

SEQ ID NO: 536 - DNA, 20 bp, Cannabis sativa
ataataattttttcttcaag

SEQ ID NO: 537 - DNA, 20 bp, Cannabis sativa
tataataattttttcttcaa

SEQ ID NO: 538 - DNA, 20 bp, Cannabis sativa
ctataataattttttcttca

SEQ ID NO: 539 - DNA, 20 bp, Cannabis sativa
agatacaataaagtgggaca

SEQ ID NO: 540 - DNA, 20 bp, Cannabis sativa
tgaagaagatacaataaagt

SEQ ID NO: 541 - DNA, 20 bp, Cannabis sativa
ttgaagaagatacaataaag

SEQ ID NO: 542 - DNA, 20 bp, Cannabis sativa
caaaaattatacactaagat

SEQ ID NO: 543 - DNA, 20 bp, Cannabis sativa
ttttattattatttatcaaa

SEQ ID NO: 544 - DNA, 20 bp, Cannabis sativa
ataataataaaaaaaatcta

SEQ ID NO: 545 - DNA, 20 bp, Cannabis sativa
taataataaaaaaaatctat

SEQ ID NO: 546 - DNA, 20 bp, Cannabis sativa
gaaatttcaagcattattct

SEQ ID NO: 547 - DNA, 20 bp, Cannabis sativa
agaacatttcaagggaagaa

SEQ ID NO: 548 - DNA, 20 bp, Cannabis sativa
tagaacatttcaagggaaga

SEQ ID NO: 549 - DNA, 20 bp, Cannabis sativa
aaagaattagaacatttcaa

SEQ ID NO: 550 - DNA, 20 bp, Cannabis sativa
aaaagaattagaacatttca

SEQ ID NO: 551 - DNA, 20 bp, Cannabis sativa
taattcttttatagctaatt

SEQ ID NO: 552 - DNA, 20 bp, Cannabis sativa
ggagagactaaaaagagttg

SEQ ID NO: 553 - DNA, 20 bp, Cannabis sativa
aattggtagagggaaagaag

SEQ ID NO: 554 - DNA, 20 bp, Cannabis sativa
gatattctaaaattggtaga

SEQ ID NO: 555 - DNA, 20 bp, Cannabis sativa
ggatattctaaaattggtag

SEQ ID NO: 556 - DNA, 20 bp, Cannabis sativa
attcaaggatattctaaaat

SEQ ID NO: 557 - DNA, 20 bp, Cannabis sativa
ctctatgtgggcatgattca

SEQ ID NO: 558 - DNA, 20 bp, Cannabis sativa
agcaagtttggtctctatgt

SEQ ID NO: 559 - DNA, 20 bp, Cannabis sativa
gagcaagtttggtctctatg

SEQ ID NO: 560 - DNA, 20 bp, Cannabis sativa
gaagaaaaattgagcaagtt

SEQ ID NO: 561 - DNA, 20 bp, Cannabis sativa
atgtggtgccattcttggag

SEQ ID NO: 562 - DNA, 20 bp, Cannabis sativa
catgtggtgccattcttgga

SEQ ID NO: 563 - DNA, 20 bp, Cannabis sativa
acatgtggtgccattcttgg

SEQ ID NO: 564 - DNA, 20 bp, Cannabis sativa
ttcatttgcccctccaagaa

SEQ ID NO: 565 - DNA, 20 bp, Cannabis sativa
gctacatgtggtgccattct

SEQ ID NO: 566 - DNA, 20 bp, Cannabis sativa
tatgcgtgcgcggctacatg

SEQ ID NO: 567 - DNA, 20 bp, Cannabis sativa
agggaagttgtatgcgtgcg

SEQ ID NO: 568 - DNA, 20 bp, Cannabis sativa
acacatgtcgaaaaaaggaa

SEQ ID NO: 569 - DNA, 20 bp, Cannabis sativa
tacacatgtcgaaaaaagga

SEQ ID NO: 570 - DNA, 20 bp, Cannabis sativa
cgattacacatgtcgaaaaa

SEQ ID NO: 571 - DNA, 20 bp, Cannabis sativa
aagaaaacaataatgctgat

SEQ ID NO: 572 - DNA, 20 bp, Cannabis sativa
agtcaatagatccaatggtg

SEQ ID NO: 573 - DNA, 20 bp, Cannabis sativa
aaggtagtcaatagatccaa

SEQ ID NO: 574 - DNA, 20 bp, Cannabis sativa
atctattgactaccttctca

SEQ ID NO: 575 - DNA, 20 bp, Cannabis sativa
tgatggtcaattccatgaga

SEQ ID NO: 576 - DNA, 20 bp, Cannabis sativa
ggatcacaagggattattga

SEQ ID NO: 577 - DNA, 20 bp, Cannabis sativa
cgcgagcctttggatcacaa

SEQ ID NO: 578 - DNA, 20 bp, Cannabis sativa
acgcgagcctttggatcaca

SEQ ID NO: 579 - DNA, 20 bp, Cannabis sativa
aataatcccttgtgatccaa

SEQ ID NO: 580 - DNA, 20 bp, Cannabis sativa
gtggagatcacgcgagcctt

SEQ ID NO: 581 - DNA, 20 bp, Cannabis sativa
tcgggttttgaaggttattg

SEQ ID NO: 582 - DNA, 20 bp, Cannabis sativa
tcatgcagatcgggttttga

SEQ ID NO: 583 - DNA, 20 bp, Cannabis sativa
cgaagatgatcatgcagatc

SEQ ID NO: 584 - DNA, 20 bp, Cannabis sativa
tcgaagatgatcatgcagat

SEQ ID NO: 585 - DNA, 20 bp, Cannabis sativa
tctgcatgatcatcttcgat

SEQ ID NO: 586 - DNA, 20 bp, Cannabis sativa
ctgcatgatcatcttcgatc

SEQ ID NO: 587 - DNA, 20 bp, Cannabis sativa
cttcgatcgggttatctaaa

SEQ ID NO: 588 - DNA, 20 bp, Cannabis sativa
ttcgatcgggttatctaaac

SEQ ID NO: 589 - DNA, 20 bp, Cannabis sativa
gttaacaactcacacccgaa

SEQ ID NO: 590 - DNA, 20 bp, Cannabis sativa
ttaacaactcacacccgaaa

SEQ ID NO: 591 - DNA, 20 bp, Cannabis sativa
cagatgcaatagtccctttc

SEQ ID NO: 592 - DNA, 20 bp, Cannabis sativa
ccagatgcaatagtcccttt

SEQ ID NO: 593 - DNA, 20 bp, Cannabis sativa
ccgaaagggactattgcatc

SEQ ID NO: 594 - DNA, 20 bp, Cannabis sativa
cgaaagggactattgcatct

SEQ ID NO: 595 - DNA, 20 bp, Cannabis sativa
gggataagaagctcaatatt

SEQ ID NO: 596 - DNA, 20 bp, Cannabis sativa
caatatttggattgtaagcg

SEQ ID NO: 597 - DNA, 20 bp, Cannabis sativa
ttgtaagcgtggtgaatcat

SEQ ID NO: 598 - DNA, 20 bp, Cannabis sativa
gcgtggtgaatcattggatt

SEQ ID NO: 599 - DNA, 20 bp, Cannabis sativa
gtgaatcattggatttggat

SEQ ID NO: 600 - DNA, 20 bp, Cannabis sativa
tggatttggattggatctat

SEQ ID NO: 601 - DNA, 20 bp, Cannabis sativa
ggattggatctatcggttaa

SEQ ID NO: 602 - DNA, 20 bp, Cannabis sativa
tggatctatcggttaaagga

SEQ ID NO: 603 - DNA, 20 bp, Cannabis sativa
taaagctagctacatgcatg

SEQ ID NO: 604 - DNA, 20 bp, Cannabis sativa
atgcatgaggtgcaagctcg

SEQ ID NO: 605 - DNA, 20 bp, Cannabis sativa
caagctcgaggctgctgcca

SEQ ID NO: 606 - DNA, 20 bp, Cannabis sativa
gggccacaggaggcaagccg

SEQ ID NO: 607 - DNA, 20 bp, Cannabis sativa
ctgccacggcttgcctcctg

SEQ ID NO: 608 - DNA, 20 bp, Cannabis sativa
aacttgttgggggccacagg

SEQ ID NO: 609 - DNA, 20 bp, Cannabis sativa
gtgaacttgttgggggccac

SEQ ID NO: 610 - DNA, 20 bp, Cannabis sativa
gcggcgtgtgaacttgttgg

SEQ ID NO: 611 - DNA, 20 bp, Cannabis sativa
ggcggcgtgtgaacttgttg

SEQ ID NO: 612 - DNA, 20 bp, Cannabis sativa
tggcggcgtgtgaacttgtt

SEQ ID NO: 613 - DNA, 20 bp, Cannabis sativa
gtggcggcgtgtgaacttgt

SEQ ID NO: 614 - DNA, 20 bp, Cannabis sativa
agcacctttgctatgtgtgg

SEQ ID NO: 615 - DNA, 20 bp, Cannabis sativa
ttcagcacctttgctatgtg

SEQ ID NO: 616 - DNA, 20 bp, Cannabis sativa
cacgccgccacacatagcaa

SEQ ID NO: 617 - DNA, 20 bp, Cannabis sativa
catagcaaaggtgctgaagt

SEQ ID NO: 618 - DNA, 20 bp, Cannabis sativa
agcaaaggtgctgaagttgg

SEQ ID NO: 619 - DNA, 20 bp, Cannabis sativa
tgaagttggcggcgttgtaa

SEQ ID NO: 620 - DNA, 20 bp, Cannabis sativa
ctatgagcccatgtacaatt

SEQ ID NO: 621 - DNA, 20 bp, Cannabis sativa
tgtaaaggccaaattgtaca

SEQ ID NO: 622 - DNA, 20 bp, Cannabis sativa
gtaaaggccaaattgtacat

SEQ ID NO: 623 - DNA, 20 bp, Cannabis sativa
atgggctcatagactgtgaa

SEQ ID NO: 624 - DNA, 20 bp, Cannabis sativa
ggctcatagactgtgaaagg

SEQ ID NO: 625 - DNA, 20 bp, Cannabis sativa
gaaaggaggcttgacaatga

SEQ ID NO: 626 - DNA, 20 bp, Cannabis sativa
caatgatggattgcttgaga

SEQ ID NO: 627 - DNA, 20 bp, Cannabis sativa
aatgatggattgcttgagat

SEQ ID NO: 628 - DNA, 20 bp, Cannabis sativa
ctctataacaaaagatatag

SEQ ID NO: 629 - DNA, 20 bp, Cannabis sativa
ctctatatcttttgttatag

SEQ ID NO: 630 - DNA, 20 bp, Cannabis sativa
atgatgttgaaaattttgag

SEQ ID NO: 631 - DNA, 20 bp, Cannabis sativa
ttgaaaattttgagaggaca

SEQ ID NO: 632 - DNA, 20 bp, Cannabis sativa
gaggacatggtgattgtcat

SEQ ID NO: 633 - DNA, 20 bp, Cannabis sativa
aaaattagatgacattgatg

SEQ ID NO: 634 - DNA, 20 bp, Cannabis sativa
atgacattgatgaggagaga

SEQ ID NO: 635 - DNA, 20 bp, Cannabis sativa
attgatgaggagagatggtg

SEQ ID NO: 636 - DNA, 20 bp, Cannabis sativa
ggagagctagagagaaatta

SEQ ID NO: 637 - DNA, 20 bp, Cannabis sativa
aaattaaggaaatatatata

SEQ ID NO: 638 - DNA, 20 bp, Cannabis sativa
aaatatatataaggaagtaa

SEQ ID NO: 639 - DNA, 20 bp, Cannabis sativa
aaggaagtaatggagtaaat

SEQ ID NO: 640 - DNA, 20 bp, Cannabis sativa
tggagtaaataggcaattat

SEQ ID NO: 641 - DNA, 20 bp, Cannabis sativa
tttgaaaagaaattgattga

SEQ ID NO: 642 - DNA, 20 bp, Cannabis sativa
gagcattgttattgaagatc

SEQ ID NO: 643 - DNA, 20 bp, Cannabis sativa
gatcaggtgacattttcaat

SEQ ID NO: 644 - DNA, 20 bp, Cannabis sativa
ttccaatattatattgttat

SEQ ID NO: 645 - DNA, 20 bp, Cannabis sativa
taccgataacaatataatat

SEQ ID NO: 646 - DNA, 20 bp, Cannabis sativa
attgttttcttcctcaccat